(19) United States
(12) Patent Application Publication
(10) Pub. No.: US 2025/0138013 A1
(43) Pub. Date: May 1, 2025

(54) SMALL MOLECULE INHIBITOR TARGETING A LEUKEMIC STEM CELL ASSOCIATED GENE FOR HIGH-RISK AML PATIENTS

(71) Applicants: The Chinese University of Hong Kong, Hong Kong (HK); Hospital Authority, Hong Kong (HK)

(72) Inventors: Margaret Heung-Ling NG, Hong Kong (HK); Herbert PITTS, Hong Kong (HK); Kelvin Chi-Keung CHENG, Hong Kong (HK)

(21) Appl. No.: 18/735,304

(22) Filed: Jun. 6, 2024

Related U.S. Application Data
(60) Provisional application No. 63/471,422, filed on Jun. 6, 2023.

(51) Int. Cl. G01N 33/574; A61K 31/403; A61K 31/517; G01N 1/30; G01N 33/68

(52) U.S. Cl. CPC G01N 33/57426; A61K 31/403; A61K 31/517; G01N 1/30; G01N 33/57492; G01N 33/68; G01N 2474/20

(57) ABSTRACT

Disclosed a method of identifying high-risk Acute Myeloid Leukemia patients based upon the expression of a leukemic stem-cell (LSC) associated gene known as Serine Protease Inhibitor Kazal type 2 (SPINK2), the method including: (i) Immunohistochemistry (IHC)-based detection of SPINK2 protein expression, (ii) quantification of SPINK2 expression using a scoring system (range 0-16), whereby high SPINK2 is defined as a score>3 and (iii) utilization of the score to classify patients as high-risk (score>3) or low risk (score 0-3). Additionally, disclosed is a method of treating AML using a small molecule inhibitor (SMI) that selectively targets a domain of SPINK2 protein in leukemic cells highly expressing SPINK2; wherein the SMI reduces SPINK2 protein expression, alters SPINK2 target gene mRNA expression, inhibits SPINK2 function and consequently LSC proliferation/survival. A method of identifying potential candidates for SPINK2-SMI therapy to enhance treatment outcomes, whereby potential candidates refer to patients with high SPINK2 expression, is also disclosed.

### CROSS-REFERENCE TO RELATED APPLICATIONS

The present application claims the benefit of U.S. Provisional Application No. 63/471,422 filed Jun. 6, 2023, entitled “Small Molecule Inhibitor targeting a Leukemic Stem Cell Associated Gene For High-Risk AML patients”, the contents of which is incorporated herein by reference in its entirety.

### FIELD OF THE INVENTION

The present invention relates to a method for identifying high-risk Acute Myeloid Leukemia (AML) patients based upon SPINK2 protein expression quantified by immunohistochemical (IHC) scores. Patients thus identified as having higher SPINK2 expression may be benefited by a novel treatment using a small molecule inhibitor that specifically inhibits SPINK2 function in leukemic stem cells. This holds potential to improve existing treatment strategies, reduce relapse risk and premature death, and thus improve overall patient outcome.

### BACKGROUND OF THE INVENTION

Acute Myeloid Leukemia (AML) is an aggressive haematological malignancy with challenging clinical management and poor prognosis owing largely to suboptimal prognostication, therapy refractoriness and high relapse risk. However, intensive research during the past decade has contributed immensely towards enhancing the understanding of the pathological mechanisms underlying leukemogenesis and disease progression. These findings have improved prognostic assessment in patients and led to the U.S. Food and Drug Administration's (FDA) approval of novel targeted therapies into standard clinical management for specific patient subgroups. Nonetheless, the clinical outcome of a substantial proportion of patients remains poor.

Leukemic stem cells (LSC) have been identified as crucial drivers of relapse and therapy resistance, with LSC gene expression signatures predicting clinical outcomes independently. Furthermore, anti-LSC therapies hold great promise in substantially improving patient outcome since LSCs are believed to lie at the root of the disease. Therefore, clinicopathological and functional characterization of LSC-associated genes is necessary.

One of the examples of the LSC-associated gene is Serine Protease Inhibitor Kazal type 2 (SPINK2). A few studies have indeed reported SPINK2 mRNA overexpression in conjunction with poor prognosis in AML-either as a single gene or in combination with other genes. Nevertheless, in-depth analyses of its protein expression, clinicopathological associations and prognostic utility in predicting therapy responses in AML are lacking. Furthermore, and importantly, the functional role and therapeutic targetability of SPINK2 in AML remain yet to be determined.

Initial in-silico analyses of several public AML datasets have demonstrated high levels of SPINK2 mRNA expression in AML compared with normal bone marrow, particularly in functionally defined LSCs fractions. Though several members of the SPINK gene family, particularly SPINK1, have been associated with aggressive cancer phenotypes, little is known about SPINK2 in cancer and AML. Initial reports suggested SPINK2 plays an important oncogenic role in the development of lymphomas and leukemias. On the contrary, a tumor-suppressive role involving the inhibition of epithelial-mesenchymal transition is also recently described for SPINK2 in testicular cancer. In normal tissues, high SPINK2 expression has been detected in the testis and found to be crucial for normal sperm development as an acrosin inhibitor. Interestingly, the most primitive hematopoietic cells also possess markedly high levels of SPINK2, suggestive of its potential role in stemness maintenance.

Gezer et. al (2022) discussed the prognosis of Acute Myeloid Leukemia (AML) can be classified into risk groups based on their genetic changes categories and it varies widely. This situation raises the need to search for new molecular markers related to AML. Serine Protease Inhibitor Kazal type 2 (SPINK2) has recently been reported to be upregulated in AML and associated with poor outcomes by meta-analysis in a limited number of AML patients. They found that SPINK2 mRNA is upregulated in both pediatric and adult patients with AML. The receiver operating characteristic (ROC) analysis found an AUC value of 0.82 [95% confidence interval (CI): 0.685-0.946] (p=0.004) and showed that SPINK2 expression might serve as a potential biomarker for distinguishing AML from controls. However, it does not describe how the patient groups can be classified effectively in a larger cohort of patients as their research is based on a smaller cohort of patients. It is also not ventured into how SPINK2 determined in their invention could be manipulated to synthesis a molecule that specifically targets the SPINK2 to reduce premature death of the AML patients.

U.S. Pat. No. 11,111,294 B2 describes an antigen recognizing constructs against tumor associated antigens (TAA), in particular the TAA Serine protease inhibitor Kazal-type 2 (SPINK2). The T cell receptor (TCR) based molecules described in the patent are selective and specific for SPINK2-expressing cancerous diseases. This means that the constructs can differentiate between cancerous and healthy cells, reducing the risk of side effects in patients. Whilst the above method is complicated, the said method did not show how it is able to effectively classify the positive patients or able to identify high-risk patients in a large group of AML patients and the said method lacked the ability to predict the outcome of the treatment.

In addition to the above, current AML treatment outcomes are suboptimal, owing to the high relapse rates that can be attributed to the residual LSCs. The integration of LSC-targeting treatment strategies into standard first-line regimens would be necessary to eradicate LSCs and boost survival. One such recent approach is the combined venetoclax or azacitidine treatment which is currently indicated in elderly patients unfit for standard intensive chemotherapy. This regimen has demonstrated superior efficacy in patients compared to conventional treatments. Mechanistically, it specifically eradicates LSCs by targeting a unique feature of their metabolism, namely their critical reliance upon oxidative phosphorylation to sustain their energy requirements. Nonetheless, even a proportion of patients with this combination treatment eventually relapse. This is thought to be due to emergence of resistance mechanisms which develop as a result of the molecular and metabolic plasticity of LSCs. Thus, this combination only targets some LSCs while others survive and consequently drive relapse. The mechanisms for the underlying treatment refractoriness and resistance remain unclear.

Additionally, no biomarker has been established to identify patients who would most benefit from the combined venetoclax-azacitidine treatment. Thus, a new type of LSC-therapy with a predictive biomarker capacity is required for an effective treatment when being used in combination with standard chemotherapy and/or existing LSC-targeting drugs such as venetoclax-azacitidine.

In view of the poor and uncertain treatment outcome and unclear mechanisms above, there is an urgent need for identification and functional characterization of a potent prognostic biomarker and therapeutic target in AML patients. Additionally, the identification of potent prognostic markers and novel therapeutic vulnerabilities remains the key to ameliorate patient risk stratification and treatment.

### SUMMARY OF THE INVENTION

It is an objective of the present invention to provide a method for identifying and selecting high-risk AML patients in a large cohort of patients based on expression of a leukemic stem cell associated gene (LSCAG), known as SPINK2.

It is also an objective of the present invention to identify a small molecule inhibitor (SMI) that is able to selectively target SPINK2 in the selected patients as a potential treatment, thereby resulting in the eradication of leukemic stem cells and subsequently improve treatment outcomes in the identified AML patients.

Another objective of the present invention to provide a potential treatment using the SMI in the selected patients with capacity to predict outcomes of the treatment and reduce premature deaths.

Accordingly, these objectives can be achieved by following the teachings of the present invention, which relates to a method identifying high-risk Acute Myeloid Leukemia (AML) patients based upon a leukemic stem cell associated gene (LSCAG) known as Serine Protease Inhibitor Kazal type 2 (SPINK2), comprising of: obtaining specimens from the patients; performing immunohistochemistry (IHC) to detect SPINK2 expression; quantifying the SPINK2 expression using to identify the high-risk AML patients and low-risk AML patients based on scores by generating a range of IHC scores.

Additionally, these objectives can be achieved by following the teachings of the present invention, which relates to a method for inhibiting proliferation of or inducing death in a leukemic cell, or for both inhibiting proliferation and inducing death in the cell, said method comprising contacting said leukemic cell with a small molecule inhibitor (SMI) wherein said leukemic cell expresses an elevated amount of SPINK2.

These objectives can be achieved by the following teachings of the present invention, which relates to a method of treating Acute Myeloid Leukemia (AML) comprising of administering an effective amount of a pharmaceutical composition to target SPINK2 and reduce its expression in a leukemic cell.

Furthermore, these objectives also can be achieved by the following teachings of the present invention, which relates to a method for treating a patient with Acute Myeloid Leukemia (AML), the method further comprising of: administering to a patient an effective amount of the SMI to selectively target a domain of the SPINK2 in the leukemic cell which expresses SPINK2, wherein, the SMI reduces SPINK2 expression, consequently alters SPINK2 target gene mRNA expressions, hence inhibiting the cells from proliferating.

Additionally, these objectives can be achieved by the following teachings of the present invention, which relates to a pharmaceutical composition for treating Acute Myeloid Leukemia (AML) comprising of a small molecule inhibitor (SMI) or its pharmaceutically acceptable salt.

These objectives also can be achieved by the following teachings of the present invention, which relates to a small molecule inhibitor (SMI) having a chemical structure of

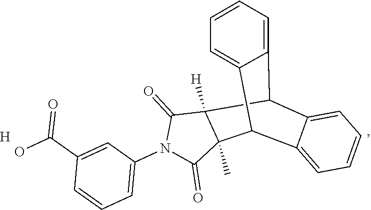

and molecular weight of 409.44 g/m and a chemical name of 3-n [(15R,19S)-15-methyl-16,18-dioxo-17-azapentacyclo [6.6.5.02,7.09,14.015,19] nonadeca-2,4,6,9,11,13-hexaen-17-yl]benzoic acid for targeting SPINK2 and reducing its expression in a leukemic cell, or for both inhibiting proliferation and inducing death in the cell.

### BRIEF DESCRIPTION OF THE DRAWINGS

The features of the invention will be more readily understood and appreciated from the following detailed description when read in conjunction with the accompanying drawings of the preferred embodiment of the present invention, in which:

FIG. 1 illustrates a 3D structure and active site of SPINK2;

FIG. 2 illustrates the SPINK2 inside a Docking box;

FIG. 3 illustrates SPINK2-ZINC100003169 small molecule interactions;

FIG. 4 illustrates SPINK2 IHC staining and expression in adult AML;

FIGS. 5A-F illustrates a prognostic refinement of ELN 2022 risk with SPINK2 IHC status;

FIGS. 6A-I illustrates a transcriptome analysis that reveals potential link between SPINK2 and ferroptosis-related genes;

FIGS. 7A-F illustrates genetic and pharmacologic modulation of SPINK2 influences sensitivity to erastin; and

FIGS. 8A-C illustrates SPINK2 knockdown modulates expression of immune-response related genes in LSC-like cells.

FIGS. 9A-F illustrates the identification of SPINK2 overexpression in AML and in LSC fractions;

FIGS. 10A-E illustrates a Univariate Kaplan-Meier (KM) survival analysis for RFS in the whole PWH cohort (N=112), the cytogenetic IR-AML (N=80), ELN 2022 IR subgroup (N=35), normal karyotype subgroup (N=62) and the NPM1mut cohort (N=35);

FIGS. 11A-D illustrates SPINK2 and outcome of SCT recipients of the transplant cohort and the TCGA-LAML;

FIGS. 12A-J illustrates a Univariate KM survival analysis for EFS and OS in the heterogeneous cohort and IR, NK-AML and NPM1mut subgroups of the PWH adult AML cohort;

FIGS. 13A-F illustrates a Univariate survival analyses (OS) of SPINK2 mRNA overexpression in the TCGA-LAML whole cohort and indicated subgroups;

FIGS. 14A-H illustrates SPINK2 and pediatric AML: Univariate survival analysis in the PWH pediatric AML cohort (N=61) and in the TARGET-AML (N=224) and Balgobind (N=193) pediatric AML datasets;

FIGS. 15A-E illustrates a modulation of SPINK2 gene expression in KG1a, ME1, GDM1, MOLM13 and OCIAML3 cells;

FIG. 16A illustrates a potential small molecule inhibitor (SMI) that binds to a target domain of SPINK2;

FIG. 16B illustrates a chemical structure of the potential SMI;

FIG. 16C illustrates the effect of the potential SMI; and,

FIGS. 17A-B compares cytotoxicity of the SMI (here designated as SMI-5) with another potential SMI identified in the screening analysis (here designated as SMI-2) in SPINK2high (KG1a, GDM1) and SPINK2low (OCIAML3, MOLM13) cell lines after 72 h of treatment which highlights the greater specificity of SMI-5 in preferentially targeting leukemic cells with higher SPINK2 expression compared with another potential SMI (SMI-2).

### DETAILED DESCRIPTION OF THE PREFERRED EMBODIMENT

For the purposes of promoting and understanding the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

Generally, the present invention relates to a method for identifying high-risk Acute Myeloid Leukemia (AML) patients based upon a leukemic stem cell associated gene (LSCAG) known as Serine Protease Inhibitor Kazal type 2 (SPINK2), comprising of: obtaining specimens from the AML patients; performing immunohistochemistry (IHC) to detect SPINK2 expression; quantifying the SPINK2 expression to identify the high-risk AML patients and low-risk AML patients by generating a range of IHC scores.

More specifically, in one embodiment, the step of quantifying the SPINK2 expression to identify the high-risk AML patients and low-risk AML patients by generating the range of the IHC scores further comprising of generating the scores ranging from minimum 0 to maximum 16 based on level of the SPINK2 expression; and, classifying the patients based on the level of the SPINK2 expression as “high-risk” if the patients score more than 3, or “low-risk” if the patients score less than or equal to 3.

In another embodiment, the SPINK2 expression also serves as a biomarker configured to determine level of risks of the AML patients.

In another embodiment, the step of performing immunohistochemistry (IHC) to detect SPINK2 expression further comprising of: preparing stained slides with collected specimens and SPINK2 antibody including visualising using an IHC Detection Kit; assessing the SPINK2-stained slides by employing percentage of stained blasts (P) with values of P: <20%=1, 20-50%=2, 50-75%=3, >75%=4 and intensity of staining (I) with values of I: negative-0, mild-1, moderate-2, strong-3, very strong-4; and, calculating a unique IHC-score as “P×1” for each patient to obtain the IHC score.

Another embodiment of the present invention is that it relates to a method for inhibiting proliferation of and inducing death in a leukemic cell comprising of: contacting said leukemic cell with a small molecule inhibitor (SMI) wherein said leukemic cell expresses an elevated amount of SPINK2.

In another embodiment, the SMI is screened via a structure-based virtual screening (SBVS) and selected from a group of bioactive molecules due to its efficient binding affinity based on its idock scores and its capacity to dissolve in at least one solvent selected from a group comprising of dimethyl sulfoxide (DMSO), water, ethanol or dimethylformamide (DMF).

Another embodiment of the present invention is that it relates to a method for treating a patient with high-risk Acute Myeloid Leukemia (AML) identified as potential candidate for receiving a small molecule (SMI) therapy based upon SPINK2 IHC score, the method further comprising of: administering to a patient an effective amount of the SMI, wherein, the effective amount of the SMI selectively targets a domain of the SPINK2 in the leukemic cell which expresses SPINK2, and, the SMI reduces SPINK2 expression, consequently alters SPINK2 target gene mRNA expressions, thus inhibiting proliferation of and inducing death in the leukemic cell.

In another embodiment, wherein the SMI is administered to the patient as a single agent or in a combination with an existing treatment regimen including but not limited to erastin.

In another embodiment, the altered SPINK2 target gene mRNA expressions are downregulation of SLC7All and upregulation of STEAP3.

Alternatively, another embodiment of the present invention also relates to a pharmaceutical composition for treating Acute Myeloid Leukemia (AML) comprising of an effective amount of a small molecule inhibitor (SMI), or its pharmaceutically acceptable salt. In this case, Dimethyl sulfoxide (DMSO), Nutlin-3a and Puromycin are added in the composition to study the functions of SPINK2 in AML cells and the effects of SMI treatment on these cells.

In another embodiment of the present invention, the composition is further comprising of an existing treatment regimen including but not limited to erastin,

Another embodiment of the present invention is that it relates to a small molecule inhibitor (SMI) having a chemical structure of

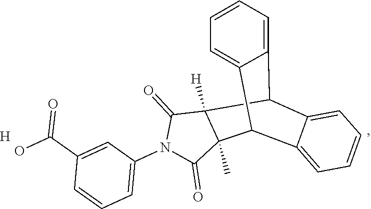

and molecular weight of 409.44 g/m and a chemical name of 3-[(15R,19S)-15-methyl-16,18-dioxo-17-azapentacyclo [6.6.5.02,7.09, 14.015,19] nonadeca-2,4,6,9,11,13-hexaen-17-yl]benzoic acid for targeting SPINK2 and reducing its expression in a leukemic cell, or for both inhibiting proliferation and inducing death in the cell.

Further aspects of the present invention are as below.

SPINK2 over-expression has shown its potential as an independent biomarker in predicting poor prognosis across wide risk groups and therefore it is selected as therapeutic target by the novel SMI for the treatment of AML.

The present invention provides several advantages over existing prognostic classification schemes and treatments. For example, the present invention provides a wide range of application across various genetic subgroups. The biomarker (SPINK2) also enables the identification of high-risk blood cancer patients (defined as SPINK2 IHC score>3) who might benefit from this novel SMI treatment. The said SMI has a great potential to be developed as effective targeting therapy to turn around treatment outcome.

In another embodiment, the screening for the novel LSC-associated oncogene further comprising analysing a plurality of databases for a gene having elevated expression in AML, and especially in functionally defined LSCs.

In another embodiment, the identified potent prognostic marker is Serine Protease Inhibitor Kazal type 2 (SPINK2).More specifically, the IHC scoring is performed for SPINK2 expression by sectioning and staining of specimens on positively charged glass slides; deparaffinizing, rehydrating and retrieving antigen using a CC1 antigen retrieval solution; incubating rabbit polyclonal primary SPINK2 antibody HPA026813 at a dilution of 1:100; visualizing using a IHC Detection Kit; incubating with hydrogen peroxide and diaminobenzidine (DAB) and copper enhancement thereafter; counterstaining with haematoxylin followed by bluing agent and manual dehydration; and covering the slides and warming prior to microscopic analysis wherein normal testicular tissue served as a positive control (with buffer and primary antibody) and negative control (with buffer, without primary antibody). The assessment of the SPINK2-stained slides is done by a qualified hematopathologist.

IHC-scoring system provides several advantages. For example, the present invention determines the classification of AML patients based on the IHC score, instead of qPCR. The IHC score is calculated by measuring the protein expression at the cellular level, therefore, the results will be more accurate. The present invention also allows the possibility of using archival samples. Utilizing such measurement would allow more accessibility to the more sophisticated classification in the present invention and significantly lower the measurement costs.

In addition to the above, the present invention provides a method of identifying potential candidates for SPINK2-SMI therapy to enhance treatment outcomes, whereby potential candidates refer to patients with high SPINK2 expression (IHC score>3).

To date, no other SMIs targeting SPINK2 expression have been taught. Furthermore, this particular SMI has also not been taught. Targeting specific LSC associated genes is desirable to increase therapy response and prevent relapse. The present invention teaches a small-molecule inhibitor (SMI) for specific targeting of a high-risk marker, SPINK2, in AML. SPINK2 in the present invention could be utilized to target leukemic stem cell associated gene (LSCAG) in cancer treatment.

It is summarized that the present invention provides strong clinical evidence of SPINK2 protein expression as a potent biomarker in AML. SPINK2 expression could refine prognostic stratification according to ELN 2022 criteria and is an indicator of elevated relapse risk and therapy resistance. Functionally, SPINK2 is potentially involved in protecting leukemic cells from cell death by ferroptosis and enhancing their immune-evasive ability.

Further elaborations of the present invention are illustrated based on the subsequent experimental designs:

### 1.1.Selection of Novel LSC-Associated Oncogenes

First, a potential novel LSC-associated oncogene is screened and analysed virtually using idock program and the number of ligands screened was 1510000 through several AML datasets from the Oncomine and NCBI GEO databases and SPINK2 is selected. A SPINK2 Virtual Screening Report for is generated. Based on the report, SPINK2 has the following protein sequence:

PQFGLFSKYRTPNCSQYRLPGCPRHFNPVCGSDMSTYANECTLCMKIREGGHNIKIIR NGPC

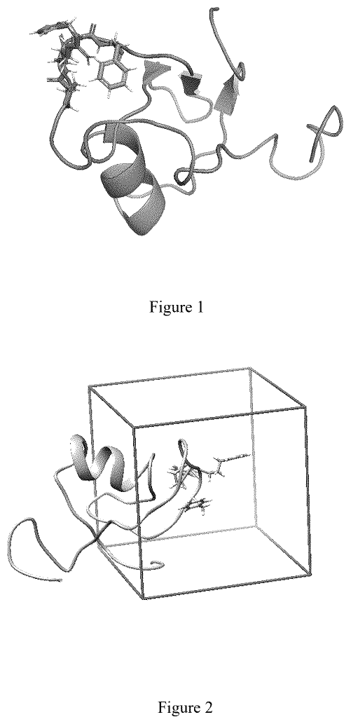

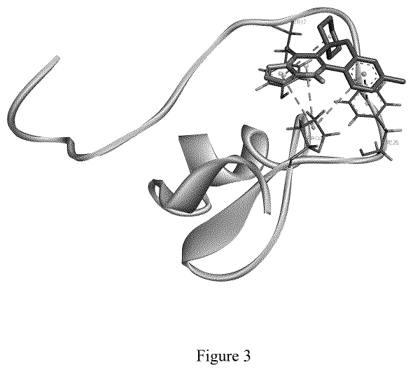

The SPINK2 protein has been reported in a 3D structure (PDB ID: 2JXD) and the same is depicted in FIG. 1, along with its the active site-SPINK2 protein inhibiting protease, specifically the PR (24) HF. Then the docking grid box center (as shown in FIG. 2) coordinates to 2.05, 4.25,-12.3 and the docking grid box side lengths to 25.0. A total of 150,000 ligands are screened and the results are saved as sorted.all.log.csv in ascending order according to idock score; the top 1000 results are taken and saved in the top 1000 folder, and the complex models of these 1000 results are generated and saved in the top 1000models. The top 10 small molecules (saved in the top 10 folder) are analyzed for their interactions (see FIG. 3).

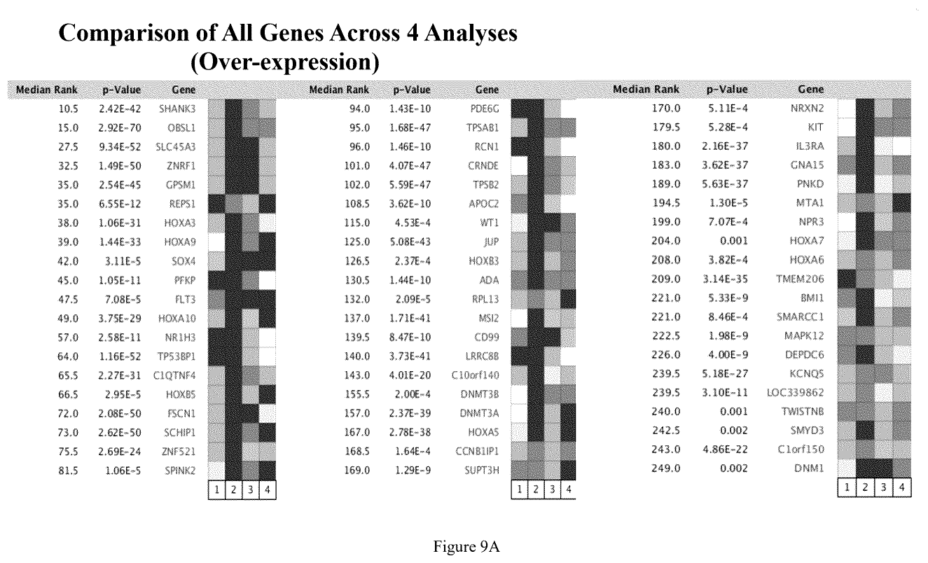

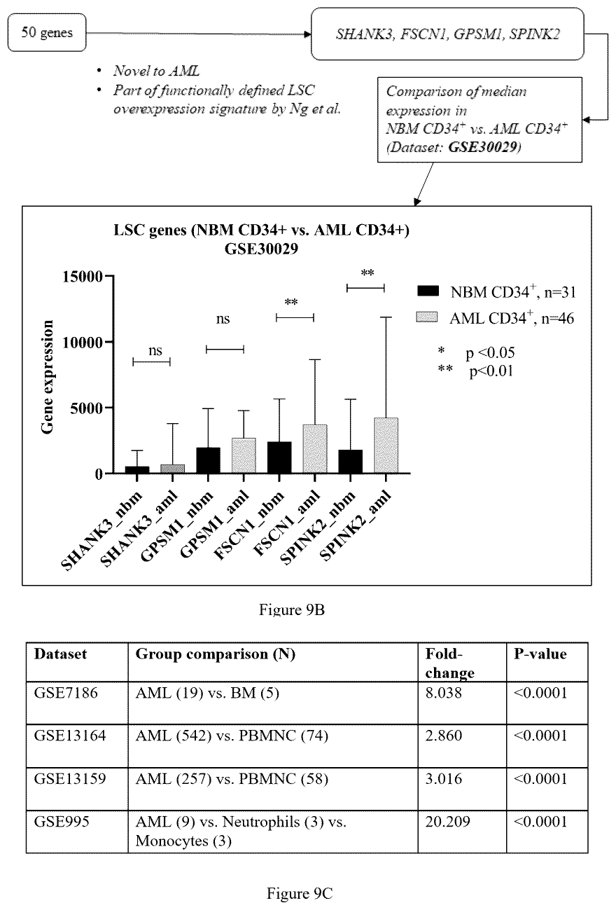

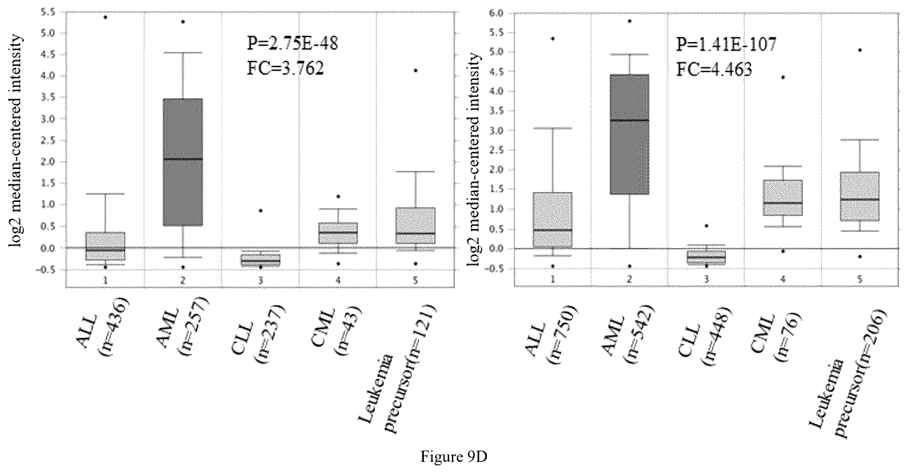

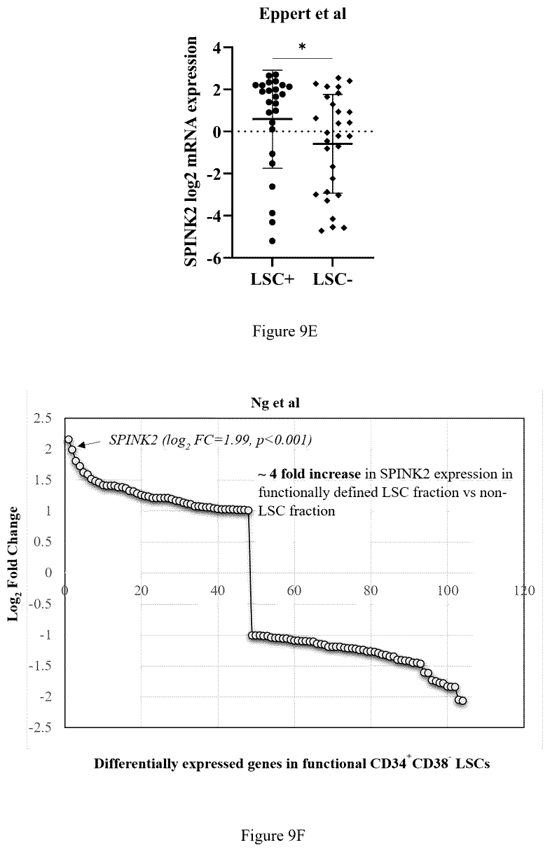

In view of the above, SPINK2 is selected due to its elevated expression in AML compared to other leukemias and, particularly, its high expression in functionally defined LSC fractions (FIGS. 9A-F). This suggested specific and important roles for SPINK2 in AML pathophysiology. However, the significance of SPINK2 in AML and other cancers remains incompletely understood.

SPINK2 protein expression is studied in a large cohort of adult AML patients by immunohistochemistry (IHC) and its clinicopathological and biological significance in AML is investigated. These analyses revealed that SPINK2 protein expression by IHC is an independent prognostic marker and could refine current ELN 2022 prognostic stratification. Furthermore, the potential functional roles of SPINK2 are identified, such as regulating ferroptosis, a non-apoptotic iron-mediated form of programmed cell death, hence suggesting new therapeutic opportunities for this aggressive hematological malignancy.

The present invention shows the results of a detailed clinicopathological investigation and functional assessment of an LSC-associated gene, SPINK2, in AML.

Generally, high SPINK2 expression is detected in intermediate-risk, normal karyotype and NPM1^{mut} subgroups. Among these subgroups, SPINK2 expression could identify high-risk patients. Notably, these genetic categories constitute large proportions of AML patients with high clinical heterogeneity, in need of potent biomarkers to refine prognostication and guide therapy decisions. The prognostic effect of SPINK2 in the whole cohort is independent of potent markers such as age, cytogenetics, ELN 2022 adverse risk, and complete remission at 1st induction. SPINK2 status could also refine risk stratification by ELN 2022 criteria which identifies higher risk patients among those classified as favorable or intermediate. Additionally, no significant correlation is detected between SPINK2 expression and known high-risk mutations such as RUNX1, ASXL1 and TP53 mutations. Thus, SPINK2 protein expression might indeed provide important added prognostic value in AML. SPINK2 is also linked to therapy resistance and increased relapse rates in adult AML patients. High SPINK2 expression associates with resistance to standard induction using daunorubicin and cytarabine, and is an independent marker for relapse. Patients with SPINK2^{high} status are at higher risk of early relapse after achieving CR. High SPINK2 status additionally predicted worse OS in SCT recipients, particularly in those receiving SCT in relapse after CR1 or in a primary refractory status. Given high SPINK2 expression is found in functionally-defined LSCs, the findings further implicate SPINK2 in AML pathophysiology, revealing its involvement in cytoprotective mechanisms allowing persistence of LSCs after therapy, thereby leading to relapse and aggressive disease.

The preliminary functional assessment in AML cell lines revealed novel potential functional roles of SPINK2, namely in regulation of ferroptosis and immune response. Ferroptosis is a morphologically distinct form of programmed cell death that involves the iron-dependent lipid peroxidation of cell membranes. Since its discovery a decade ago, ferroptosis has attracted great attention in the scientific community, and numerous studies have demonstrated its involvement in various pathophysiological (cancer, infection, autoimmune diseases) and physiological processes. Ferroptosis induction represents a novel and promising therapeutic vulnerability in cancer, as well as in eliminating cancer stem cells.

One of the primary cellular anti-ferroptotic defense mechanisms involves the SLC7A11-GPX4-GSH. SLC7A11 associates with SLC3A2 to form the xCT complex which imports cystine into the cells, and is considered the major source of intracellular cysteine and glutathione. SLC7A11 likely plays an important role in LSC biology, since its overexpression has been linked to poor prognosis in AML and LSCs are critically reliant on cysteine for sustenance of their energy metabolism. Anti-ferroptotic defense mechanisms thus represent a crucial survival strategy in AML cells, since ferroptosis induction has been found to increase their sensitivity to chemotherapy. The transcriptomic analysis uncovered a link between SPINK2 and SLC7A11. Modulation of SPINK2 expression affected SLC7A11 expression and resulted in functional consequences attributable to SLC7A11, such as cystine uptake and altered sensitivity to erastin, a ferroptosis inducer. The data also suggests that SPINK2 is involved in suppression of p53-mediated ferroptosis induction. The tumor-suppressor, p53, is now a well-known master regulator of ferroptosis and transcriptional repressor of SLC7A11. The expression of another p53 target, STEAP3, which is pro-ferroptotic and increases intracellular Fe²⁺, is also affected by SPINK2 modulation with resultant functional changes (i.e., increased Fe²⁺ levels).

Evading the immune system is a hallmark of cancer and an important survival mechanism employed by AML blasts and LSCs. Of note, analysis of in-silico data by a recent study discovered a link between SPINK2 and immune regulation via PI3K-AKT signalling and PD-L1 expression. The present invention provides functional evidence showing that SPINK2 regulates expression of immune-response related genes, particularly in LSC-like cells.

SPINK2 knockdown consistently increased expression of ALCAM in three LSC-like cell lines, namely, KG1a, ME1 and GDM1. ALCAM, an immunoglobulin superfamily protein, is expressed by antigen presenting cells (APCs) and is a specific ligand of the CD6 receptor on CD4+ T-cells. The CD6/ALCAM interaction is crucial for establishment of the immunological synapse, which promotes T-cell activation and proliferation. GSEA analysis of the RNA-seq data further showed that several pathways associated with regulation of the immune response are affected by SPINK2 knockdown and overexpression. SPINK2 thus serves to mitigate the immune response by modulating expression of genes associated with T-cell activity, especially ALCAM expression. SPINK2 is normally highly expressed in the testis, where it is essential for normal spermiogenesis and where the spermatozoa must be protected from eradication by the immune system. It is thus reasonable to infer that high SPINK2 expression in LSCs might help boost their survival against the host immune system.

Interestingly, recent studies have also demonstrated a link between anti-tumor immune response and ferroptosis. For example, activated CD8+ T-cells induced ferroptotic cell death in cancer cells by downregulating SLC7A11 expression through interferon-gamma secretion. Given the between SPINK2, ferroptosis and immune response, it is in need to further grasp and utilize the functions of SPINK2 in this context in an in vivo model.

Finally, a potential SPINK2 small molecule inhibitor (SMI) which selectively decreased viability of high SPINK2 expressing cells (KG1a, GDM1), decreased SPINK2 protein expression, altered expression of SPINK2 targets (SLC7A11 and STEAP3) and increased erastin sensitivity are identified by the present invention. Further functional characterization of this SMI is determined contributing to its therapeutic potential and described below are the materials and methods, along with the results obtained throughout the present invention.

### 1.2.Assessment of SPINK2 Protein Expression

SPINK2 expression and its clinicopathological associations in AML are determined using IHC and next-generation sequencing (NGS) in the cohort comprising of 172 AML patients treated at the Prince of Wales Hospital (PWH). IHC for SPINK2 is performed on diagnostic BM specimens of non-M3 patients (median age: 52yrs, range: 18-86yrs). The majority are de novo AML (90.8%), with 72.3% having intermediate-risk (IR) cytogenetics according to the Medical Research Council (MRC) classification. Table S1 summarizes their baseline characteristics. DNA is available for 152 patients, and is sequenced by NGS using a targeted myeloid panel covering 141 commonly mutated genes in myeloid neoplasms. Based upon data availability, public datasets (TCGA-LAML, OHSU-Beat AML, TARGET-AML) are also analysed for clinicopathological and prognostic correlations. Details of these datasets, and exclusion criteria for survival and treatment-response analyses are found in Supplementary information.

TABLE S1
Supplementary Table S1. Baseline characteristics
of the 172 adult AML patients of the PWH cohort

| | |
|---|---|
| Characteristic | Value |

Age

| | | |
|---|---|---|
| Range (median), yrs. | 18-86 | (52) |
| Gender: Male (%) | 89 | (52%) |
| Female (%) | 83 | (48%) |

BM Blast %,

| | | |
|---|---|---|
| Range (median) | 11-98 | (69) |

WBC count, ×10⁹/L

| | | |
|---|---|---|
| Range (median) | 0.9-517 | (24.5) |

AML type

| | | |
|---|---|---|
| De novo AML (%) | 156 | (90.8%) |
| secondary/therapy-related | 16 | (9.2%) |
| AML (%) | | |

FAB subtype

| | | |
|---|---|---|
| M0 | 5 | (2.9%) |
| M1 | 32 | (18.6%) |
| M2 | 25 | (14.5%) |
| M4 (incl. M4Eo) | 24 | (14.0%) |
| M5 | 27 | (15.7%) |
| M6 | 3 | (1.7%) |
| Unclassified | 56 | (32.6%) |

Cytogenetics subgroups

| | | |
|---|---|---|
| Normal (%) | 96 | (55.8%) |
| Non-Normal (%) | 73 | (42.4%) |
| Unknown (%) | 3 | (1.7%) |

MRC Cytogenetic Risk Category

| | | |
|---|---|---|
| Favorable (%) | 24 | (14.0%) |
| Intermediate (%) | 125 | (72.7%) |
| Adverse (%) | 20 | (11.6%) |
| Unknown (%) | 3 | (1.7%) |

Primary induction therapy

| | | |
|---|---|---|
| Standard (Daunorubicin + | 152 | (88.4%) |
| cytarabine, ‘3 + 7’) | | |
| Others | 14 | (8.1%) |
| No treatment/unknown | 6 | (3.5%) |

Allogeneic Stem cell
transplantation (allo-SCT)

| | | |
|---|---|---|
| Yes (%) | 41/172 | (23.8%) |
| At CR (%) | 20/41 | (48.8%) |
| At relapse post-CR (%) | 18/41 | (43.9%) |
| In primary refractory | 3/41 | (7.3%) |
| status (%) | | |
| No (%) | 131/172 | (76.2%) |

Mutations

| | | |
|---|---|---|
| FLT3-ITD | 47 | (27.3%) |
| NPM1 | 47 | (27.3%) |

CEBPA

| Double (%), single (%) | 15 (9.8%), 12 (9.2%) | |
|---|---|---|
| In-frame bZIP | 20 | (11.6%) |
| mutation (%) | | |
| DNMT3A | 44 | (25.6%) |

Abbreviations: BM, bone marrow; WBC, white blood cell; FAB, French-American-British classification; MRC, Medical Research Council classification; CR, complete remission; ITD, internal tandem duplication; bZIP, basic leucine zipper domain.

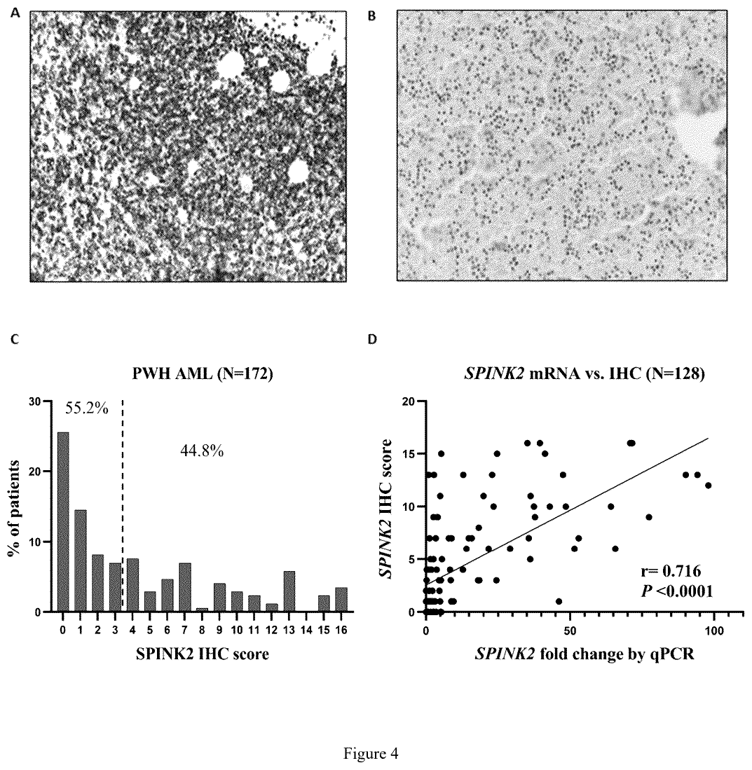

SPINK2 IHC staining in leukemic blasts is consistently cytoplasmic (FIGS. 4A-B) and is quantified using a composite IHC score based on the percentage of stained blasts and the intensity of the staining (range: 0-16, median: 3) (FIG. 4C). Furthermore, SPINK2 protein expression strongly correlated with its mRNA levels assessed by qPCR in a subset of 128 adult patients with available RNA (r=0.716, P<0.0001) (FIG. 4D).

### 1.3.Mutational and Clinicopathological Associations of SPINK2 in AML

Univariate clinicopathological analyses are initially performed by dichotomization at the median SPINK2 IHC score of ‘3’ since this cut-off exhibited strongest association with adverse event-free survival (EFS) and overall survival (OS) (Table S2).

TABLE S2
Supplementary Table S2. Determination of SPINK2 IHC cut-off with
strongest prognostic implications in the adult AML cohort.

| | Median | 5-yr | | |
|---|---|---|---|---|
| | survival | survival, % | | |
| | (SPINK2^{high} | (SPINK2^{low} | Logrank | |
| | vs. | vs. | Hazard Ratio | Logrank |
| Cut-off | SPINK2^{low}) | SPINK2^{high}) | (95% C.I.) | P-value |
| q4 vs. | OS: 15.5 vs. | OS: 44.5 | HR: 1.749 | 0.019 |
| q3-q2-q1 | 28 months | vs. 26.3 | (0.998-3.066) | |
| | EFS: 9.5 vs. | EFS: 30.8 | HR: 1.581 | 0.0397 |
| | 14 months | vs. 21.1 | (0.942-2.654) | |
| q4-q3 vs. | OS: 15 vs. | OS: 51.2 | HR: 2.064 | 0.0007 |
| q2-q1 | 74 months | vs 25.3 | (1.309-3.255) | |
| | EFS: 8 vs. | EFS: 37.2 | HR: 1.966 | 0.0005 |
| | 18 months | vs. 16.6 | (1.292-2.990) | |
| q4-q3-q2 | OS: 19 vs. | OS: 43.1 | HR: 1.348 | 0.2309 |
| vs. q1 | 28 months | vs 38.6 | (0.845-2.149) | |
| | EFS: 11 vs. | EFS: 33.9 | HR: 1.293 | 0.2489 |
| | 14.5 months | vs. 25.4 | (0.844-1.980) | |

Abbreviations: OS, overall survival;
EFS, event-free survival;
HR, hazard ratio

SPINK2^{high} is thus defined as score>3, and SPINK2^{low} as score≤3. SPINK2^{high} status is found in 77/172 (44.8%) patients, while SPINK2^{low} status is found in 95/172 (55.2%) patients. SPINK2^{high} status associated significantly with the intermediate-risk (IR) subgroup, both by cytogenetics (P=0.014) and by the European LeukemiaNet (ELN) 2022 classification (P=0.009). Further significant associations are found with the normal karyotype (NK) (P=0.019), NPM1 (P<0.0001) and DNTM3A (P=0.022) mutations, including with mutational combinations, such as NPM1+/DNMT3A+ (P=0.007) and NPM1+/FLT3-ITD+ (P=0.017). SPINK2^{high} status inversely associated with t (8;21) translocation (P<0.001), and CEBPA mutations in the basic-region leucine zipper motif (bZIP) (P=0.001) (Table 1). Other commonly recurring myeloid mutations identified by NGS, including high-risk mutations such as TP53, RUNX1, ASXL1, showed no significant correlation with SPINK2 status, and are listed in Table 1. Moreover, analysis of available cytogenetic and mutational data of 982 patients from 3 adult AML cohorts (TCGA-LAML, OHSU and Verhaak) largely confirmed the observations from Table S3.

TABLE 1

| | | | |
|---|---|---|---|
| | High SPINK2 | Low SPINK2 | |
| Characteristic | (n = 77) | (n = 95) | P-value |

Sex

| | | | | | |
|---|---|---|---|---|---|
| Male | 41 | (53.3%) | 48 | (50.5%) | 0.76 |
| Female | 36 | (46.7%) | 47 | (49.5%) | |

Age, years

| | | | | | |
|---|---|---|---|---|---|
| Median (range) | 54 | (20-75) | 51 | (18-86) | 0.23 |

Hb level, g/dl

| | | | | | |
|---|---|---|---|---|---|
| Median (range) | 8.5 | (3-13.6) | 7.8 | (2.9-12.9) | 0.16 |

Bone Marrow blast, %

| | | | | | |
|---|---|---|---|---|---|
| Median, (range) | 69 | (11-98) | 69 | (12-98) | 0.65 |

WBC level, ×10⁹/L

| | | | | | |
|---|---|---|---|---|---|
| Median, (range) | 28.1 | (1.3-517) | 19.8 | (0.9-330.4) | 0.18 |

Platelets, ×10⁹/L

| | | | | | |
|---|---|---|---|---|---|
| Median, (range) | 67 | (4-748) | 43 | (2-247) | <0.001 |

FAB classification

| | | | | | |
|---|---|---|---|---|---|
| M0 | 1/48 | (2.1%) | 4/68 | (5.9%) | 0.40 |
| M1 | 11/48 | (22.9%) | 21/68 | (30.9%) | 0.40 |
| M2 | 8/48 | (16.7%) | 17/68 | (25.0%) | 0.36 |
| M4(incl. M4Eo) | 11/48 | (22.9%) | 13/68 | (19.1%) | 0.65 |
| M5 | 16/48 | (33.3%) | 11/68 | (16.2%) | 0.04 |
| M6 | 1/48 | (2.1%) | 2/68 | (2.9%) | 0.99 |
| Unclassified | 29/77 | (37.6%) | 27/95 | (28.4%) | — |

AML type

| | | | | | |
|---|---|---|---|---|---|
| De novo | 71 | (92.2%) | 85 | (89.5%) | 0.61 |
| Secondary/t-AML | 6 | (7.8%) | 10 | (10.5%) | |

MRC Cytogenetic Risk

| | | | | | |
|---|---|---|---|---|---|
| Favourable | 5 | (6.5%) | 19 | (20.7%) | 0.014 |
| Intermediate | 64 | (83.1%) | 61 | (66.3%) | 0.014 |
| Adverse | 8 | (10.4%) | 12 | (13.0%) | 0.64 |

| | | | |
|---|---|---|---|
| Unclassified | — | 3 | — |

ELN 2022 risk

| | | | | | |
|---|---|---|---|---|---|
| Favorable | 19/73 | (26.0%) | 39/92 | (42.4%) | 0.033 |
| Intermediate | 34/73 | (46.6%) | 24/92 | (26.1%) | 0.009 |
| Adverse | 20/73 | (27.4%) | 29/92 | (31.5%) | 0.61 |

Cytogenetics

| | | | | | |
|---|---|---|---|---|---|
| Normal | 51 | (66.2%) | 44 | (47.8%) | 0.019 |
| t(8; 21) | 0 | (0.0%) | 14 | (15.2%) | <0.001 |
| inv(16) | 5 | (6.5%) | 4 | (4.4%) | 0.73 |
| Complex | 5 | (6.5%) | 5 | (5.4%) | 0.99 |
| Others | 12 | (15.6%) | 20 | (21.7%) | 0.33 |

| | | | | |
|---|---|---|---|---|
| Unknown | — | 3 | (3.2%) | — |

Mutations

| | | | | | |
|---|---|---|---|---|---|
| FLT3-ITD | | | | | |
| NPM1 | 26/77 | (33.8%) | 21/95 | (22.1%) | 0.12 |
| CEBPA bZIP | 33/77 | (42.9%) | 14/95 | (14.7%) | <0.0001 |
| DNMT3A | 2/74 | (2.7%) | 18/95 | (19.0%) | 0.001 |
| NPM1+/DNMT3A+ | 26/74 | (35.1%) | 18/95 | (19.0%) | 0.022 |
| NPM1+/FLT3-ITD+ | 17/74 | (23.0%) | 7/95 | (7.4%) | 0.007 |
| NPM1+/FLT3− | 20/77 | (26.0%) | 11/95 | (11.6%) | 0.017 |
| ITD+/DNMT3A+ | 9/74 | (12.2%) | 6/95 | (6.3%) | 0.275 |
| TP53 | 1/69 | (1.5%) | 2/86 | (2.3%) | 0.99 |
| RUNX1 | 8/69 | (11.6%) | 12/86 | (14.0%) | 0.81 |
| ASXL1 | 4/69 | (5.8%) | 4/86 | (4.7%) | 0.99 |
| BCOR | 2/69 | (2.9%) | 2/86 | (2.3%) | 0.99 |
| EZH2 | 1/69 | (1.5%) | 3/86 | (3.5%) | 0.63 |
| SF3B1 | 0/69 | (0.0%) | 1/86 | (1.2%) | 0.99 |
| SRSF2 | 4/69 | (5.8%) | 4/86 | (4.7%) | 0.99 |
| STAG2 | 1/69 | (1.5%) | 6/86 | (7.0%) | 0.13 |
| U2AF1 | 0/69 | (0.0%) | 2/86 | (2.3%) | 0.50 |
| ZRSR2 | 2/69 | (2.9%) | 0/86 | (0.0%) | 0.20 |

Hb, hemoglobin;
WBC, white blood cell count;
FAB, French-American-British Classification;
MRC, Medical Research Council;
ELN, European LeukemiaNet;
ITD, internal tandem duplication;
bZIP, basic-region leucine zipper motif

TABLE S3

| | TCGA-LAML (N = 173) | | | OHSU-BEAT-AML (N = 392) | | | Verhaak (N = 417) | | |
|---|---|---|---|---|---|---|---|---|---|
| | High | Low | | High | Low | | High | Low | |
| Mutations & | SPINK2 | SPINK2 | P | SPINK2 | SPINK2 | P | SPINK2 | SPINK2 | P |
| Cytogenetics | n = 87 | n = 86 | value^{‡} | n = 196 | n = 196 | value^{‡} | n = 208 | n = 209 | value^{‡} |
| Mutations | | | | | | | | | |
| NPM1 | 31(35.63%) | 17(19.77%) | 0.027 | 62(31.6%) | 32(16.3%) | 0.0006 | 89(42.8%) | 48(23.0%) | <0.0001 |
| FLT3-ITD | 25(28.74%) | 12(13.95%) | 0.025 | 59(30.1%) | 28(14.3%) | 0.0002 | 81(38.9%) | 36(17.2%) | <0.0001 |
| CEBPAdm | 1(1.15%) | 4(4.65%) | 0.211 | N/A | N/A | N/A | 6(2.9%) | 17(8.1%) | 0.029 |
| DNMT3A | 28(32.18%) | 15(17.44%) | 0.034 | 54(27.6%) | 28(14.3%) | 0.002 | N/A | N/A | N/A |
| Cytogenetics^{#} | | | | | | | | | |
| Normal | 47(55.3%) | 33(38.8%) | 0.045 | 93(51.7%) | 62(35.6%) | 0.003 | 104(55.6%) | 75(38.9%) | 0.001 |
| t (8; 21) | 0(0.00%) | 7(8.24%) | 0.014 | 0(0.00%) | 8(4.6%) | 0.003 | 0(0.0%) | 35(18.1%) | <0.0001 |
| inv (16) | 3(3.61%) | 7(8.24%) | 0.329 | 8(4.4%) | 17(9.8%) | 0.062 | 10(5.4%) | 23(11.9%) | 0.028 |
| Cytogenetic risk^{$} | | | | | | | | | |
| Favorable | 8(9.4%) | 24(28.2%) | 0.003 | N/A | N/A | N/A | 11(5.4%) | 63(31.0%) | <0.0001 |
| Intermediate | 58(68.2%) | 43(50.6%) | 0.028 | N/A | N/A | N/A | 146(71.6%) | 101(49.8%) | <0.0001 |
| Adverse | 19(22.4%) | 18(21.2%) | >0.99 | N/A | N/A | N/A | 47(23.0%) | 39(19.2%) | 0.396 |
| ELN 2022 risk* | | | | | | | | | |
| Favorable | 23(26.4%) | 41/85(48.2%) | 0.004 | N/A | N/A | N/A | N/A | N/A | N/A |
| Intermediate | 33(37.9%) | 15/85(17.5%) | 0.004 | N/A | N/A | N/A | N/A | N/A | N/A |
| Adverse | 31(35.6%) | 29/85(34.1%) | 0.87 | N/A | N/A | N/A | N/A | N/A | N/A |

### 1.4.Higher SPINK2 Expression Contributes to Therapy Resistance in AML

Survival and treatment-response analyses are initially performed on a subgroup of 137 patients that included only de novo AML patients treated on standard induction regimens with daunorubicin and cytarabine backbone (DA 3+7). Complete remission (CR) is achieved by 112/137 (81.8%) patients after one or more induction courses, while 25/137 (18.2%) patients are non-responsive (NR). SPINK2^{high} patients have lower CR rates vs. SPINK2^{low} patients irrespective of the number of inductions (73.3% vs 88.3%, P=0.028). Of note, non-response to 1^{st} induction (NR1) is more frequent in these patients (51.7% vs 33.8%, P=0.038). Indeed, patients with NR1 have higher median SPINK2 scores vs. patients with CR at 1^{st} induction (CR1) (5 vs 1.5, P=0.025).

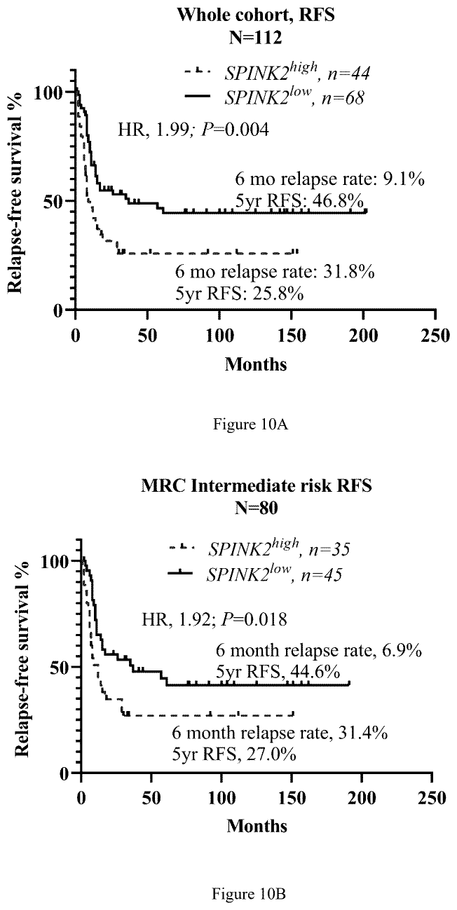

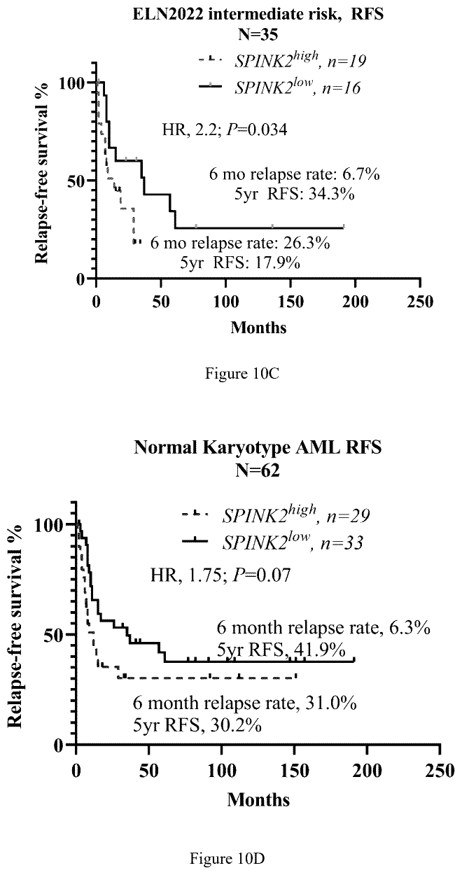

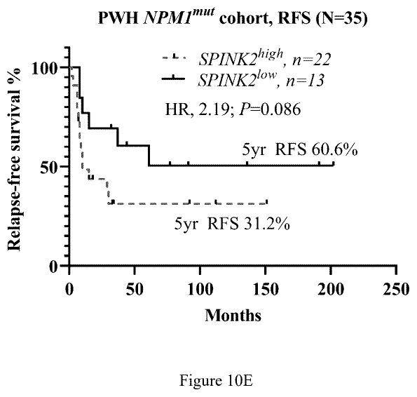

Median relapse-free survival (RFS) of patients achieving CR is inferior in SPINK2^{high} vs SPINK2^{low} patients (9 vs. 37 months; P=0.004), with the SPINK2^{high} subgroup having higher relapse incidence within 6 months (31.8% vs. 9.1%, P=0.004) (FIG. 10A).

The following subgroups are analyzed due to their significant association with SPINK2 expression: IR by cytogenetics and ELN 2022, NK-AML and NPM1^{mut} (Table 2). In most subgroups, high SPINK2 expression is linked to lower CR rates and higher NR1 rates. Relapse risk is also elevated, achieving statistical significance in IR groups while demonstrating significant trends in NK-AML and NPM1^{mut} subgroups. Survival curves for RES can be found in FIGS. 10A-E.

TABLE 2

| | High | | Low | | |
|---|---|---|---|---|---|
| Factor | SPINK2 | | SPINK2 | | P-value |
| Whole cohort (N = 137) | N = 60 | | N = 77 | | |
| Response to induction | | | | | |
| CR | 73.3% | | 88.3% | | 0.028 |
| NR1 | 51.7% | | 33.8% | | 0.038 |
| Relapse^{‡} after CR | | | | | |
| Median RFS | 9 | months | 37 | months | 0.004 |
| 6-month relapse rate | 31.8% | | 9.1% | | |
| 5 yr RFS | 25.8% | | 46.8% | | |
| Intermediate cytogenetic | N = 51 | | N = 50 | | |
| risk (N = 101) | | | | | |
| Response to induction | | | | | |
| CR | 68.6% | | 90.0% | | 0.01 |
| NR1 | 66.7% | | 37.5% | | 0.005 |
| Relapse^{‡} after CR | | | | | |
| Median RFS | 12 | months | 37 | months | 0.018 |
| 6-month relapse rate | 31.4% | | 6.9% | | |
| 5 yr RFS | 27.0% | | 44.6% | | |
| Intermediate risk ELN | N = 28 | | N = 19 | | |
| 2022 (N = 47) | | | | | |
| Response to induction | | | | | |
| CR | 67.9% | | 84.2% | | 0.31 |
| NR1 | 67.9% | | 21.1% | | 0.003 |
| Relapse^{‡} after CR | | | | | |
| Median RFS | 14 | months | 37 | months | 0.034 |
| 6-month relapse rate | 26.3% | | 6.7% | | |
| 5 yr RFS | 17.9% | | 34.3% | | |
| Normal karyotype (N = 76) | N = 40 | | N = 36 | | |
| Response to induction | | | | | |
| CR | 72.5% | | 91.7% | | 0.040 |
| NR1 | 55.0% | | 27.8% | | 0.021 |
| Relapse^{‡} after CR | | | | | |
| Median RFS | 12 | months | 35 | months | 0.07 |

| 6-month relapse rate | 31.0% | | 6.2% | |
|---|---|---|---|---|
| 5 yr RFS | 30.2% | | 41.9% | |
| NPM1^{mut} (N = 46) | N = 33 | | N = 13 | |
| Response to induction | | | | |
| CR | 75.8% | | 100% | 0.08 |
| NR1 | 48.5% | | 15.4% | 0.049 |
| Relapse^{‡} after CR | | | | |
| Median RFS | 14 | months | Unreached | 0.095 |

| | | |
|---|---|---|
| 6-month relapse rate | 28.0% | 0.0% |
| 5 yr RFS | 35.5% | 50.5% |

^{‡}Relapse rates are calculated only for patients who achieved CR.
Abbreviations: CR, complete response achieved irrespective of number of inductions;
NR1, non-response at 1^{st} induction;
RFS: relapse-free survival

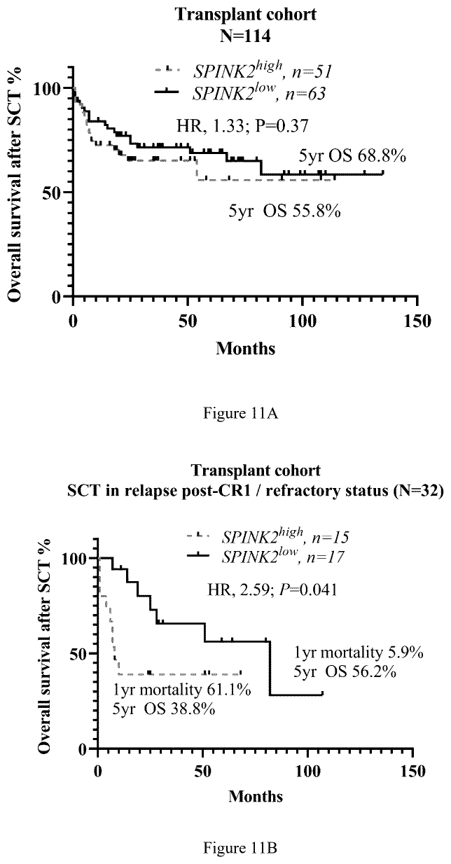

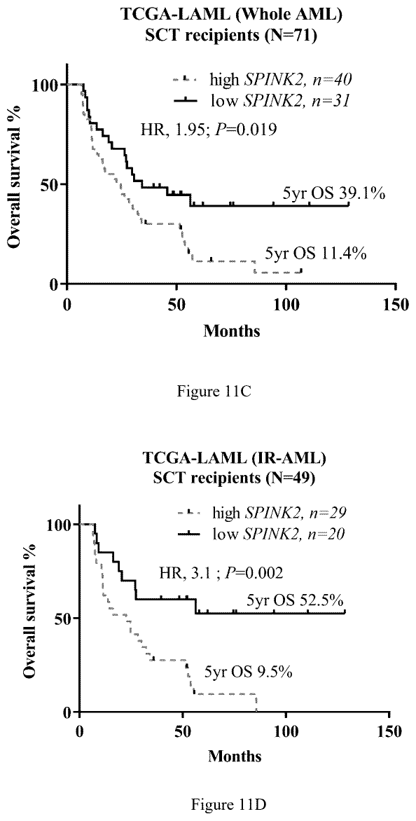

The association of SPINK2 expression with outcome after SCT is next investigated. In the cohort, 37 patients received SCT treatment. To ascertain the association of SPINK2 and SCT outcome, an additional 77 SCT recipients are recruited from partner hospitals, and their diagnostic BM specimens are examined for SPINK2 protein expression. In this combined transplant cohort of 114 patients, SPINK2^{high} status does not significantly affect OS after SCT receipt (5yr OS: 55.8% vs. 68.8%, P=0.37) and this is further illustrated in FIG. 11A wherein KM survival curve comparing post-SCT-OS between patients with higher and lower median SPINK2 expression of the combined transplant cohort, N=114. Median post-SCT OS (high vs. low SPINK2): both unreached. However, 1yr-mortality after SCT is significantly increased in SPINK2^{high} patients who received SCT in relapse after CR1 or in refractory status (61.1% vs 5.9%, P=0.041) and this is illustrated in FIG. 11B wherein KM survival curve comparing post-SCT OS between patients with higher and lower median SPINK2 expression who received SCT in relapse after CR or in refractory status. Median post-SCT OS (high vs. low SPINK2): 8 vs. 82 months.

In the PWH combined transplant cohort, the OS after SCT is calculated as the survival time elapsed from receipt of SCT until last follow-up or death. In the TCGA-LAML cohort, the OS after SCT is not available. Therefore, the total OS, i.e., survival time from date of diagnosis until loss of follow-up or death, is calculated.

In the TCGA-LAML cohort, SCT-recipients (N=71) with higher median SPINK2 mRNA has worse 5yr OS, both in the whole cohort (11.4% vs. 39.1%, P=0.019) and the IR subgroup (9.5% vs. 52.5%, P=0.002) and these are illustrated in FIGS. 11C-D wherein (C) illustrates KM survival curve comparing OS between SCT-recipient patients with higher and lower median SPINK2 expression of the TCGA-LAML heterogeneous cohort, N=71. Median OS (high vs low SPINK2): 23.6 vs 34.4 months and (D) KM survival curve comparing OS between SCT-recipient patients with higher and lower median SPINK2 expression of the TCGA-LAML IR-AML cohort, N=49. Median OS (high vs. low SPINK2): 22.6 months vs. unreached. The logrank P-value and logrank hazard ratio (HR) are used for comparison of groups. Collectively, these findings suggest that SPINK2 plays an important role in protection of leukemic cells against current antileukemic therapy, thereby increasing risk of relapse.

### 1.5.High SPINK2 Expression Refines Current Prognostic Stratification and is an Independent Adverse Prognostic Marker

Survival analyses are initially performed on the whole cohort (N=137) which comprised only de novo AML patients treated on the DA 3+7 protocol, and subsequently on specific subgroups which have significant associations with SPINK2 expression: IR risk (by cytogenetics and ELN 2022 criteria), NK-AML and NPM1^{mut}-AML. The TCGA-LAML cohort is also analysed.

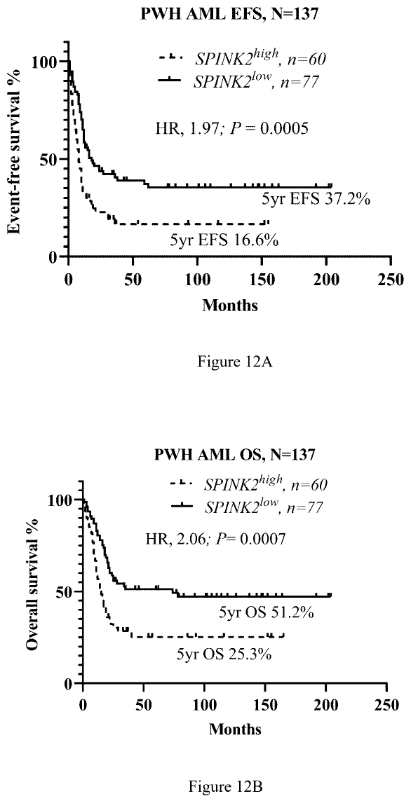

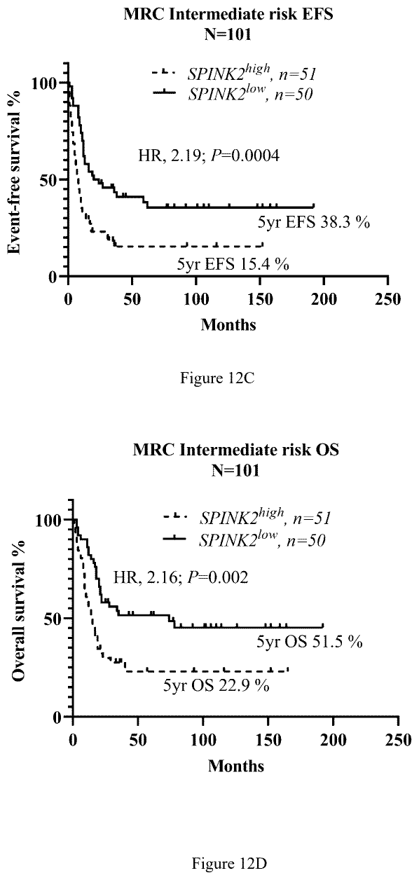

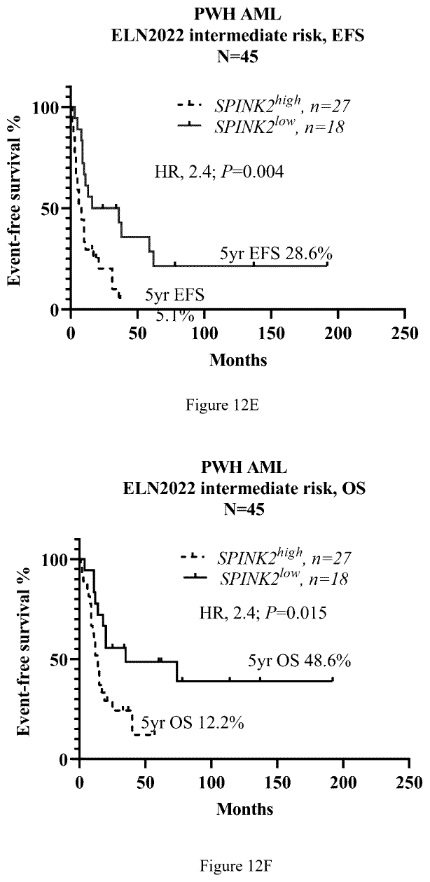

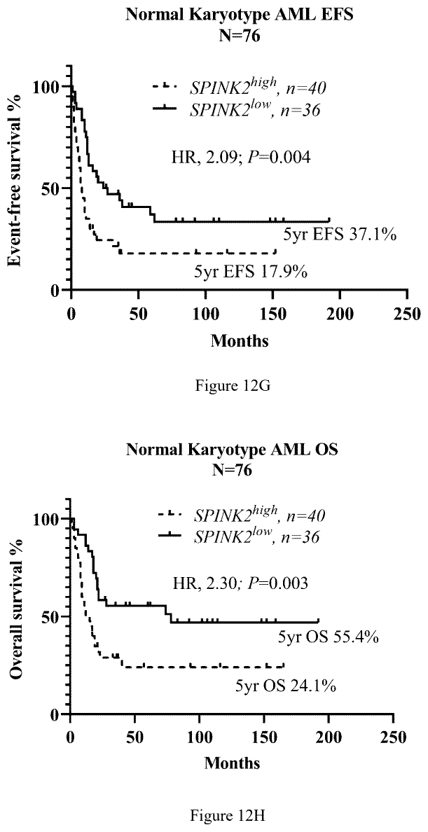

Univariate Kaplan-Meier analyses showed that SPINK2^{high} status associated significantly with inferior EFS and OS in all aforementioned subgroups as illustrated in FIGS. 12A-J. FIGS. 12 (A-B) illustrate survival curves of the heterogeneous cohort (N=137) for EFS (A) and OS (B). Median survival in SPINK2^{high} vs. SPINK2^{low} groups: EFS, 8 vs 18 months; OS, 15 vs. 74 months; (C-D) Survival curves of the cytogenetic IR cohort (N=101) for EFS (C) and OS (D). Median survival in SPINK2^{high} vs. SPINK2^{low} groups: EFS, 8 vs 22 months; OS, 15 vs. 74 months; (E-F) Survival curves of the ELN 2022 IR cohort (N=45) for EFS (E) and OS (F). Median survival in SPINK2^{high} vs. SPINK2^{low} groups: EFS, 8 vs 26 months; OS, 14 vs. 35 months; (G-H) Survival curves of the NK-AML cohort (N=76) for EFS (G) and OS (H). Median survival in SPINK2^{high} vs. SPINK2^{low} groups: EFS, 8 vs 25.5 months; OS, 13.5 vs. 78 months; (I-J) Survival curves of the NPM1^{mut} cohort (N=46) for EFS (I) and OS (J). Median survival in SPINK2^{high} vs. SPINK2^{low} groups: EFS, 9 months vs unreached; OS, 11 months vs. unreached. Survival proportions are compared using the logrank P-value and logrank hazard ratio (HR).

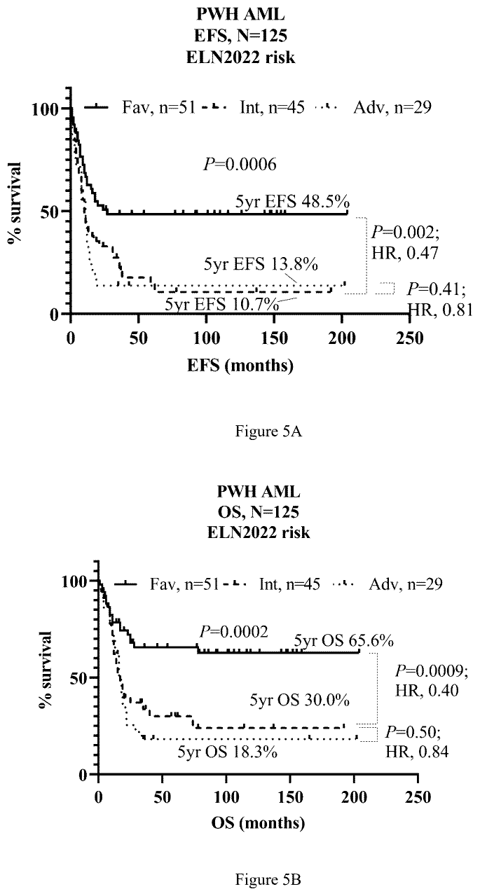

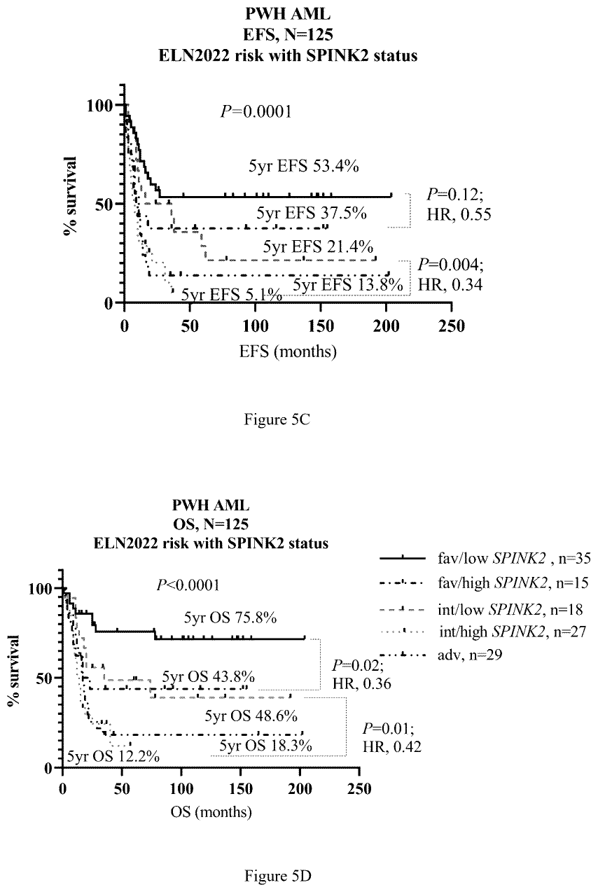

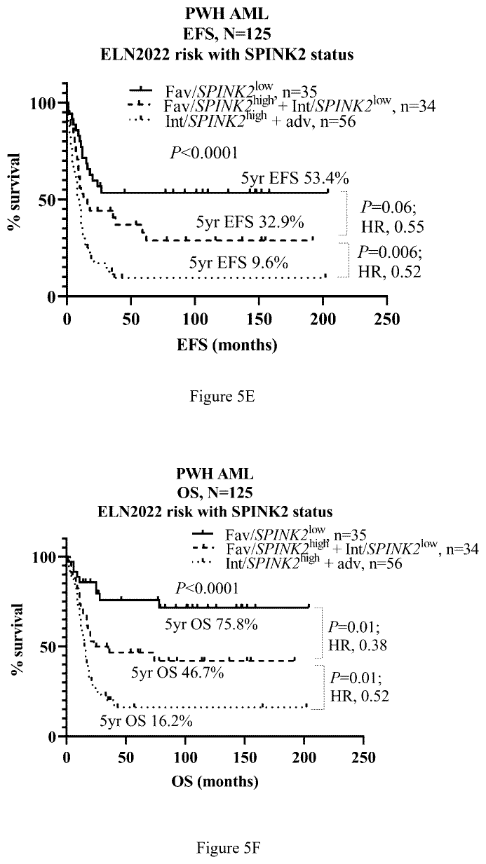

Additionally, SPINK2 expression could identify high-risk patients among the ELN 2022 favorable-risk and intermediate risk cohorts (FIG. 5C, D). Incorporation of SPINK2 IHC status with ELN 2022 criteria could thus significantly refine patient risk stratification (FIGS. 5A, B, E, F). It is further illustrated in FIG. 5 wherein (A, B) Kaplan-Meier (KM) survival curves for EFS (A) and OS (B) based upon ELN 2022 risk only. (C, D) KM curves for EFS (C) and OS (D) based upon ELN 2022 risk with incorporation of SPINK2 IHC status. (E, F) KM curves for EFS (E) and OS (F) based upon ELN 2022 risk with incorporation of SPINK2 IHC status and combination of indicated categories. Survival proportions are compared using the logrank P-value and logrank hazard ratio (HR).

Table S4 is baseline characteristics of additional 77 BMT patients.

TABLE S4
Supplementary Table S4. Baseline characteristics
of the 77 additional adult AML patients recruited
for analysis of SPINK2 and SCT outcomes

| | |
|---|---|
| Characteristic | Value |

Age

| | | |
|---|---|---|
| Range (median), yrs. | 18-60 | (45) |
| Gender: Male (%) | 36 | (46.8%) |
| Female (%) | 41 | (53.2%) |

Cytogenetics subgroups

| | | |
|---|---|---|
| Normal (%) | 37 | (48.1%) |
| Non-Normal (%) | 39 | (50.6%) |
| Unknown (%) | 1 | (1.3%) |

Response to therapy

| | |
|---|---|
| CR1 (%)/NR1 (%) | 67 (87%)/10 (13%) |
| Relapse (%)/No relapse | 30 (39%)/47 (61%) |

Allogeneic Stem cell
transplantation (allo-SCT) stage

| | | |
|---|---|---|
| At CR1 (%) | 63 | (81.8%) |
| At relapse post-CR1 (%) | 14 | (18.2%) |

Median follow-up after SCT

| | | |
|---|---|---|
| Range (median), months. | 1-114 | (31) |

Abbreviations: CR1, complete remission at 1st induction;
NR1, non-responsive at 1st induction

Importantly, multivariate analyses in the cohort highlighted the poor prognostic effect of SPINK2^{high} status on RFS (HR: 1.89, 95% C.I.: 1.12-3.15, P=0.015), EFS (HR: 2.08, 95% C.I.: 1.31-3.32, P=0.002) and OS (HR: 2.45, 95% C.I.: 1.48-4.07, P<0.001) independent of age, ELN 2022 risk status and CR1, including SCT given in CR (Table 3). In the NPM1^{mut} subgroup, SPINK 2^{high} status predicted poor RFS (HR: 3.52, 95% C.I.: 1.23-11.72, P=0.027), EFS (HR: 5.11, 95% C.I.: 1.91-16.65, P=0.003) and OS (HR: 5.55, 95% C.I.: 1.89-21.32, P=0.005) independent of age, concomitant FLT3 and DNMT3A mutational status. Table 3 below summarizes the multivariate analysis for OS, EFS and RFS.

TABLE 3

| | Covariates | | | | | | | | |
|---|---|---|---|---|---|---|---|---|---|
| | OS | | | EFS | | | RFS^{§} | | |
| | HR | 95% C.I. | P-value | HR | 95% C.I. | P-value | HR | 95% C.I. | P-value |
| Whole cohort^{‡} | | | | | | | | | |
| N = 125^{¶} | | | | | | | | | |
| Age ≥60 yrs | 1.29 | 0.68-2.36 | 0.416 | / | / | / | / | / | / |
| SPINK2^{high} | 2.45 | 1.48-4.07 | <0.001 | 2.08 | 1.31-3.32 | 0.002 | 1.89 | 1.12-3.15 | 0.015 |
| CR1 | 0.40 | 0.24-0.67 | <0.001 | 0.33 | 0.21-0.52 | <0.001 | / | / | / |
| SCT in CR | 0.11 | 0.02-0.37 | 0.0023 | 0.15 | 0.04-0.36 | <0.001 | 0.11 | 0.02-0.37 | 0.003 |
| DNMT3A | 1.20 | 0.72-1.96 | 0.479 | 1.18 | 0.73-1.87 | 0.490 | 1.602 | 0.91-2.73 | 0.090 |
| ELN 2022 | 1.78 | 1.02-3.02 | 0.037 | 1.86 | 0.94-3.43 | 0.060 | 2.16 | 1.21-3.73 | 0.007 |
| adv | | | | | | | | | |
| IDH2 | 2.33 | 1.18-4.31 | 0.010 | 1.58 | 0.93-2.59 | 0.080 | / | / | / |
| NPM1^{mut †} | | | | | | | | | |
| N = 42^{¶} | | | | | | | | | |
| Age ≥60 yrs | 9.10 | 2.36-34.39 | 0.001 | 7.53 | 2.01-27.45 | 0.002 | 3.58 | 0.92-12.13 | 0.046 |
| SPINK2^{high} | 5.55 | 1.89-21.32 | 0.005 | 5.11 | 1.91-16.65 | 0.003 | 3.52 | 1.23-11.72 | 0.027 |
| FLT3-ITD | 2.54 | 0.94-8.18 | 0.085 | 3.9 | 1.37-11.94 | 0.017 | 2.47 | 0.88-7.84 | 0.100 |
| DNMT3A | 0.81 | 0.34-1.99 | 0.635 | 1.10 | 0.49-2.57 | 0.824 | 3.12 | 1.20-9.65 | 0.029 |

CR1: Complete remission at 1^{st} induction,
SCT in CR: stem cell transplantation administered after achieving complete remission,
ELN 2022 adv: ELN 2022 adverse risk,
ITD: internal tandem duplication,
HR: hazard ratio,
C.I.: confidence interval
^{§}For RFS analysis, only patients eventually achieving CR are included in the analysis in all cohorts (whole, N = 108; NPM1^{mut}, N = 38)
^{‡}The covariates included in the multivariate analyses are those which demonstrated significant associations (P < 0.05) with in univariate survival analyses (Tables S5A-B)
^{†} The covariates included in NPM1 analysis are those which are part of ELN 2022 criteria (FLT3-ITD) and generally associated with poor prognosis in NPM1^{mut} patients (age, DNMT3A)
^{¶}Only those patients are included who have complete cytogenetic and mutational data which allowed for assignment to an ELN 2022 risk category

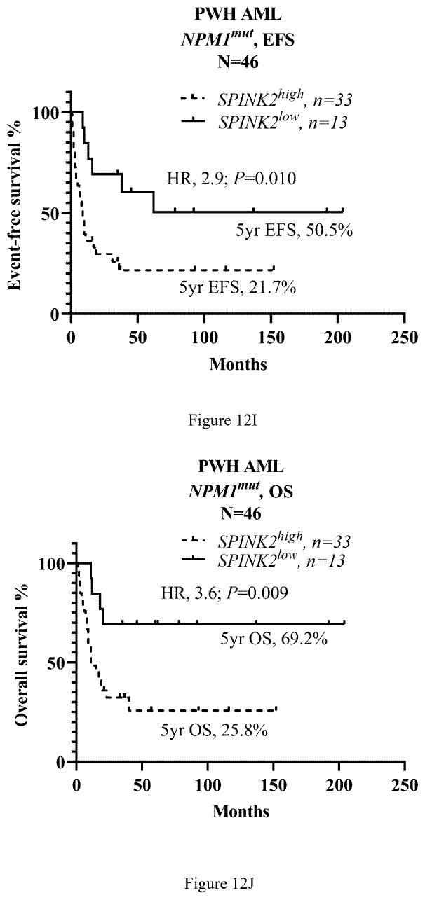

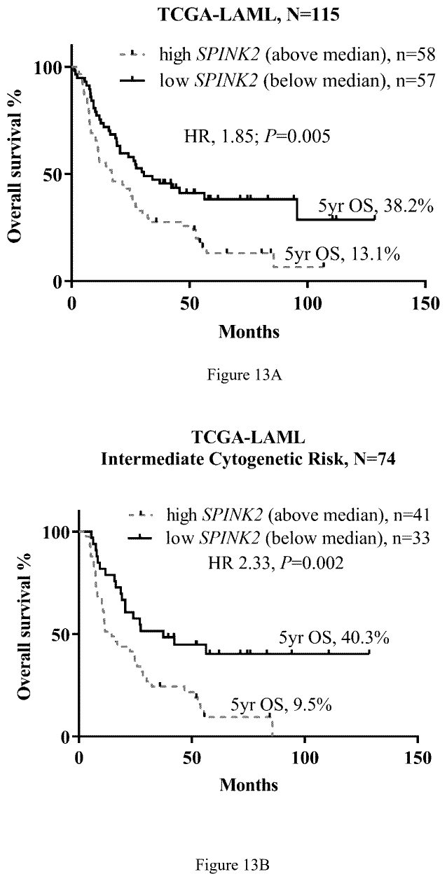

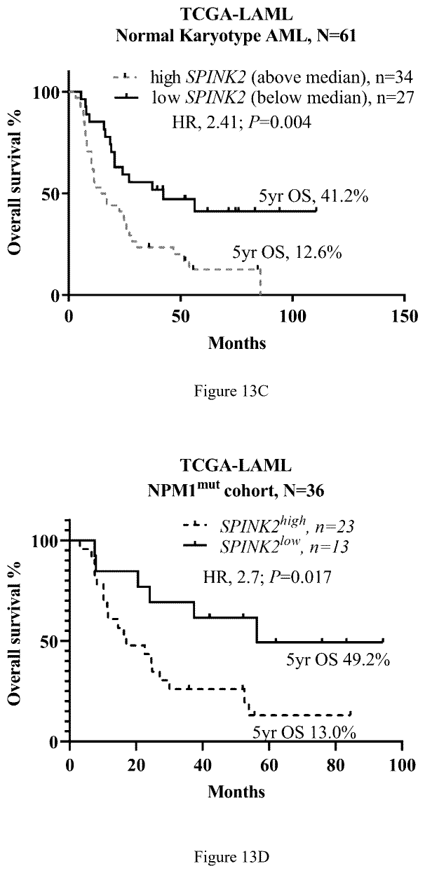

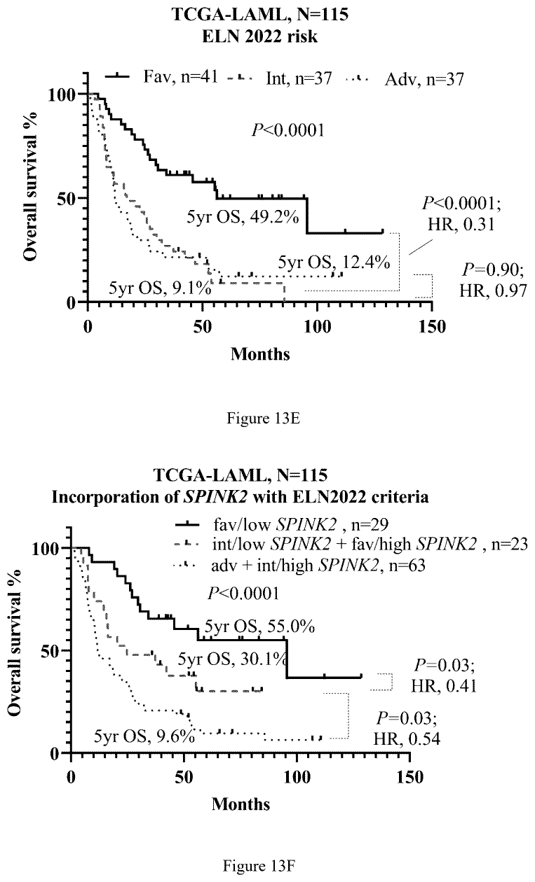

These findings could also be observed in patients of the TCGA-LAML cohort, who have received standard DA 3+7 based induction regimens (N=115). Univariate survival analyses demonstrated that higher SPINK2 mRNA expression is associated with inferior OS in the whole cohort, and subgroups such as cytogenetic IR, NK-AML and NPM1^{mut}. SPINK2 expression could significantly refine risk stratification by ELN 2022 criteria and is an independent prognostic factor (FIGS. 13A-F, Tables S6).

TABLE S6

| | Univariate | | | | Multivariate | | | |
|---|---|---|---|---|---|---|---|---|
| | P- | | 95.0% CI for HR^{$} | | P- | | 95.0% CI for HR^{#} | |
| Factor | value^{$} | HR^{$} | Lower | Upper | value^{#} | HR^{#} | Lower | Upper |
| Age >60 yrs | 0.002 | 2.016 | 1.275 | 3.135 | 0.002 | 2.074 | 1.307 | 3.329 |
| High SPINK2 | 0.005 | 1.859 | 1.209 | 2.892 | 0.054 | 1.547 | 0.997 | 2.425 |
| WBC >16 | 0.375 | 0.821 | 0.527 | 1.264 | / | / | / | / |
| DNMT3A | 0.026 | 1.703 | 1.051 | 2.688 | 0.01 | 1.914 | 1.156 | 3.099 |
| ELN 2022 | <0.001 | 0.318 | 0.187 | 0.518 | / | / | / | / |
| favorable risk | | | | | | | | |
| ELN 2022 | 0.01 | 1.783 | 1.136 | 2.763 | / | / | / | / |
| intermediate risk | | | | | | | | |
| ELN 2022 | 0.008 | 1.823 | 1.161 | 2.819 | 0.007 | 1.88 | 1.181 | 2.951 |
| adverse risk | | | | | | | | |
| Receipt of SCT | 0.32 | 0.802 | 0.519 | 1.239 | / | / | / | / |

Supplementary Table S6. Univariate & multivariate survival analysis for OS in the TCGA-LAML cohort.
^{$}P-value, Hazard ratio (HR) with 95% CI calculated using Cox regression survival analysis
^{#}P-value and Hazard ratio (HR) of the multivariate Cox regression analysis
WBC, white blood cell count;
SCT, stem cell transplantation;
ELN, European LeukemiaNet

Additionally, SPINK2 expression remained an independent prognostic factor for OS in pairwise multivariate Cox analyses comparing SPINK2 expression and three previously published LSC gene expression signatures, particularly in IR and NK subgroups (Table S7).

TABLE S7

| | Whole, N = 115 | | | Intermediate-risk (IR), N = 74 | | | Normal karyotype (NK), N = 61 | | |
|---|---|---|---|---|---|---|---|---|---|
| Predictor | HR^{$} | 95% CI^{$} | P-value | HR^{$} | 95% CI^{$} | P-value^{$} | HR^{$} | 95% CI^{$} | P-value^{$} |
| SPINK2 | 1.246 | 0.7625-2.055 | 0.383 | 1.921 | 1.057-3.582 | 0.035 | 2.125 | 1.096-4.256 | 0.028 |
| LSC17 score | 2.347 | 1.437-3.862 | <0.001 | 1.738 | 0.981-3.108 | 0.059 | 1.486 | 0.785-2.816 | 0.221 |
| (Ng et al) | | | | | | | | | |
| SPINK2 | 1.752 | 1.133-2.738 | 0.012 | 2.109 | 1.201-3.823 | 0.011 | 2.115 | 1.119-4.157 | 0.024 |
| Gentles et al | 0.691 | 0.439-1.072 | 0.104 | 0.608 | 0.352-1.050 | 0.073 | 0.584 | 0.315-1.090 | 0.088 |
| SPINK2 | 1.854 | 1.204-2.888 | 0.006 | 2.413 | 1.391-4.326 | 0.002 | 2.46 | 1.336-4.727 | 0.005 |
| Eppert et al | 1.035 | 0.674-1.592 | 0.8745 | 0.897 | 0.523-1.526 | 0.69 | 0.929 | 0.504-1.681 | 0.8097 |

Supplementary Table S7. Multivariate pairwise Cox analysis for OS in the TCGA-LAML cohort comparing SPINK2 expression and published LSC gene expression signatures (Ng, Gentles and Eppert)
^{$}P-value, Hazard ratio (HR) with 95% CI calculated using Cox regression analysis

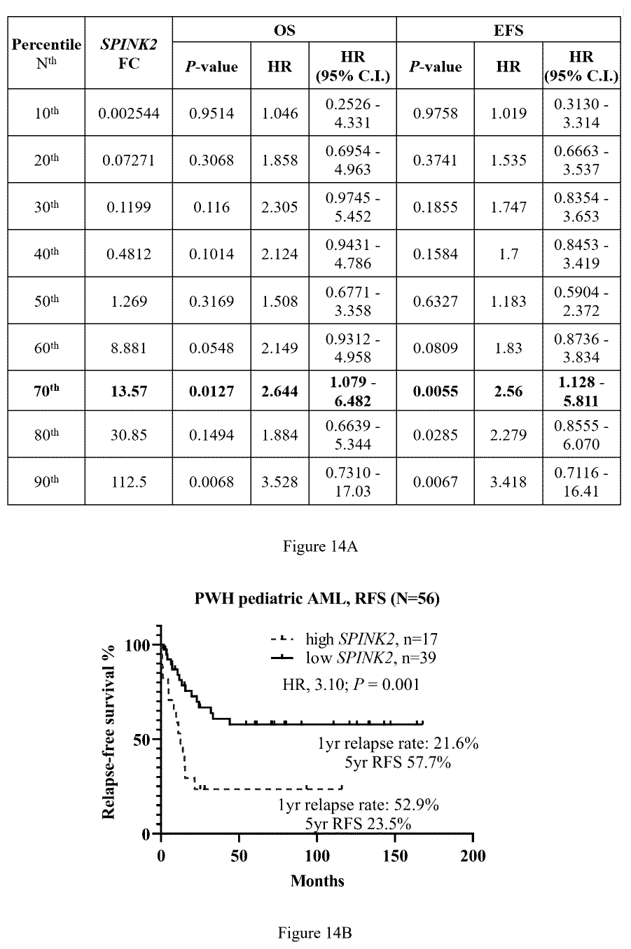

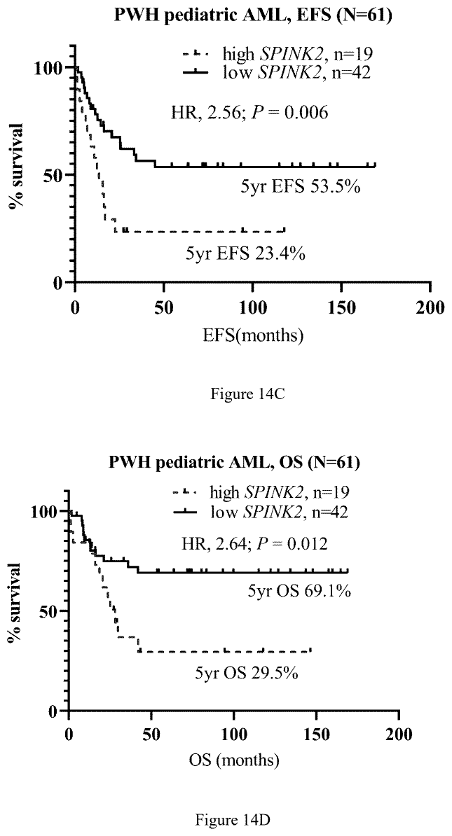

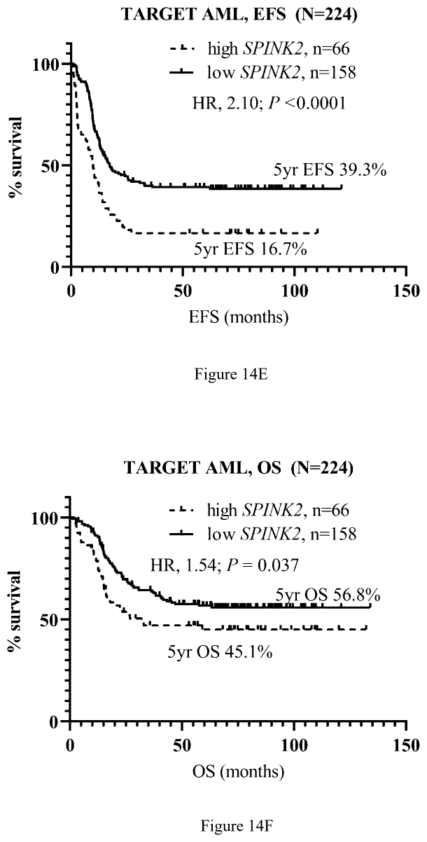

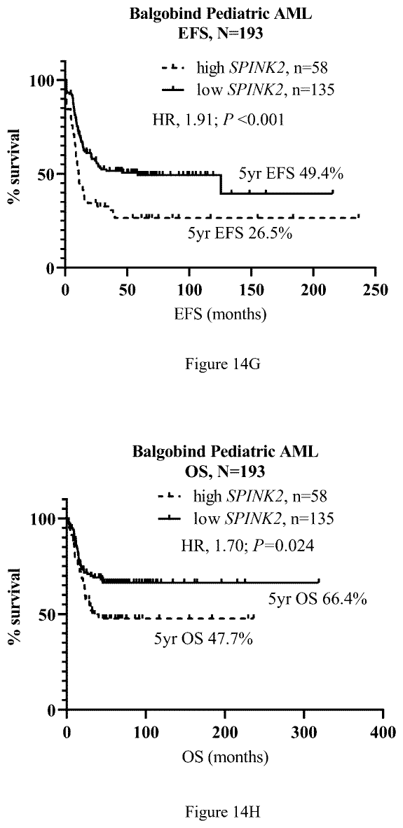

A recent study implicated SPINK2 mRNA overexpression with primary induction failure in a large cohort of pediatric AML patients. The present invention also analyzed SPINK2 mRNA expression by qPCR in the own pediatric cohort of 61 patients and found SPINK2 mRNA overexpression to be associated with intermediate cytogenetic risk, FLT3-ITD mutation, adverse survival and elevated relapse risk (FIGS. 14A-D, Tables S8 & S9). Similar findings are observed in two large independent pediatric AML cohorts (FIGS. 14E-H, Table S10).

As shown in FIG. 14 (A), in order to determine an optimal expression cut-off with strongest prognostic implications, the cohort of 61 patients is first examined by univariate Cox survival analysis comparing OS and EFS using 10% increments of SPINK2 expression fold-change (FC). A cut-off at the 70th percentile demonstrated strongest association with adverse outcome in terms of the log-rank P-value, hazard ratio (HR) and 95% CI of HR. The subsequent Kaplan Meier (KM) survival and clinicopathological analyses are performed by dichotomization of the cohort at this cut-off. (B) KM survival curve for RFS in pediatric AML patients who have achieved CR: Median RFS (high vs low SPINK2)-12.5 months vs unreached. (C) KM survival curve for EFS in pediatric AML patients: Median EFS (high vs. low SPINK2)-13.5 months vs. unreached. (D) KM survival curve for OS in pediatric AML patients: Median OS (high vs. low SPINK2)-28.1 months vs. unreached. (E) KM survival curve for EFS in the TARGET AML cohort: Median EFS (high vs. low SPINK2)-9.9 vs. 17.3 months. (F) KM survival curve for OS in the TARGET AML cohort: Median OS (high vs. low SPINK2)-32.5 months vs. unreached. (G) KM survival curve for EFS in the Balgobind cohort: Median EFS (high vs. low SPINK2)-11.0 vs. 58.4 months. (H) KM survival curve for OS in the Balgobind cohort: Median OS (high vs. low SPINK2)-32.3 months vs. unreached. Survival proportions are compared using the logrank P-value and logrank hazard ratio (HR).

TABLE S8

| | |
|---|---|
| Characteristic | Value |

Age

| | | |
|---|---|---|
| Range (median), yrs. | 0.25-18 | (11) |
| Gender: Male (%) | 37 | (61%) |
| Female (%) | 24 | (39%) |

WBC count, ×10⁹/L

| | | |
|---|---|---|
| Range (median) | 0.7-352 | (15.4) |

Cytogenetic Risk Category

| | | |
|---|---|---|
| Favorable (%) | 20 | (32.8%) |
| Intermediate (%) | 26 | (42.6%) |
| Adverse (%) | 15 | (24.6%) |

Treatment Protocol

| | | |
|---|---|---|
| AML96 | 15 | (24.6%) |
| AML2004 | 30 | (49.2%) |
| AML2012 | 14 | (23.0%) |
| Others | 2 | (3.3%) |

Allo-SCT

| | | |
|---|---|---|
| Yes (%) | 11 | (18.0%) |
| No (%) | 50 | (82.0%) |

Mutations

| | | |
|---|---|---|
| FLT3-ITD | 5 | (8.2%) |
| NPM1 | 2 | (3.3%) |
| CEBPA | 5 | (8.2%) |
| DNMT3A | 2 | (3.3%) |

TABLE S9
Supplementary Table S9. Correlation of clinicopathological
characteristics and therapy response of the pediatric
AML cohort with SPINK2 mRNA expression

| | High | Low | |
|---|---|---|---|
| | SPINK2 | SPINK2 | |
| Factor | N = 19 | N = 42 | P-value^{‡} |
| Age | 10.87 | 11.1 | 0.78 |

Sex

| | | | |
|---|---|---|---|
| male | 52.60% | 64.29% | 0.41 |
| female | 47.40% | 35.71% | |
| WBC (×10⁹/L) | 25.8 | 12.1 | 0.42 |

Cytogenetic risk

| | | | |
|---|---|---|---|
| Favorable | 10.50% | 42.90% | 0.018 |
| Intermediate | 73.70% | 28.60% | 0.002 |
| Adverse | 15.80% | 28.60% | 0.35 |

Mutations

| | | | |
|---|---|---|---|
| NPM1 | 5.26% | 2.44% | 0.54 |
| FLT3-ITD | 21.10% | 2.44% | 0.03 |
| FLT3-PM | 0% | 2.44% | 0.99 |
| NRAS | 15.79% | 17.07% | 0.99 |
| KRAS | 10.53% | 7.32% | 0.65 |
| CEBPA | 5.26% | 2.44% | 0.54 |
| DNMT3A | 5.26% | 2.44% | 0.54 |
| WT1 | 21.05% | 12.20% | 0.45 |
| KIT | 0.00% | 9.76% | 0.3 |
| PTPN11 | 10.53% | 7.32% | 0.65 |

Therapy response

| | | | |
|---|---|---|---|
| CR1 | 63.16% | 69.05% | 0.77 |
| CR | 89.50% | 92.90% | 0.64 |
| 1 yr-relapse rate | 52.90% | 21.60% | 0.001 |
| 2 yr-relapse rate | 76.50% | 33.20% | 0.001 |

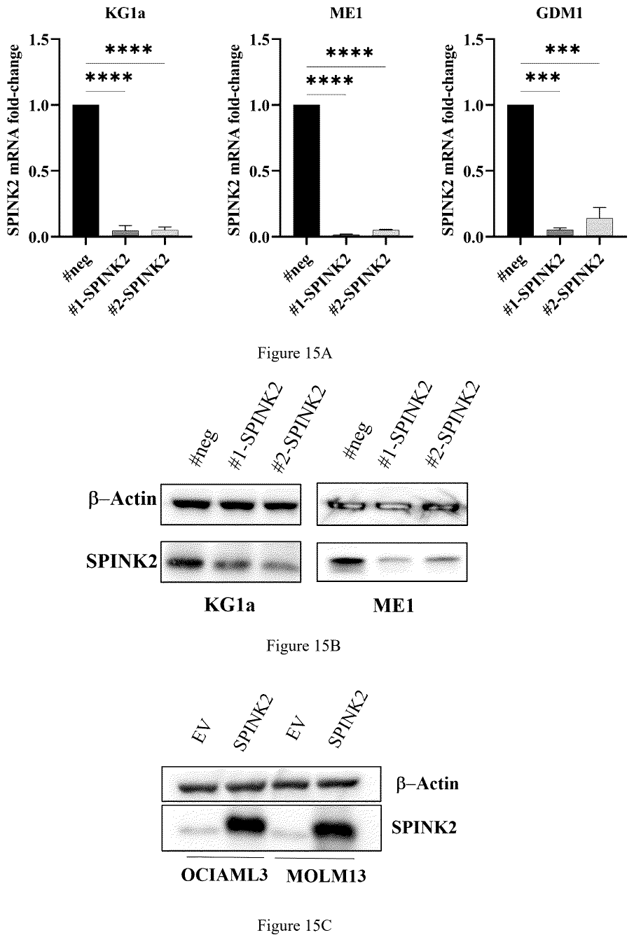

Dichotomization into high and low SPINK2 groups is done by the 70^{th} percentile (Supplementary FIG. 15A)
^{‡}P-value calculate by Fisher's exact test, and significant associations highlighted in bold
Abbreviations: ITD - internal tandem duplication,
dm - double mutation,
CR1 - complete response at 1^{st} induction,
CR- complete response at any time

TABLE S10

| | TARGET-AML (N = 235) | | | Balgobind (N = 193) | | |
|---|---|---|---|---|---|---|
| | High | Low | | High | Low | |
| | SPINK2 | SPINK2 | P | SPINK2 | SPINK2 | P |
| Cytogenetics | n = 70 | n = 165 | value^{‡} | n = 58 | n = 135 | value^{‡} |
| Cytogenetics | | | | | | |
| Normal | 28/65 | 38/158 | 0.006 | 19/52 | 18/121 | 0.002 |
| | (43.1%) | (24.1%) | | (36.5%) | (14.9%) | |
| t (8; 21) | 0/65 | 36/158 | <0.0001 | 0/52 | 27/121 | <0.0001 |
| | (0.0%) | (22.8%) | | (0.0%) | (22.3%) | |
| inv(16) | 6/65 | 24/158 | 0.28 | 4/52 | 22/121 | 0.1 |
| | (9.2%) | (15.2%) | | (7.7%) | (18.2%) | |
| MLL | 9/65 | 29/158 | 0.56 | 14/52 | 24/121 | 0.32 |
| rearrangement | (13.9%) | (18.4%) | | (26.9%) | (19.8%) | |
| Mutations | | | | | | |
| NPM1 | 7/69 | 9/158 | 0.26 | 9/58 | 7/135 | 0.02 |
| | (10.1%) | (5.7%) | | (15.5%) | (5.2%) | |
| FLT3-ITD | 22/70 | 13/165 | <0.0001 | 17/58 | 13/135 | 0.001 |
| | (31.4%) | (7.9%) | | (29.3%) | (9.6%) | |
| Therapy | | | | | | |
| outcome | | | | | | |
| Relapse/ | 53/70 | 98/165 | 0.018 | 41/58 | 57/135 | <0.001 |
| progressive | (75.7%) | (59.4%) | | (70.1%) | (42.2%) | |
| disease | | | | | | |

Supplementary Table S10. Correlation of SPINK2 mRNA overexpression with recurrent AML mutations and cytogenetic aberrations and relapse/progressive disease in 2 independent pediatric AML cohorts: TARGET-AML & Balgobind.
Dichotomization into high and low SPINK2 groups is done by the 70^{th} percentile of SPINK2 mRNA expression.
^{‡}P-value calculated by Fisher's exact test, and significant associations highlighted in bold
Abbreviations:
MLL—Mixed-Lineage Leukemia;
ITD—internal tandem duplication,

Collectively, these findings underline the prognostic importance of SPINK2 expression in AML, and highlight its utilities to refine current prognostic stratification by ELN 2022.

### 1.6.Transcriptome Analysis Reveals a Potential Link Between SPINK2 and Ferroptosis-Related Genes

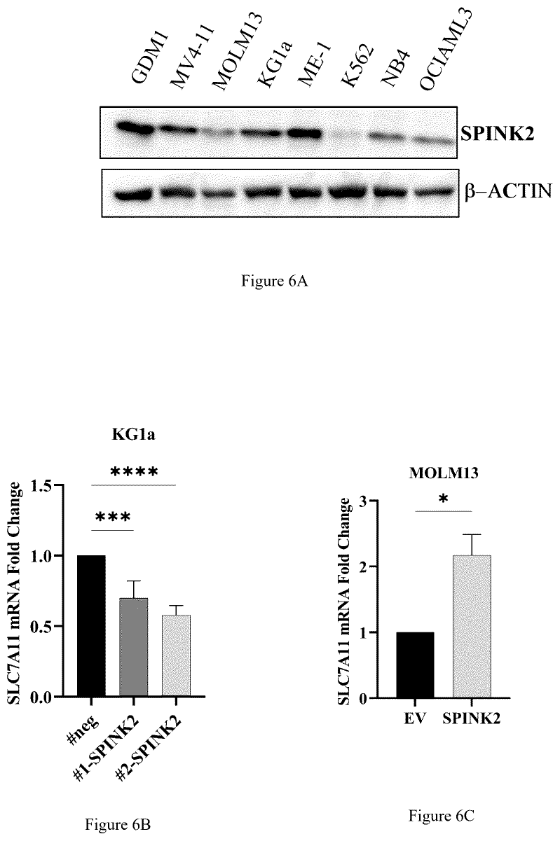

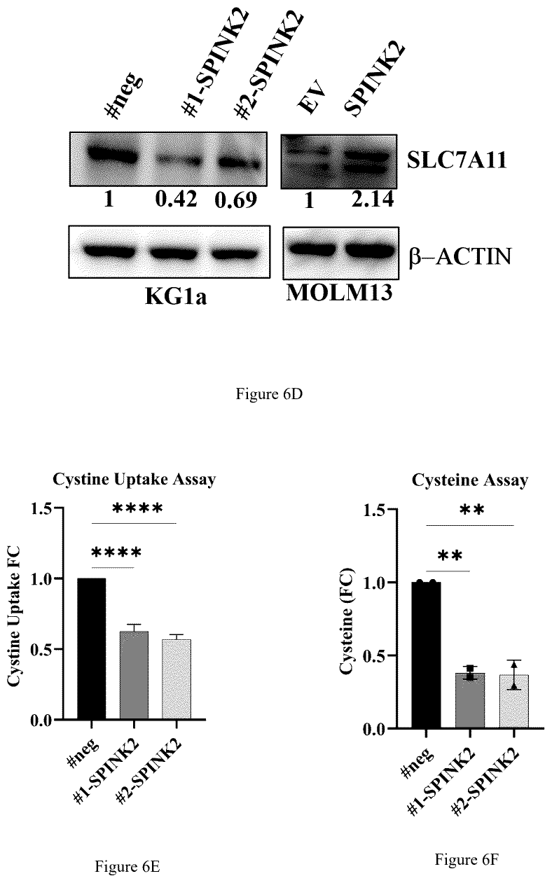

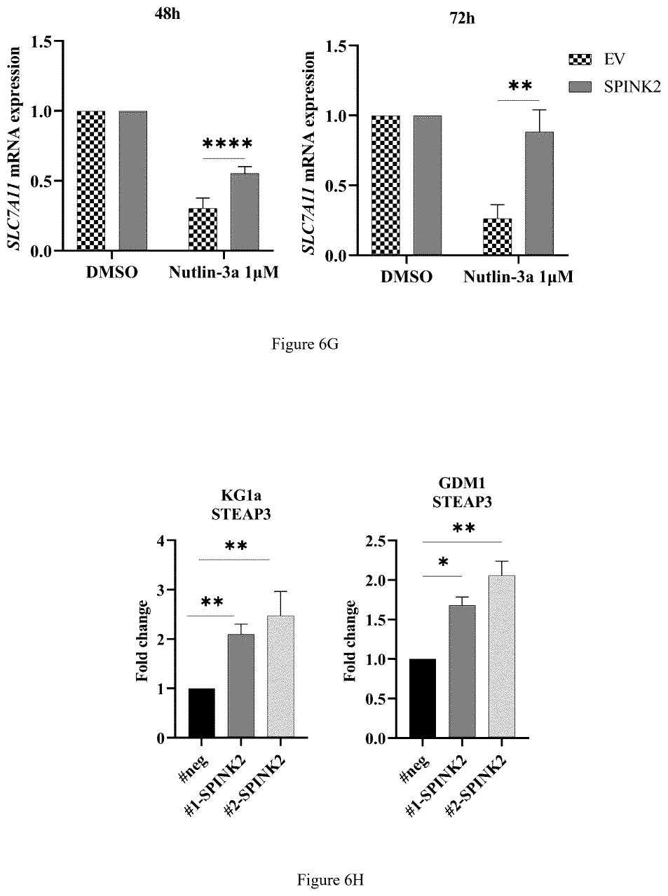

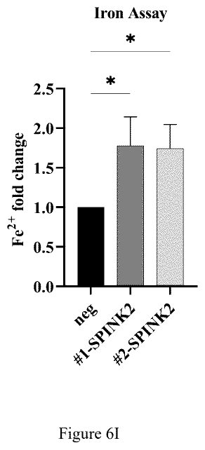

To gain insights into the functional role of SPINK2 in AML, its expression is initially assessed in several AML cell lines by qPCR and Western Blotting showing high expression in CD34+ cells (GDM1, ME1, KG1a) and low/negligible expression in CD34-cells (NB-4, OCIAML3 and MOLM13) (FIG. 6A). In KG1a cells, SPINK2 mRNA is knocked down (KD) with two different SPINK2-targeting siRNAs (#1-siRNA and #2-siRNA) using electroporation. In MOLM13 and OCIAML3 cells, SPINK2 is overexpressed (OE) using GFP-labelled lentiviruses followed by 7-day puromycin selection. Transfection and transduction efficiency data are found in FIGS. 15A-E. Differentially expressed genes (DEGs) between SPINK2-KD and OE cells vs. their respective negative control cells are identified by RNA-sequencing (RNA-seq). SPINK2-KD with siRNA is also performed in SPINK2-high cells ME1 and GDM1 for validation of selected SPINK2 target genes (FIGS. 15A-E).

Since SPINK2 is not a transcription factor, a cut-off of 1.3 (which allowed incorporation of more genes for analysis) is employed to identify commonly deregulated genes/pathways. In two independent experiments of SPINK2-KD in KG1a cells, 76 genes are commonly downregulated, while 99 genes are commonly upregulated by both siRNAs. In MOLM13 and OCIAML3 cells, 31 genes are commonly upregulated, while 68 genes are commonly downregulated upon SPINK2 OE. Gene Set Enrichment Analysis (GSEA) is performed using Hallmark and Gene Ontology (biological processes) datasets of the Molecular Signatures Database (MSigDb). Among the top 10 enriched pathways in each dataset, the following pathways are common to both KD and OE cells: “Interferon Gamma Response”, “Apoptosis” and “P53 pathway” (Tables S11 & S12).

TABLE S11
Supplementary Table S11. Gene Set Enrichment Analysis (GSEA)
using the Molecular Signatures Database (MSigDB) of the
BROAD Institute in KG1a cells with SPINK2-knockdown.

| | | |
|---|---|---|
| GSEA ANALYSIS (MSigDB) in | | FDR |
| KG1a cells | P-value | q-value |

Genes downregulated >1.3-fold
Hallmark datasets

| | | |
|---|---|---|
| INTERFERON_GAMMA_RESPONSE | <0.0001 | <0.001 |
| INTERFERON_ALPHA_RESPONSE | <0.0001 | <0.001 |
| HEME_METABOLISM | <0.0001 | <0.001 |
| FATTY_ACID_METABOLISM | <0.001 | <0.01 |
| APOPTOSIS | <0.001 | <0.01 |
| APICAL_JUNCTION | <0.001 | <0.01 |
| ESTROGEN_RESPONSE_LATE | <0.001 | <0.01 |
| COAGULATION | <0.01 | <0.05 |
| REACTIVE_OXYGEN_SPE- | <0.01 | <0.05 |
| CIES_PATHWAY | | |
| IL2_STAT5_SIGNALING | <0.01 | <0.05 |

Gene ontology (biological processes)

| | | |
|---|---|---|
| CELL_ACTIVATION | <0.0001 | <0.0001 |
| REGULATION_OF_IMMUNE_SYS- | <0.0001 | <0.0001 |
| TEM_PROCESS | | |
| CELL_CELL_ADHESION | <0.0001 | <0.0001 |
| BIOLOGICAL_ADHESION | <0.0001 | <0.0001 |
| EXOCYTOSIS | <0.0001 | <0.0001 |
| DEFENSE_RESPONSE | <0.0001 | <0.0001 |
| RESPONSE_TO_BIOTIC_STIMULUS | <0.0001 | <0.0001 |
| POSITIVE_REGULATION_OF_IM- | <0.0001 | <0.0001 |
| MUNE_SYSTEM_PROCESS | | |
| ORGANIC_ACID_META- | <0.0001 | <0.0001 |
| BOLIC_PROCESS | | |
| CELL_ACTIVATION_IN- | <0.0001 | <0.0001 |
| VOLVED_IN_IMMUNE_RESPONSE | | |

Genes upregulated >1.3-fold
Hallmark datasets

| | | |
|---|---|---|
| P53_PATHWAY | <0.0001 | <0.0001 |
| APOPTOSIS | <0.001 | <0.05 |
| HEME_METABOLISM | <0.01 | <0.05 |

Gene ontology (biological processes)

| | | |
|---|---|---|
| APOPTOTIC_PROCESS | <0.0001 | <0.001 |
| LOCOMOTION | <0.0001 | <0.001 |
| ADIPOSE_TISSUE_DEVELOPMENT | <0.0001 | <0.01 |
| HOMEOSTATIC_PROCESS | <0.0001 | <0.01 |
| REGULATION_OF_ORGAN- | <0.0001 | <0.01 |
| ELLE_ORGANIZATION | | |
| REGULATION_OF_CATA- | <0.0001 | <0.01 |
| BOLIC_PROCESS | | |
| DEFENSE_RESPONSE | <0.0001 | <0.01 |
| CELL_MIGRATION | <0.0001 | <0.01 |
| TRANSMEMBRANE_TRANSPORT | <0.0001 | <0.01 |
| NEGATIVE_REGULATION_OF_PRO- | <0.0001 | <0.01 |
| TEIN_MODIFICATION_PROCESS | | |

Top 10 enriched pathways are shown for Hallmark and Gene Ontology (Biological processes) datasets.

TABLE S12
Supplementary Table S12. Gene Set Enrichment Analysis
(GSEA) using the Molecular Signatures Database
(MSigDB) of the BROAD Institute in MOLM13 & OCIAML3
cells with SPINK2-overexpression

| | | |
|---|---|---|
| GSEA ANALYSIS (MSigDB) in | | FDR |
| MOLM13 & OCIAML3 cells | P-value | q-value |

Genes upregulated >1.3-fold
Hallmark datasets

| | | |
|---|---|---|
| MTORC1_SIGNALING | <0.0001 | <0.0001 |
| UNFOLDED_PROTEIN_RESPONSE | <0.0001 | <0.0001 |
| INTERFERON_GAMMA_RESPONSE | <0.0001 | <0.001 |

Gene ontology (biological processes)

| | | |
|---|---|---|
| CELLULAR_AMINO_ACID_META- | <0.0001 | <0.0001 |
| BOLIC_PROCESS | | |
| ORGANIC_ACID_META- | <0.0001 | <0.0001 |
| BOLIC_PROCESS | | |
| ALPHA_AMINO_ACID_META- | <0.0001 | <0.0001 |
| BOLIC_PROCESS | | |
| CELLULAR_AMINO_ACID_BIO- | <0.0001 | <0.0001 |
| SYNTHETIC_PROCESS | | |
| SMALL_MOLECULE_META- | <0.0001 | <0.0001 |
| BOLIC_PROCESS | | |
| DICARBOXYLIC_ACID_META- | <0.0001 | <0.0001 |
| BOLIC_PROCESS | | |
| CELLULAR_AMIDE_META- | <0.0001 | <0.0001 |
| BOLIC_PROCESS | | |
| SERINE_FAMILY_AMI- | <0.0001 | <0.0001 |
| NO_ACID_METABOLIC_PROCESS | | |
| ORGANONITROGEN_COM- | <0.0001 | <0.0001 |
| POUND_BIOSYNTHETIC_PROCESS | | |
| CELLULAR_MODIFIED_AMI- | <0.0001 | <0.001 |
| NO_ACID_METABOLIC_PROCESS | | |

Genes downregulated >1.3-fold
Hallmark datasets

| | | |
|---|---|---|
| CHOLESTEROL_HOMEOSTASIS | <0.0001 | <0.0001 |
| HYPOXIA | <0.0001 | <0.0001 |
| IL2_STAT5_SIGNALING | <0.0001 | <0.0001 |
| APOPTOSIS | <0.0001 | <0.0001 |
| MTORC1_SIGNALING | <0.0001 | <0.0001 |
| IL6_JAK_STAT3_SIGNALING | <0.0001 | <0.001 |
| APICAL_JUNCTION | <0.0001 | <0.001 |
| P53_PATHWAY | <0.0001 | <0.001 |
| ANDROGEN_RESPONSE | <0.0001 | <0.001 |
| UV_RESPONSE_UP | <0.001 | <0.001 |

Gene ontology (biological processes)

| | | |
|---|---|---|
| LIPID_METABOLIC_PROCESS | <0.0001 | <0.0001 |
| LIPID_BIOSYNTHETIC_PROCESS | <0.0001 | <0.0001 |
| STEROL_BIOSYNTHETIC_PROCESS | <0.0001 | <0.0001 |
| SMALL_MOLECULE_META- | <0.0001 | <0.0001 |
| BOLIC_PROCESS | | |
| SECONDARY_ALCOHOL_META- | <0.0001 | <0.0001 |
| BOLIC_PROCESS | | |
| STEROL_METABOLIC_PROCESS | <0.0001 | <0.0001 |
| ALCOHOL_METABOLIC_PROCESS | <0.0001 | <0.0001 |
| STEROID_BIOSYNTHETIC_PROCESS | <0.0001 | <0.0001 |
| CELL_ADHESION | <0.0001 | <0.0001 |
| ORGANIC_HYDROXY_COM- | <0.0001 | <0.0001 |
| POUND_METABOLIC_PROCESS | | |

Top 10 enriched pathways are shown for Hallmark and Gene Ontology (Biological processes) datasets.

Two genes are commonly upregulated in SPINK2-OE cells and downregulated in SPINK2-KD cells: SLC7A11 and ASNS. SLC7A11 is a specific cystine/glutamate antiporter and a master regulator of ferroptosis. Furthermore, studies have shown that SLC7A11 overexpression associates with poor prognosis in AML, and that ferroptosis induction represents a novel treatment strategy. Therefore, the present invention investigated the relationship of SPINK2 and SLC7A11 more carefully.

qPCR and Western Blots confirmed the modulation of SLC7A11 expression upon SPINK2-KD and OE in KG1a and MOLM13 cells (FIG. 6B-D). SPINK2-KD in KG1a cells resulted in decreased cystine uptake and intracellular cysteine levels, which are functional consequences of SLC7A11 downregulation (FIGS. 6E, F).

Previously, p53 transcriptionally represses SLC7A11 expression, thereby playing an important pro-ferroptotic role. The data has shown that p53 pathway genes are inversely affected upon SPINK2 modulation (Tables S11 & S12). Based on the hypothesis that SPINK2 overexpression in MOLM13 cells might counteract the p53-mediated repression of SLC7A11, MOLM13-EV and MOLM13-SPINK2 cells are treated with the p53 activator, Nutlin-3a (1 μM), for 48 hours and 72 hours. Indeed, SLC7A11 mRNA expression is reduced in MOLM13-EV cells to a significantly greater extent than in MOLM13-SPINK2 cells (FIG. 6G). Of note, the effects of Nutlin-3a could not be tested in KG1a cells, since they have a TP53 mutation which renders them insensitive to Nutlin-3a activity.

Another notable finding is the consistent overexpression of STEAP3 in KG1a and GDM1 cells with SPINK2-KD (FIG. 6H). STEAP3 is transcriptionally activated by p53, and acts as a ferrireductase (which reduces intracellular ferric (Fe³⁺) to ferrous (Fe²⁺) iron) to increase the intracellular labile iron pool. Increased intracellular Fe²⁺ is a hallmark of ferroptosis. Functionally, intracellular Fe²⁺ levels are significantly increased upon SPINK2-KD in KG1a cells (FIG. 6I).

Collectively, the present invention reveals that SPINK2 serves to counteract p53-mediated ferroptosis induction by modulating the expression of its downstream targets, SLC7A11 and STEAP3.

### 1.7.Genetic and Pharmacologic Modulation of SPINK2 Expression Influences Sensitivity to Erastin, a Ferroptosis Inducer

These intracellular changes due to SPINK2-KD might render the cells more susceptible to ferroptosis induction.

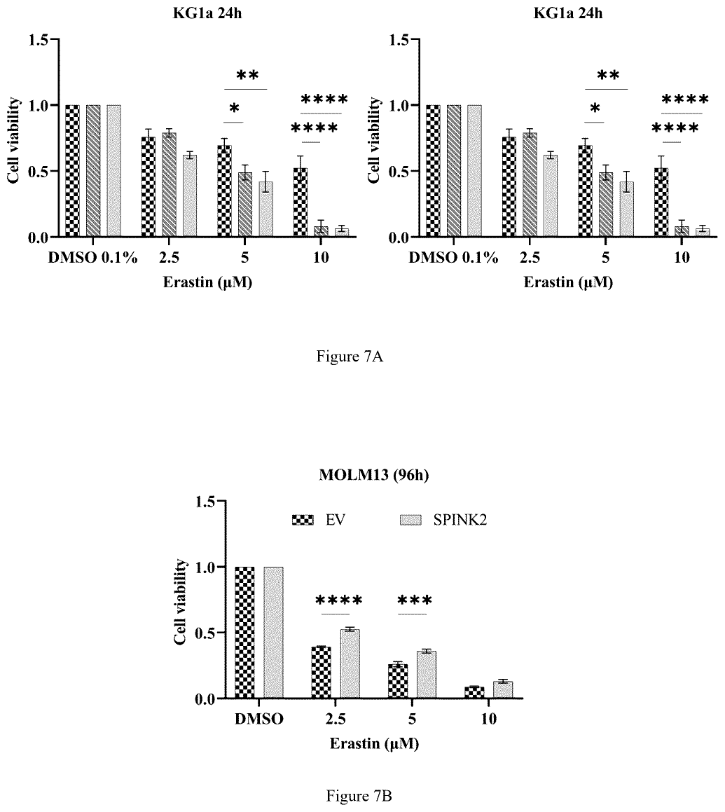

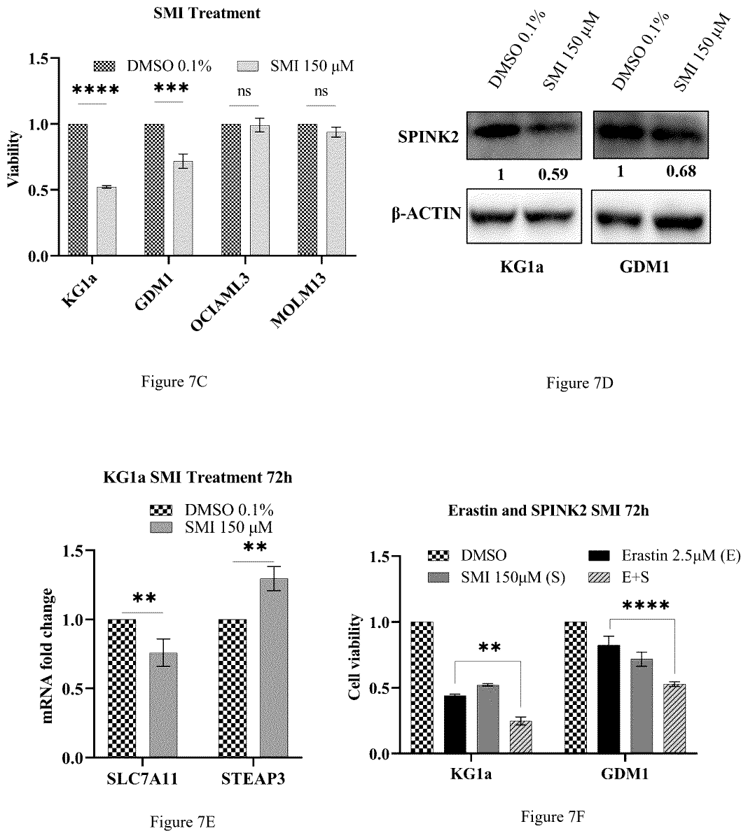

The effects of SPINK2 modulation upon ferroptosis are examined employing erastin, a potent ferroptosis inducer. 48 hours after SPINK2-KD, KG1a cells are treated with a range of erastin doses (2.5 μM-10 μM) for 24 hours to 48 hours. Cell viability is significantly reduced in the SPINK2-KD cells vs. negative control upon erastin treatment (FIG. 7A). Next, MOLM13-SPINK2 and MOLM13-EV cells are similarly treated with erastin for 48 hours to 96 hours. MOLM13-SPINK2 cells are significantly more resistant to cell death by erastin at 96th hour (FIG. 7B).

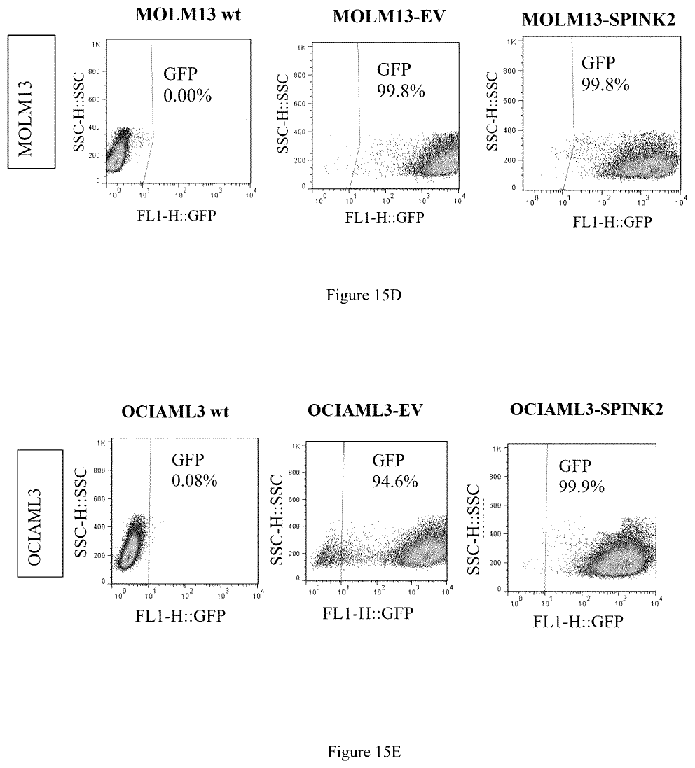

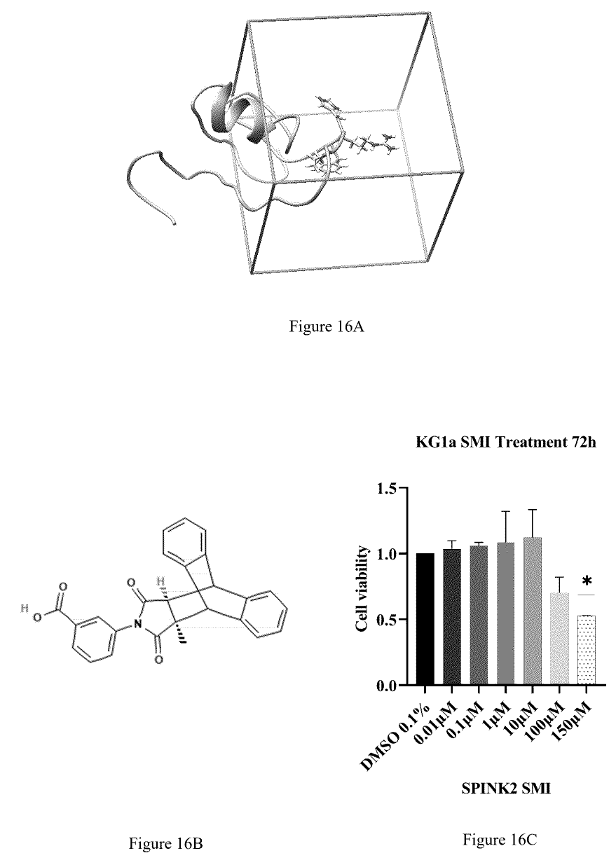

To identify potential SPINK2-SMIs, Structure-based Virtual Screening (SBVS) is initially employed for in silico screening of a small-molecule library comprising 1.5 million compounds to identify bioactive molecules that bind to the targeting domain of SPINK2 (FIG. 16A). Among the top 1000 compounds with higher affinity to SPINK2 based upon their idock scores, only one of these compounds (C₂₆H₁₉NO₄, PubChem CID: 1102833) (FIG. 16B) is soluble in DMSO, and is therefore chosen for further analysis. The only three compounds are insoluble in DMSO and other available solvents, such as water, ethanol and dimethylformamide (DMF).

The SMI is initially tested with increasing doses for its effect upon cell viability in KG1a cells. At 72^{th} hour, 150 μM treatment reduced cell viability by approximately 50% (FIG. 16C). This dose is then tested on GDM1, MOLM13 and OCIAML3 cells. SMI treatment (150 μM for 72 hours) significantly decreased cell viability of SPINK2^{high} cells (KG1a, GDM1) but not SPINK2^{low} cells (OCIAML3, MOLM13) (FIG. 7C). SPINK2 protein expression is also decreased in KG1a and GDM1 cells by SMI treatment (FIG. 7D). Additionally, SMI treatment of KG1a cells resulted in alteration of SPINK2 target gene mRNA expression, namely downregulation of SLC7A11 and upregulation of STEAP3 (FIG. 7E), which is consistent with effects observed by genetic SPINK2 inhibition with siRNAs.

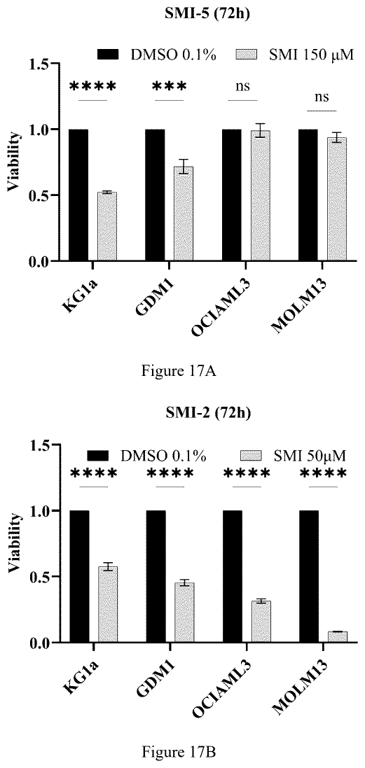

Further examples of the screened SMIs are illustrated in FIGS. 17A-B wherein FIG. 17A shows cytotoxicity data for the SMI (here designated as ‘SMI-5’) and FIG. 17B shows cytotoxicity data for another potential SMI (here designated as ‘SMI-2’). Cells are treated with the SMIs and control (DMSO) for 72 hours and their viability is detected using the Cell Titer Glo® assay according to the manufacturer's instructions. Statistical analysis is performed using one-way ANOVA with Tukey's multiple comparisons test with Mean+SD of at least 2 independent experiments. The findings demonstrate greater toxicity of the SMI (SMI-5) against SPINK2^{high} cells (KG1a and GDM1) vs. SPINK2^{low} cells (OCIAML3 and MOLM13) when compared with another potential SMI (SMI-2).

The effects of pharmacologic SPINK2 inhibition with the SMI on erastin are also examined. Wildtype KG1a and GDM1 cells are treated with a combination of erastin (2.5M) and/or SPINK2-SMI (150 μM) for 72 hours. Combined erastin/SMI treatment significantly reduced cell viability compared to erastin alone (FIG. 7F). Collectively, these findings further support the SPINK2 in the regulation of ferroptosis.

1.8.SPINK2 Modulation Affects Expression of Immune-Response Related Genes in LSC-Like SPINK 2^{high} Cells

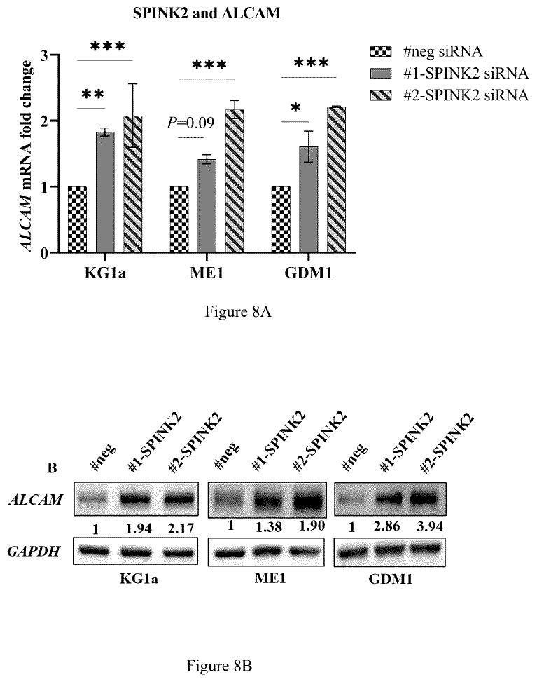

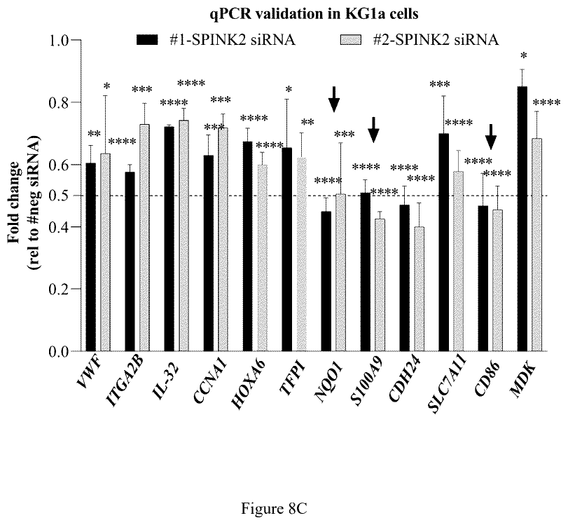

Avoiding destruction by the immune system is one of the several hallmarks of cancer cells. Immune evasion is indeed a prominent characteristic of AML blasts and LSCs. The analysis uncovered a potential link between SPINK2 and immune response regulation. Among the DEGs in SPINK2-KD KG1a cells, the expression of several immune response related genes is strongly altered (>2-fold). Among upregulated genes is Activated Leukocyte Cell Adhesion Molecule (ALCAM), a potent T-cell activator. Interestingly, ALCAM expression is consistently increased in the LSC-like KG1a, ME1 and GDM1 cells with SPINK2-KD (FIGS. 8A, B). Additionally, T-cell activity inhibitory genes (CD86, S100A9, NQO1) are downregulated in KG1a cells. This is validated by qPCR in three independent knockdown experiments (FIG. 8C, with arrow indicators). GSEA analysis has indeed shown that pathways involved in immune system regulation are affected in KG1a cells (Table S11). Collectively, these findings suggest that SPINK2 in AML-LSCs might contribute to immune evasion by suppression of T-cell activity.

### Supplementary Methods and Information

### Identification of SPINK2 Overexpression in AML and Functionally Defined LSC Fractions

The Oncomine database is used to initially compare microarray gene expression data between AML samples (N=831) and normal bone marrow (NBM) samples (N=141) in four independent datasets (GSE7186, GSE13164, GSE13159, GSE995) in generating a list of differentially expressed genes. The top-50 genes by median-ranked analysis are further selected. Out of these 50 genes, only genes that are (i) not well characterized in AML, and (ii) part of a recently generated LSC gene signature are further selected. Four genes are selected by these criteria: SHANK3, GPSM1, FSCN1 and SPINK2. Median expression of the four genes is then compared between sorted CD34+ AML cells (n=46) and sorted CD34+ NBM cells (n=31) in the GSE30029 dataset. Of the four genes, SPINK2 has significantly highest fold-change (SPINK2: 2.34, p=0.0065; FSCN1: 1.53, p=0.004; GPSM1: 1.37, p-0.086; SHANK3: 1.29, p=0.19). Furthermore, median expression of these genes is also compared between functionally defined LSC-enriched (LSC+, n=25) and LSC-depleted (LSC-, n=29) populations in the dataset (GSE30377). SPINK2 and FSCN1 are significantly upregulated in LSC+vs. LSC-populations (SPINK2: 1.653 vs.-0.2122, P=0.032; FSCN1: 0.2649 vs.-0.3189, P=0.034), whereas no data are available for the other two genes (SHANK3, GPSM1). In one of the datasets, SPINK2 is increased approximately 4-fold in the functionally defined LSC fraction vs non-LSC fraction, while FSCN1 is increased around 2.5-fold (Data obtained from original study, extended data table 1 “List of 104 DE LSC genes”). Based upon these initial observations, SPINK2 is chosen for further analysis. From the initial Oncomine analysis, SPINK2 expression is significantly increased more than 2-fold in AML vs. NBM in all 4 datasets. Further Oncomine analyses of relative SPINK2 gene expression among 3,248 leukaemia patients (AML, CML, ALL, CLL) demonstrated relatively high SPINK2 expression specifically in AML patients.

### In-House Adult AML Patient Dataset and Exclusion Criteria for Survival Analysis

A total of 172 non-M3 adult AML patients treated at the Prince of Wales Hospital (PWH) in Hong Kong are recruited into the study. Archival formalin-fixed paraffin-embedded diagnostic bone marrow trephine biopsies or clots are analysed for SPINK2 protein expression by immunohistochemistry (IHC) using the fully automated Ventana BenchMark ULTRA. 35 patients are excluded from the survival and treatment-response analyses because of the following reasons: (i) secondary or therapy-related AML, or AML with myelodysplasia-related changes (n=10); (ii) not receiving standard induction therapy with the Daunorubicin-Cytarabine (DA) 3+7 backbone (n=14); (iii) loss of clinical follow up (n=5); or (iv) death within days of diagnosis or induction (n=6). Thus, for more accurate and non-biased survival and treatment-response analyses, a relatively homogeneous cohort of 137 de novo AML receiving standard DA 3+7 backbone regimens at induction is studied. 41 patients received SCT, of which only 37 are included in the survival and treatment response analysis based upon the exclusion criteria mentioned above. To examine the association of SPINK2 status and SCT outcome, an additional 77 SCT recipients with de novo AML and receiving DA 3+7 induction therapy backbone are recruited from partner hospitals to generate a combined SCT cohort (N=114). Of these, 82 (71.9%) patients received SCT at CR1, while the remainder received SCT as salvage-either in relapse or primary refractory status. Data collection for clinical information is ended in March 2021.

### Definition of Clinical End-Points

Overall survival (OS) is defined as the time from date of diagnosis until date of last follow-up or death by any cause. Event-free survival (EFS) is defined as time elapsed from date of diagnosis until date of first leukemic event (non-response to therapy, relapse or death) or last follow-up. Relapse-free survival (RFS) is defined as time elapsed from date of achievement of complete remission (CR) until date of relapse or death (from any cause) or last follow-up. For the transplant analysis, post-SCT OS is defined as the time elapsed from receipt of SCT until death from any cause or last clinical follow-up. CR is defined according to standard criteria.

### Public Datasets Used for Validation of Clinical Findings in Adult and Pediatric AML

### TCGA-LAML (N=200)

RNA Sequencing data is available for 173 out of 200 patients included into The Cancer Genome Atlas (TCGA) adult AML study. SPINK2 RPKM expression values are downloaded for each patient from cBioPortal with detailed clinical and mutational information for 200 patients. A value of 1 is added to each RPKM value before log 2-transformation is performed. Patients are dichotomized into higher and lower SPINK2 expression groups by the median to analyse the correlation of SPINK2 expression with cytogenetic and mutational status. Out of the 173 patients, 58 patients are excluded from the survival analysis because they either are of FAB M3 subtype (N=16); received induction with therapeutics not involving the standard DA 7+3 regimen backbone (N=36); has OS<1 month (N=4); or has incomplete data (N=2). This left a more homogeneously treated subgroup of 115 patients. Of note, only OS data is available for analysis. For survival analysis, the heterogeneous cohort (N=115) and subgroups are dichotomized at the median into high and low SPINK2 groups. For the pairwise multivariate Cox analysis comparing LSC gene expression signatures and SPINK2 expression, three previously published LSC gene expression signatures are used. The scores of each patient sample are calculated using the gene signatures as described in the respective publications.

### OHSU BEAT-AML, N=672

RNA-Sequencing data for SPINK2 is available for 405/672 patients included into the BEAT AML study. Of these, patients not having a diagnosis of AML (N=13) are excluded, leaving 392 patients with complete mutational data for analysis. SPINK2 RPKM expression values are downloaded for each patient from cBioPortal, including mutational, cytogenetic and clinical information for each patient. For analysis of SPINK2 and chemotherapy response, 180 patients are analyzed since they (i) are without a diagnosis of AML with myelodysplasia-related changes or therapy-related AML; (ii) are treated on standard induction regimens involving cytarabine and anthracycline backbones; and (iii) has available data on treatment response.

### Verhaak (GSE6891, N=537)

This dataset comprises 537 adult de novo AML patients≤60 years of age treated according to the protocols of the Dutch-Belgian Haematology-Oncology Cooperative Group. Log-transformed microarray gene expression data and other relevant clinical data available for 458 patients are downloaded from NCBI GEO database. After excluding 17 patients with MDS and 24 patients with FAB M3, 417 patients are included for clinicopathological analysis. Patients are dichotomized into high and low SPINK2 groups by the median.

### Pediatric AML (GSE17855, N=237)

Microarray gene expression data of this cohort are downloaded from NCBI GEO with and clinical data of the patients. Only 193 out of 237 patients are included into the survival and treatment-response analysis after exclusion of patients having no survival data (N=16), patients with OS less than 1 month (N=14), and patients with t (15;17) AML (N=14).

TARGET-AML (pediatric), N=235

Freely accessible RNA Sequencing data as well as clinical data available for 235 non-FAB M3 patients of this cohort are downloaded. 224 patients are included into the survival and treatment-response analysis after exclusion of patients above age 18yrs (N=10) and patients with OS<1 month (N=1).

### Materials and Methods for Classifying SPINK2

### 2.1.Antibodies and Drugs

The following primary antibodies are selected: SPINK2 (#HPA026813), SLC7A11 (#12691S), ALCAM (#ab109215), β-Actin (#ab8266,) and GAPDH (#ab9485). The following drugs are used: Dimethyl sulfoxide (DMSO, #D4540), Nutlin-3a (#S8059), erastin (#5499), Puromycin (#A1113802) and C₂₆H₁₉NO₄ (#OSSK_987997), which are used at the concentrations: DMSO: 0.1% Nutlin-3a: 1 μM Erastin: concentration range (2.5-10 μM) Puromycin: 1 μg/ml C₂₆H₁₉NO₄: 150 μM.

### 2.2.Immunohistochemistry (IHC)

IHC for SPINK2 expression is performed on the fully automated Ventana Benchmark ULTRA platform. Specimens are sectioned at a thickness of 4 μm, stained on positively charged glass slides and stored at room temperature until further use. Initially, the slides are warmed at 70° C. for 10-15 min. Deparaffinization, rehydration, and antigen retrieval are performed on the Ventana automated slide stainer using CC1 antigen retrieval solution at 100° C. for 64 min. Incubation with the rabbit polyclonal primary SPINK2 antibody HPA026813 (Sigma-Aldrich) is performed at a dilution of 1:100 for 32 min at 36° C. The OptiView DAB (3,3′-Diaminobenzidine) IHC Detection Kit v5 is then used for visualization, involving post-primary peroxidase blocking for 4 min, and incubation with Linker and Multimer solutions for 12 min each. Slides are then incubated with hydrogen peroxide and DAB for 8 min, followed by copper enhancement for 4 min. Next, counterstaining is performed with Mayer's Haematoxylin for 1-2 mins, followed by bluing agent for 1 min, followed by standard manual dehydration with ethanol and xylene. Slides are coverslipped and warmed for 10 min prior to microscopic analysis. Normal testicular tissue served as a positive control (with buffer and primary antibody) and negative control (with buffer, without primary antibody). Slide images are captured using Nikon Ni-u Light Microscope.

2.3.SPINK2 IHC score calculation and prognostic cut-off determination

SPINK2 IHC expression is assessed independently blinded to each other and to the clinical data of the patients. Quantification of SPINK2 expression is achieved through a composite SPINK2 IHC score employing the percentage of stained blasts (P) and the intensity of the staining (I). ‘P’ values are as follows: <20%=1, 20-50%=2, 50-75%=3, >75%=4. ‘I’ values are as follows: negative-0, mild-1, moderate-2, strong-3, very strong-4. Each patient received a unique score calculated as ‘P×I’. The minimum score is 0, the maximum score is 16. The average of the pathologists' scores is assigned as the final score for each patient.

Further and/or alternatively, in order to determine an optimal expression cut-off with strongest prognostic implications, the cohort of 137 patients is initially divided into 4 quartiles (q1, q2, q3 & q4) based upon SPINK2 score distribution (q1: score 0, q2: score 1-3, q3: score 4-7, q4: score 8-16). Kaplan-Meier univariate survival analyses for OS and EFS showed that dichotomizing patients by the median score of ‘3’ has the strongest association with adverse outcome in terms of the log-rank P-value and hazard ratio (HR) when each quartile is compared with the others.

2.4.RNA Extraction, Quantitative Polymerase-Chain Reaction (qPCR)

Table S3 below tabulates the correlation of SPINK2 mRNA overexpression with recurrent AML mutations and cytogenetic aberrations in three independent adult AML cohorts.

TABLE S3

| | TCGA-LAML (N = 173) | | | OHSU-BEAT-AML (N = 392) | | | Verhaak (N = 417) | | |
|---|---|---|---|---|---|---|---|---|---|
| | High | Low | | High | Low | | High | Low | |
| Mutations & | SPINK2 | SPINK2 | P | SPINK2 | SPINK2 | P | SPINK2 | SPINK2 | P |
| Cytogenetics | n = 87 | n = 86 | value^{‡} | n = 196 | n = 196 | value^{‡} | n = 208 | n = 209 | value^{‡} |
| Mutations | | | | | | | | | |
| NPM1 | 31(35.63%) | 17(19.77%) | 0.027 | 62(31.6%) | 32(16.3%) | 0.0006 | 89(42.8%) | 48(23.0%) | <0.0001 |
| FLT3-ITD | 25(28.74%) | 12(13.95%) | 0.025 | 59(30.1%) | 28(14.3%) | 0.0002 | 81(38.9%) | 36(17.2%) | <0.0001 |
| CEBPAdm | 1(1.15%) | 4(4.65%) | 0.211 | N/A | N/A | N/A | 6(2.9%) | 17(8.1%) | 0.029 |
| DNMT3A | 28(32.18%) | 15(17.44%) | 0.034 | 54(27.6%) | 28(14.3%) | 0.002 | N/A | N/A | N/A |
| Cytogenetics^{#} | | | | | | | | | |
| Normal | 47(55.3%) | 33(38.8%) | 0.045 | 93(51.7%) | 62(35.6%) | 0.003 | 104(55.6%) | 75(38.9%) | 0.001 |
| t (8; 21) | 0(0.00%) | 7(8.24%) | 0.014 | 0(0.00%) | 8(4.6%) | 0.003 | 0(0.0%) | 35(18.1%) | <0.0001 |
| inv (16) | 3(3.61%) | 7(8.24%) | 0.329 | 8(4.4%) | 17(9.8%) | 0.062 | 10(5.4%) | 23(11.9%) | 0.028 |
| Cytogenetic risk^{$} | | | | | | | | | |
| Favorable | 8(9.4%) | 24(28.2%) | 0.003 | N/A | N/A | N/A | 11(5.4%) | 63(31.0%) | <0.0001 |
| Intermediate | 58(68.2%) | 43(50.6%) | 0.028 | N/A | N/A | N/A | 146(71.6%) | 101(49.8%) | <0.0001 |
| Adverse | 19(22.4%) | 18(21.2%) | >0.99 | N/A | N/A | N/A | 47(23.0%) | 39(19.2%) | 0.396 |
| ELN 2022 risk* | | | | | | | | | |
| Favorable | 23(26.4%) | 41/85(48.2%) | 0.004 | N/A | N/A | N/A | N/A | N/A | N/A |
| Intermediate | 33(37.9%) | 15/85(17.5%) | 0.004 | N/A | N/A | N/A | N/A | N/A | N/A |
| Adverse | 31(35.6%) | 29/85(34.1%) | 0.87 | N/A | N/A | N/A | N/A | N/A | N/A |

Supplementary table S3.
Dichotimization into high and low SPINK2 is done by median SPINK2 mRNA expression
^{‡}P-value calculate by Fisher's exact test
^{#}Cytogenetic status is available for only 170 patients in the TCGA-LAML cohort, 354 patients in the OHSU-BEAT-AML cohort and 380 patients in the Verhaak cohort.
^{$}Cytogenetic risk is defined by the authors of the respective studies, and is available for only 170 patients in the TCGA-LAML cohort and 407 patients in the Verhaak cohort.
*ELN 2022 risk could only be determined for 172 patients of the TCGA-LAML cohort.
Abbreviations:
ITD—internal tandem duplication,
dm—double mutation,
N/A—data not available

Total RNA is extracted using the QIAamp RNA Blood Mini Kit. cDNA is synthesized using the Superscript III First-Strand Synthesis System according to the manufacturer's instructions. qPCR is performed using the real-time PCR system. In a further and/or alternative embodiment, the following conditions are employed: Hold (50° C., 2 min)-Hold (95° C.-10 mins)-40 cycles (95° C.,15s-60° C.,1 min). The following TaqMan® Gene Expression Assay is used for SPINK2: Hs01598293_m1. Each sample is measured in triplicate and gene expression is analysed by the 2-AACt method, GAPDH is used as housekeeping gene for normalization. The relative fold-change of SPINK2 in clinical samples is compared to the expression in sorted CD34+ cord-blood cells. RNA is available for 128 patients, and SPINK2 mRNA levels are assessed by qPCR for correlation analysis with IHC scores in these patients.

### 2.5.Targeted Next-Generation DNA Sequencing

In an alternative embodiment, diagnostic BM is used for genomic DNA extraction with a blood kit. In some cases, genomic DNA is extracted from diagnostic peripheral blood (PB). Details are found in Table S14. DNA concentration is determined with the dsDNA BR Assay Kit. Libraries are prepared following the manufacturer's protocol from 10 ng of genomic DNA using the unique molecular identifier (UMI)-based QIAseq Targeted Human Myeloid Neoplasms Panel (cat #DHS-003Z) which encompasses the exon region of 141 myeloid-related genes (Table S13).

TABLE S13

ABL1
ADA
ANKRD26
ASXL1
ASXL2
ATM
ATRX
BCL6
BCOR
BCORL1
BCR
BIRC3
BLM
BRAF
BRCA1
BRCA2
BRINP3
C17orf97
CALR
CARD11
CBL
CBLB
CBLC
CDKN2A
CEBPA
CHEK2
CREBBP
CRLF2
CSF1R
CSF3R
CTCF
CUX1
DAXX
DDX41
DNM2
DNMT1
DNMT3A
EED
EGFR
ELANE
EP300
ETNK1
ETV6
EZH2
FAM47A
FAS
FBXW7
FLRT2
FLT3
GATA1
GATA2
GJB3
GNAS
HNRNPK
HRAS
IDH1
IDH2
IKZF1
IKZF3
IL7R
JAK1
JAK2
JAK3
KAT6A
KCNA4
KCNK13
KDM6A
KDR
KIT
KLHDC8B
KLHL6
KMT2A
KMT2C
KRAS
LRRC4
LUC7L2
MAP2K1
MLH1
MPL
MSH2
MSH6
MYC
MYD88
NBN
NF1
NOTCH1
NPAT
NPM1
NRAS
NSD1
NTRK3
OR13H1
OR8B12
P2RY2
PAX5
PCDHB1
PDGFRA
PHF6
PML
PMS2
PRAMEF2
PRF1
PRPF40B
PRPF8
PTEN
PTPN11
RAD21
RB1
RELN
RUNX1
SAXO2
SETBP1
SF1
SF3A1
SF3B1
SH2B3
SH2D1A
SMARCB1
SMC1A
SMC3
SRP72
SRSF2
STAG2
STAT3
STXBP2
SUZ12
TAL1
TERC
TERT
TET2
TNFRSF13B
TP53
TPMT
TUBA3C
U2AF1
U2AF2
IS
WRN
WT1
XPO1
ZRSR2

Purified and amplified libraries are then sequenced on an Illumina NextSeq 550 system. The UMI-based variant caller smCounter2 is then used on GeneGlobe to analyse the sequencing data, which included read processing, alignment (version hg19) and calling of single nucleotide variants (SNVs)/small indels. Variant annotation is performed by ANNOVAR. Variant filtering is performed to a large extent according to the multi-step method previously described by the German AML Cooperative Group. Initially, a variant allele frequency (VAF) of 5% with a quality score of 15 is chosen as cut-off for variant filtering. Synonymous SNVs are also removed, while non-synonymous, frameshift, splicing site mutations are considered pathogenic and retained. Additionally, variants reported in OncoKB as pathogenic/likely pathogenic, oncogenic/likely oncogenic or known drivers are kept. Secondly, variants with a population frequency of ≥0.1% in the 1000 Genomes Project (Phase 3) are excluded from the analysis. Finally, variants which have a Combined Annotation Dependent Depletion (CADD) score>20 and are predicted to be functionally damaging by at least three of the following prediction tools are retained: SIFT, Polyphen_2, MutationTaster, PROVEAN. The final list of high-confidence variants is found in Table S14. In addition, Genetic Analyzer 3500 is also used to screen for NPM1, FLT3-ITD, and CEBPA mutations. For NPM1, screening involved C-terminus mutations in exon 12 and the mutation type is reported according to pre-defined criteria. All patients are screened for FLT3-ITDs using fragment analysis and Sanger Sequencing. CEBPA genotyping is performed using conventional Sanger Sequencing.

### 2.6.Cell Lines and Cell Culture

The classification involves a step of selecting GDM1, KG1a, ME1, OCI-AML3 cells and MOLM13. In a further and/or alternative embodiment, the classification involves a step of further selecting ME1 and GDM1 cells, and such two cells are maintained in RPMI-1640 medium containing 20% fetal bovine serum (FBS), while all others are maintained in RPMI-1640 medium with 10% FBS.

### 2.7.RNA Interference

In an alternative embodiment, predesigned siRNAs are used for assessing the biological significance of SPINK2 in AML by knocking down SPINK2 in KG1a cells (siRNA #1: ID_s13362, siRNA #2: ID_s224675). Negative control siRNAs are also obtained (Cat #AM4611). Approximately 5×10⁶ cells in RPMI1640 medium are transfected with 500 nM siRNAs using electroporation with 0.4 cm cuvettes and the following conditions: Voltage, 300V; capacitance, 700 μF. 48 to 72 hours after transfection, SPINK2 expression is analyzed by qPCR and Western Blot.

### 2.8.Lentiviral Transduction

GFP-labelled lentiviruses are used for assessing the biological significance of SPINK2 in AML by overexpressing SPINK2 in OCIAML3 and MOLM13 cells (pRSC—SFFV-SPINK2-E2A-Puro-E2A-GFP-Wpre) and empty vector, EV (pRSC—SFFV-Puro-E2A-GFP-wpre) are provided. Transduction is performed in approximately 2×10⁵ cells/ml at a multiplicity of infection (MOI) of 20 using Retronectin®-coated 6-well plates according to the manufacturer's instructions (Takara Bio Inc.) This is followed by puromycin selection (1 μg/ml) for at least seven days. Functional studies are then performed on cells as described and extra cells are cryopreserved.

### 2.9.Transcriptome Sequencing

Transcriptome sequencing is performed to assess the biological significance of SPINK2 in AML by comparing gene expression changes upon SPINK2 knockdown (KD) and overexpression (OE). Total RNA is extracted from two independent experiments involving KG1a cells transfected with negative control siRNA, SPINK2 siRNA #1 and SPINK2 siRNA #2 for 48 hours. Total RNA is also extracted from MOLM13 and OCIAML3 cells transduced with EV and SPINK2 lentiviruses following a 7-day puromycin selection period. All the subsequent steps involving mRNA purification from total RNA, library preparation, sequencing on the Illumina NovaSeq 6000 system, and data analysis (quality control, reference genome mapping (version hg19) and quantification of gene expression level) are performed. For quantification of gene expression levels, FPKM (Fragments Per Kilobase of transcript per Million mapped reads) of each gene is calculated based on the length of the gene and reads count mapped to this gene. Differential gene expression analysis is further performed manually by excluding non-protein coding genes and those with FPKM<1 in the control cells. Next, the FPKM of genes of the KD or OE cells is divided by the FPKM of genes of the control cells to generate the fold-change for each gene. A fold-change of 1.3 is chosen as a cut-off for both downregulation and upregulation analysis to incorporate more genes for Gene Set Enrichment Analysis (GSEA) since SPINK2 is not a transcription factor.

Quantitative RT-PCR is employed to validate selected SPINK2 target genes using the following TaqMan Gene expression assays: SLC7A11 (Hs00921938_m1), STEAP3 (Hs00217292_m1), ALCAM (Hs00977641_m1), CD86 (Hs01567026_m1), NQO1 (Hs01045993_g1), S100A9 (Hs00610058_m1), VWF (Hs01109446_m1), ITGA2B (Hs01116228_m1), IL32 (Hs00992441_m1), CCNA1 (Hs00171105_m1), HOXA6 (Hs00430615_m1), TFPI (Hs00409210_m1), CDH24 (Hs00332067_m1) and MDK (Hs00171064_m1). Each sample is measured in triplicate and gene expression is analysed by the 2-44Ct method. GAPDH is used as housekeeping gene for normalization.

### 2.10. Western Blotting

Cells are harvested, washed in Phosphate-buffered saline (PBS) and lysed using Pierce™ IP Lysis Buffer. Protein concentration is measured using Pierce™ BCA Protein Assay Kit. Approximately 30 μg of whole cell lysates are mixed with 4× Laemmli Buffer and β-mercaptoethanol and denatured for 5 minutes at 95° C. Lysates are equally loaded onto and separated using freshly prepared polyacrylamide gels. Proteins are transferred onto 0.2 μm Immun-Blot® PVDF membranes using FLASHBlot transfer buffer. The membranes are then blocked for one hour at room temperature with 5% non-fat dry milk in TBS Tween™ 20 Buffer. This is followed by incubation with primary antibodies diluted in 5% bovine serum albumin (BSA) at 4° C. overnight. Primary antibody dilutions are as follows: SPINK2 (1:1000), ALCAM (1:10000), β-ACTIN (1:10000), GAPDH (1:2500). Membranes are washed with 1× TBS Tween™ and incubated for 1 h at room temperature with species-specific horseradish peroxidase-labelled (HRP) secondary antibodies-either goat anti-rabbit IgG-HRP (Dako, #P0448) or goat anti-mouse IgG-HRP (Dako, #P0447), both at 1:2000 in 5% BSA. Chemiluminescent detection is then performed after incubation of the membranes with WesternBright ECL HRP Substrate and imaging using the ChemiDoc XRS+ System.

### 2.11. Drug Treatment and Cell Viability Assays

Cells are seeded into 96-well plates at a density of approximately 2×10⁵ cells/ml and drugs are added at indicated concentrations. Cell viability is measured at indicated time points using Cell Titer-GLO® Luminescent Cell Viability Assay. For assessment of gene expression after drug treatment, cells are seeded in 6-well plates at approximately 4×10⁵ cells/ml and drugs are added at indicated doses. RNA and/or protein is extracted 72 hours later. qPCR and Western Blot are then performed according to standard procedures to detect target gene and protein expression.

### 2.12. Statistical Analyses

In a preferred embodiment, statistical analyses are subsequently performed. GraphPad Prism could be used for such analysis. In another embodiment, various two-tailed t-tests are used for comparison of clinicopathological characteristics between patients with SPINK2^{high} and SPINK2^{low} status: Unpaired Student t-test or Mann-Whitney test or Kruskal-Wallis tests are used for continuous variables, whereas Fisher's exact test for categorical variables. For comparison of responses to standard induction among SPINK2^{high} and SPINK2^{low} groups, Fisher's exact test is used. For univariate survival analyses, Kaplan-Meier curves are generated, and the logrank P-value and logrank hazard ratio are used for comparison of groups. P-values<0.05 are considered to be statistically significant. For multivariate analysis, univariate survival analysis with Cox regression for several variables and/or combinations individually is first performed. Factors which are significantly associated with survival in the univariate analysis are then inputted into the multivariate analysis. In the multivariate analysis results, P-values<0.05 are considered statistically significant. For all other tests in the functional assays, the statistical test employed is indicated in the figure legends. The data are presented for at least two independent experiments as mean±standard deviation (SD) as indicated in figure legends.

Table S14 lists the high-confidence pathological variants identified by NGS in the adult cohort.

The present invention explained above is not limited to the aforementioned embodiment and drawings, and it will be obvious to those having an ordinary skill in the art of the prevent invention that various replacements, deformations, and changes may be made without departing from the scope of the invention.

TABLE S14
Supplementary table S14. High-confidence variant list after filtering and exclusion

| | Sample type | | | | | |
|---|---|---|---|---|---|---|
| | (BM = | | | | | |
| | bone | | | | | |
| | marrow, | | | | | |
| | PB = | | | | QUAL- | |
| Sample | peripheral | Gene | | | ITY | |
| code | blood) | name | Mutation | Impact | SCORE | VAF |
| D001 | BM | CEBPA | CEBPA:NM_001285829.1:exon1:c.568_{—} | nonframeshift | 72.88 | 0.4285714 |
| | | | 572delinsCTGCAGAA:p.E190_T191delinsLQK, CEBPA:NM_{—} | substitution | | |
| | | | 001287424.2:exon1:c.1030_1034delinsCTGCAGAA:p.E344_{—} | | | |
| | | | T345delinsLQK, CEBPA:NM_001287435.1:exon1:c.883_{—} | | | |
| | | | 887delinsCTGCAGAA:p.E295_T296delinsLQK, CEBPA:NM_{—} | | | |
| | | | 004364.5:exon1:c.925_929delinsCTGCAGAA:p.E309_{—} | | | |
| | | | T310delinsLQK | | | |
| | | CEBPA | CEBPA:NM_001287424.2:exon1:c.259_274del:p.L87Sfs*103, | frameshift | 169.16 | 0.4444444 |
| | | | CEBPA:NM_001287435.1:exon1:c.112_127del:p.L38Sfs*103, | deletion | | |
| | | | CEBPA:NM_004364.5:exon1:c.154_169del:p.L52Sfs*103 | | | |
| | | EZH2 | EZH2:NM_001203249.2:exon16:c.G1811C:p.G604A, | nonsynonymous | 26.53 | 0.1085271 |
| | | | EZH2:NM_152998.3:exon16:c.G1847C:p.G616A, | SNV | | |
| | | | EZH2:NM_001203247.2:exon17:c.G1964C:p.G655A, | | | |
| | | | EZH2:NM_001203248.2:exon17:c.G1937C:p.G646A, | | | |
| | | | EZH2:NM_004456.5:exon17:c.G1979C:p.G660A | | | |
| | | SRSF2 | SRSF2:NM_001195427.2:exon1:c.T184G:p.F62V, | nonsynonymous | 164.09 | 0.4539474 |
| | | | SRSF2:NM_003016.4:exon1:c.T184G:p.F62V | SNV | | |
| | | TET2 | TET2:NM_001127208.3:exon3:c.2840_{—} | frameshift | 24.51 | 0.1056338 |
| | | | 2841insAAAG:p.H949Kfs*24, TET2:NM_{—} | insertion | | |
| | | | 017628.4:exon3:c.2840_2841insAAAG:p.H949Kfs*24 | | | |
| D002 | BM | CEBPA | CEBPA:NM_001285829.1:exon1:c.130dupG:p.E44Gfs*7, | frameshift | 200 | 0.3125 |
| | | | CEBPA:NM_001287424.2:exon1:c.592dupG:p.E198Gfs*7, | insertion | | |
| | | | CEBPA:NM_001287435.1:exon1:c.445dupG:p.E149Gfs*7, | | | |
| | | | CEBPA:NM_004364.5:exon1:c.487dupG:p.E163Gfs*7 | | | |
| | | NPM1 | NPM1:NM_001355010.1:exon7:c.478_{—} | frameshift | 32.47 | 0.3409091 |
| | | | 479insTCTG:p.W161Cfs*12, NPM1:NM_{—} | insertion | | |
| | | | 001355007.1:exon10:c.667_668insTCTG:p.W224Cfs*12, | | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_{—} | | | |
| | | | 860insTCTG:p.W288Cfs*12 | | | |
| D003 | BM | DNMT3A | DNMT3A:NM_001320893.1:exon18:c.C2188A:p.R730S, | nonsynonymous | 200 | 0.4596003 |
| | | | DNMT3A:NM_001375819.1:exon18:c.C1975A:p.R659S, | SNV | | |
| | | | DNMT3A:NM_153759.3:exon19:c.C2077A:p.R693S, | | | |
| | | | DNMT3A:NM_022552.5:exon23:c.C2644A:p.R882S, | | | |
| | | | DNMT3A:NM_175629.2:exon23:c.C2644A:p.R882S | | | |
| | | FLT3 | FLT3:NM_004119.3:exon14:c.1793_{—} | nonframeshift | 47.71 | 0.079646 |
| | | | 1794insCTACGTTGATTTCAGAGAATATGA:p.Y597_{—} | insertion | | |
| | | | E598insDYVDFREY | | | |
| | | NPM1 | NPM1:NM_001355010.1:exon7:c.479_{—} | frameshift | 67.01 | 0.3855422 |
| | | | 480insCTGC:p.W161Cfs*12, | insertion | | |
| | | | NPM1:NM_001355007.1:exon10:c.668_{—} | | | |
| | | | 669insCTGC:p.W224Cfs*12, | | | |
| | | | NPM1:NM_199185.3:exon10:c.773_774insCTGC:p.W259Cfs*12, | | | |
| | | | NPM1:NM_002520.7:exon11:c.860_861insCTGC:p.W288Cfs*12, | | | |
| | | | NPM1:NM_001355006.1:exon12:c.860_{—} | | | |
| | | | 861insCTGC:p.W288Cfs*12 | | | |
| | | RELN | RELN:NM_005045.4:exon40:c.G6032A:p.R2011H, RELN:NM_{—} | nonsynonymous | 200 | 0.4923858 |
| | | | 173054.2:exon40:c.G6032A:p.R2011H | SNV | | |
| D004 | BM | CEBPA | CEBPA:NM_001285829.1:exon1:c.C127T:p.Q43X, CEBPA:NM_{—} | stopgain | 127.53 | 0.4078947 |
| | | | 001287424.2:exon1:c.C589T:p.Q197X, CEBPA:NM_{—} | | | |
| | | | 001287435.1:exon1:c.C442T:p.Q148X, | | | |
| | | | CEBPA:NM_004364.5:exon1:c.C484T:p.Q162X | | | |
| | | FLT3 | FLT3:NM_004119.3:exon14:c.1795_1796ins | nonframeshift | 112.53 | 0.3056995 |
| | | | TTGATTTCAGAGAATATGAAT:p.E598_Y599insFDFREYE | insertion | | |
| | | WT1 | WT1:NM_001367854.1:exon5:c.T184C:p.C62R, | nonsynonymous | 18.92 | 0.0702703 |
| | | | WT1:NM_000378.6:exon8:c.T1321C:p.C441R, | SNV | | |
| | | | WT1:NM_001198552.2:exon8:c.T670C:p.C224R, | | | |
| | | | WT1:NM_001198551.1:exon9:c.T721C:p.C241R, | | | |
| | | | WT1:NM_024424.5:exon9:c.T1372C:p.C458R, | | | |
| | | | WT1:NM_024426.6:exon9:c.T1372C:p.C458R | | | |
| D006 | BM | BRCA1 | BRCA1:NM_007297.4:exon15:c.A4927C:p.K1643Q, | nonsynonymous | 78.36 | 0.4507042 |
| | | | BRCA1:NM_007298.3:exon15:c.A1756C:p.K586Q, | SNV | | |
| | | | BRCA1:NM_007294.4:exon16:c.A5068C:p.K1690Q, | | | |
| | | | BRCA1:NM_007299.4:exon16:c.A1756C:p.K586Q, | | | |
| | | | BRCA1:NM_007300.4:exon17:c.A5131C:p.K1711Q | | | |
| | | CEBPA | CEBPA:NM_001287424.2:exon1:c.169_179del:p.P57Afs*82, | frameshift | 28.22 | 0.1807229 |
| | | | CEBPA:NM_001287435.1:exon1:c.22_32del:p.P8Afs*82, | deletion | | |
| | | | CEBPA:NM_004364.5:exon1:c.64_74del:p.P22Afs*82 | | | |
| D007 | BM | NRAS | NRAS:NM_002524.5:exon2:c.G38A:p.G13D | nonsynonymous | 200 | 0.3225191 |
| | | | | SNV | | |
| D008 | BM | CEBPA | CEBPA:NM_001285829.1:exon1:c.670delC:p.R224Afs*79, | frameshift | 200 | 0.4151625 |
| | | | CEBPA:NM_001287424.2:exon1:c.1132delC:p.R378Afs*79, | deletion | | |
| | | | CEBPA:NM_001287435.1:exon1:c.985delC:p.R329Afs*79, | | | |
| | | | CEBPA:NM_004364.5:exon1:c.1027delC:p.R343Afs*79 | | | |
| | | NPM1 | NPM1:NM_001355010.1:exon7:c.478_479insTCTG:p.W161Cfs*12, | frameshift | 84.77 | 0.3559322 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | TET2 | TET2:NM_001127208.3:exon3:c.1308_1311del:p.Y437Pfs*9, | frameshift | 182.81 | 0.3755102 |
| | | | TET2:NM_017628.4:exon3:c.1308_1311del:p.Y437Pfs*9 | deletion | | |
| | | TET2 | TET2:NM_001127208.3:exon3:c.1968_1969del:p.S657Tfs*23, | frameshift | 200 | 0.507772 |
| | | | TET2:NM_017628.4:exon3:c.1968_1969del:p.S657Tfs*23 | deletion | | |
| D009 | BM | DNMT3A | DNMT3A:NM_001320893.1:exon18:c.G2189A:p.R730H, | nonsynonymous | 139.5 | 0.4342105 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | EP300 | EP300:NM_001362843.2:exon30:c.7160_7164del:p.H2388Efs*32, | frameshift | 102.84 | 0.4363636 |
| | | | EP300:NM_001429.4:exon31:c.7238_7242del:p.H2414Efs*32 | deletion | | |
| | | NPM1 | NPM1:NM_001355010.1:exon7:c.478_479insTCTG:p.W161Cfs*12, | frameshift | 30.66 | 0.4333333 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | PTPN11 | PTPN11:NM_001330437.2:exon13:c.G1542T:p.Q514H, | nonsynonymous | 15.08 | 0.0761905 |
| | | | PTPN11:NM_001374625.1:exon13:c.G1527T:p.Q509H, | SNV | | |
| | | | PTPN11:NM_002834.5:exon13:c.G1530T:p.Q510H | | | |
| | | TET2 | TET2:NM_001127208.3:exon3:c.C2872T:p.Q958X, | stopgain | 62.88 | 0.4666667 |
| | | | TET2:NM_017628.4:exon3:c.C2872T:p.Q958X | | | |
| D010 | BM | BCOR | BCOR:NM_001123384.2:exon11:c.C4483T:p.R1495X, | stopgain | 200 | 0.8448276 |
| | | | BCOR:NM_001123383.1:exon12:c.C4537T:p.R1513X, | | | |
| | | | BCOR:NM_001123385.2:exon12:c.C4639T:p.R1547X, | | | |
| | | | BCOR:NM_017745.6:exon12:c.C4537T:p.R1513X | | | |
| | | BCORL1 | BCORL1:NM_021946.5:exon13:c.5036dupC:p.G1682Rfs*4, | frameshift | 200 | 0.8723404 |
| | | | BCORL1:NM_001379450.1:exon14:c.5258dupC:p.G1756Rfs*4, | insertion | | |
| | | | BCORL1:NM_001379451.1:exon14:c.5258dupC:p.G1756Rfs*4, | | | |
| | | | BCORL1:NM_001184772.3:exon15:c.5258dupC:p.G1756Rfs*4 | | | |
| | | IDH1 | IDH1:NM_001282386.1:exon4:c.C394T:p.R132C, | nonsynonymous | 179.29 | 0.624 |
| | | | IDH1:NM_001282387.1:exon4:c.C394T:p.R132C, | SNV | | |
| | | | IDH1:NM_005896.4:exon4:c.C394T:p.R132C | | | |
| | | NRAS | NRAS:NM_002524.5:exon2:c.G34A:p.G12S | nonsynonymous | 200 | 0.4642857 |
| | | | | SNV | | |
| D011 | BM | CEBPA | CEBPA:NM_001285829.1:exon1:c.559_560insAGC:p.Q186_{—} | nonframeshift | 95.1 | 0.4444444 |
| | | | R187insQ, CEBPA:NM_001287424.2:exon1:c.1021_{—} | insertion | | |
| | | | 1022insAGC:p.Q340_R341insQ, CEBPA:NM_{—} | | | |
| | | | 001287435.1:exon1:c.874_875insAGC:p.Q291_R292insQ, | | | |
| | | | CEBPA:NM_004364.5:exon1:c.916_917insAGC:p.Q305_R306insQ | | | |
| | | CEBPA | CEBPA:NM_001287424.2:exon1:c.249_250delinsT:p.P84Rfs*111, | frameshift | 172.57 | 0.4576271 |
| | | | CEBPA:NM_001287435.1:exon1:c.102_103delinsT:p.P35Rfs*111, | substitution | | |
| | | | CEBPA:NM_004364.5:exon1:c.144_145delinsT:p.P49Rfs*111 | | | |
| | | GATA2 | GATA2:NM_001145662.1:exon4:c.C953T:p.A318V, | nonsynonymous | 98.27 | 0.3164557 |
| | | | GATA2:NM_032638.5:exon4:c.C953T:p.A318V, | SNV | | |
| | | | GATA2:NM_001145661.2:exon5:c.C953T:p.A318V | | | |
| D012 | BM | DNMT3A | DNMT3A:NM_001320893.1:exon18:c.C2188T:p.R730C, | nonsynonymous | 200 | 0.4918699 |
| | | | DNMT3A:NM_001375819.1:exon18:c.C1975T:p.R659C, | SNV | | |
| | | | DNMT3A:NM_153759.3:exon19:c.C2077T:p.R693C, | | | |
| | | | DNMT3A:NM_022552.5:exon23:c.C2644T:p.R882C, | | | |
| | | | DNMT3A:NM_175629.2:exon23:c.C2644T:p.R882C | | | |
| | | FLT3 | FLT3:NM_004119.3:exon20:c.G2503C:p.D835H | nonsynonymous | 19.12 | 0.0934579 |
| | | | | SNV | | |
| | | KCNA4 | KCNA4:NM_002233.4:exon2:c.G1427A:p.R476Q | nonsynonymous | 148.74 | 0.55 |
| | | | | SNV | | |
| | | KMT2A | KMT2A:NM_001197104.2:exon27:c.A8482C:p.N2828H, | nonsynonymous | 170.17 | 0.5267176 |
| | | | KMT2A:NM_005933.4:exon27:c.A8473C:p.N2825H | SNV | | |
| | | NPM1 | NPM1:NM_001355010.1:exon7:c.478_479insTCTG:p.W161Cfs*12, | frameshift | 24.57 | 0.3142857 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | NRAS | NRAS:NM_002524.5:exon2:c.G34A:p.G12S | nonsynonymous | 49.86 | 0.1891892 |
| | | | | SNV | | |
| | | PCDHB1 | PCDHB1:NM_013340.4:exon1:c.G1901C:p.R634T | nonsynonymous | 172.53 | 0.5263158 |
| | | | | SNV | | |
| | | SMC1A | SMC1A:NM_006306.4:exon11:c.G1757A:p.R586Q, | nonsynonymous | 66.29 | 0.5714286 |
| | | | SMC1A:NM_001281463.1:exon12:c.G1691A:p.R564Q | SNV | | |
| D013 | BM | RUNX1 | RUNX1:NM_001001890.3:exon2:c.A301C:p.T101P, | nonsynonymous | 191.77 | 0.9558824 |
| | | | RUNX1:NM_001122607.2:exon2:c.A301C:p.T101P, | SNV | | |
| | | | RUNX1:NM_001754.5:exon5:c.A382C:p.T128P | | | |
| D014 | BM | ASXL1 | ASXL1:NM_001363734.1:exon11:c.C2014T:p.Q672X, | stopgain | 142.34 | 0.4680851 |
| | | | ASXL1:NM_015338.6:exon12:c.C2197T:p.Q733X | | | |
| | | DNMT3A | DNMT3A:NM_001320893.1:exon18:c.G2189A:p.R730H, | nonsynonymous | 200 | 0.4854369 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | IDH2 | IDH2:NM_001290114.2:exon2:c.G29A:p.R10Q, | nonsynonymous | 110.63 | 0.5326087 |
| | | | IDH2:NM_001289910.1:exon4:c.G263A:p.R88Q, | SNV | | |
| | | | IDH2:NM_002168.4:exon4:c.G419A:p.R140Q | | | |
| | | RUNX1 | RUNX1:NM_001001890.3:exon1:c.228_229insCT:p.T77Lfs*19, | frameshift | 200 | 0.9677419 |
| | | | RUNX1:NM_001122607.2:exon1:c.228_229insCT:p.T77Lfs*19, | insertion | | |
| | | | RUNX1:NM_001754.5:exon4:c.309_310insCT:p.T104Lfs*19 | | | |
| D015 | BM | CEBPA | CEBPA:NM_001285829.1:exon1:c.560_577del:p.R187_Q192del, | nonframeshift | 91.56 | 0.3543307 |
| | | | CEBPA:NM_001287424.2:exon1:c.1022_1039del:p.R341_Q346del, | deletion | | |
| | | | CEBPA:NM_001287435.1:exon1:c.875_892del:p.R292_Q297del, | | | |
| | | | CEBPA:NM_004364.5:exon1:c.917_934del:p.R306_Q311del | | | |
| | | CEBPA | CEBPA:NM_001287424.2:exon1:c.400_410del:p.G134Rfs*5, | frameshift | 179.3 | 0.6 |
| | | | CEBPA:NM_001287435.1:exon1:c.253_263del:p.G85Rfs*5, | deletion | | |
| | | | CEBPA:NM_004364.5:exon1:c.295_305del:p.G99Rfs*5 | | | |
| | | CSF3R | CSF3R:NM_000760.4:exon14:c.C1853T:p.T618I, | nonsynonymous | 26.69 | 0.0977011 |
| | | | CSF3R:NM_156039.3:exon14:c.C1853T:p.T618I, | SNV | | |
| | | | CSF3R:NM_172313.3:exon14:c.C1853T:p.T618I | | | |
| | | EP300 | EP300:NM_001362843.2:exon10:c.A1916G:p.K639R, | nonsynonymous | 36.68 | 0.4210526 |
| | | | EP300:NM_001429.4:exon10:c.A1916G:p.K639R | SNV | | |
| | | GATA2 | GATA2:NM_001145662.1:exon4:c.A970G:p.K324E, | nonsynonymous | 200 | 0.5219298 |
| | | | GATA2:NM_032638.5:exon4:c.A970G:p.K324E, | SNV | | |
| | | | GATA2:NM_001145661.2:exon5:c.A970G:p.K324E | | | |
| D016 | BM | KRAS | KRAS:NM_001369786.1:exon2:c.G38A:p.G13D, | nonsynonymous | 111.18 | 0.1981982 |
| | | | KRAS:NM_001369787.1:exon2:c.G38A:p.G13D, | SNV | | |
| | | | KRAS:NM_004985.5:exon2:c.G38A:p.G13D, | | | |
| | | | KRAS:NM_033360.4:exon2:c.G38A:p.G13D | | | |
| | | NRAS | NRAS:NM_002524.5:exon2:c.G35A:p.G12D | nonsynonymous | 26.46 | 0.0566038 |
| | | | | SNV | | |
| D017 | BM | DNMT3A | DNMT3A:NM_001320893.1:exon15:c.1910_{—} | nonframeshift | 81.97 | 0.2978723 |
| | | | 1911insTGCACA:p.H637_R638insAH, DNMT3A:NM_{—} | insertion | | |
| | | | 001375819.1:exon15:c.1697_1698insTGCACA:p.H566_{—} | | | |
| | | | R567insAH, DNMT3A:NM_153759.3:exon16:c.1799_{—} | | | |
| | | | 1800insTGCACA:p.H600_R601insAH, | | | |
| | | | DNMT3A:NM_022552.5:exon20:c.2366_{—} | | | |
| | | | 2367insTGCACA:p.H789_R790insAH, DNMT3A:NM_{—} | | | |
| | | | 175629.2:exon20:c.2366_2367insTGCACA:p.H789_{—} | | | |
| | | | R790inSAH | | | |
| | | IDH2 | IDH2:NM_001290114.2:exon2:c.G29A:p.R10Q, | nonsynonymous | 128.86 | 0.4836066 |
| | | | IDH2:NM_001289910.1:exon4:c.G263A:p.R88Q, | SNV | | |
| | | | IDH2:NM_002168.4:exon4:c.G419A:p.R140Q | | | |
| | | RUNX1 | RUNX1:NM_001001890.3:exon6:c.964dupT:p.Y322Lfs*251, | frameshift | 113.63 | 0.4153846 |
| | | | RUNX1:NM_001754.5:exon9:c.1045dupT:p.Y349Lfs*251 | insertion | | |
| D018 | BM | EZH2 | EZH2:NM_152998.3:exon9:c.1000_1001insCGTGC:p.L334Pfs*48, | frameshift | 56.12 | 0.2125 |
| | | | EZH2:NM_001203247.2:exon10:c.1117_{—} | insertion | | |
| | | | 1118insCGTGC:p.L373Pfs*48, EZH2:NM_{—} | | | |
| | | | 001203248.2:exon10:c.1090_1091insCGTGC:p.L364Pfs*48, | | | |
| | | | EZH2:NM_001203249.2:exon10:c.1090_{—} | | | |
| | | | 1091insCGTGC:p.L364Pfs*48, EZH2:NM_{—} | | | |
| | | | 004456.5:exon10:c.1132_1133insCGTGC:p.L378Pfs*48 | | | |
| | | EZH2 | EZH2:NM_001203249.2:exon19:c.2029_{—} | frameshift | 200 | 0.2481618 |
| | | | 2030insGGGAT:p.Y677Wfs*9, EZH2:NM_152998.3:exon19:c.2065_{—} | insertion | | |
| | | | 2066insGGGAT:p.Y689Wfs*9, EZH2:NM_{—} | | | |
| | | | 001203247.2:exon20:c.2182_2183insGGGAT:p.Y728Wfs*9, | | | |
| | | | EZH2:NM_001203248.2:exon20:c.2155_{—} | | | |
| | | | 2156insGGGAT:p.Y719Wfs*9, EZH2:NM_{—} | | | |
| | | | 004456.5:exon20:c.2197_2198insGGGAT:p.Y733Wfs*9 | | | |
| | | SETBP1 | SETBP1:NM_001379141.1:exon4:c.A2077G: | nonsynonymous | 95.64 | 0.4747475 |
| | | | p.K693E, SETBP1:NM_001379142.1:exon4:c. | SNV | | |
| | | | A2077G:p.K693E, SETBP1:NM_015559.3:ex | | | |
| | | | on4:c.A2077G:p.K693E | | | |
| D019 | BM | DNMT3A | DNMT3A:NM_001320893.1:exon4:c.T560G:p.V187G, | nonsynonymous | 131.24 | 0.4413793 |
| | | | DNMT3A:NM_001375819.1:exon4:c.T347G:p.V116G, | SNV | | |
| | | | DNMT3A:NM_153759.3:exon5:c.T449G:p.V150G, | | | |
| | | | DNMT3A:NM_022552.5:exon9:c.T1016G:p.V339G, | | | |
| | | | DNMT3A:NM_175629.2:exon9:c.T1016G:p.V339G | | | |
| | | DNMT3A | DNMT3A:NM_001320893.1:exon18:c.G2227A:p.V743M, | nonsynonymous | 200 | 0.4409938 |
| | | | DNMT3A:NM_001375819.1:exon18:c.G2014A:p.V672M, | SNV | | |
| | | | DNMT3A:NM_153759.3:exon19:c.G2116A:p.V706M, | | | |
| | | | DNMT3A:NM_022552.5:exon23:c.G2683A:p.V895M, | | | |
| | | | DNMT3A:NM_175629.2:exon23:c.G2683A:p.V895M | | | |
| | | FBXW7 | FBXW7:NM_033632.3:exon2:c.G22C:p.V8L, | nonsynonymous | 48.9 | 0.4035088 |
| | | | FBXW7:NM_001257069.1:exon4:c.G22C:p.V8L, | SNV | | |
| | | | FBXW7:NM_001349798.2:exon4:c.G22C:p.V8L | | | |
| | | NPM1 | NPM1:NM_001355010.1:exon7:c.478_479insTCTG:p.W161Cfs*12, | frameshift | 38.6 | 0.3529412 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D020 | BM | CEBPA | CEBPA:NM_001287424.2:exon1:c.424_427del:p.D142Tfs*52, | frameshift | 128.93 | 0.4246575 |
| | | | CEBPA:NM_001287435.1:exon1:c.277_280del:p.D93Tfs*52, | deletion | | |
| | | | CEBPA:NM_004364.5:exon1:c.319_322del:p.D107Tfs*52 | | | |
| | | CEBPA | CEBPA:NM_001285829.1:exon1:c.550_558del:p.A184_Q186del, | nonframeshift | 200 | 0.4207493 |
| | | | CEBPA:NM_001287424.2:exon1:c.1012_1020del:p.A338_Q340del, | deletion | | |
| | | | CEBPA:NM_001287435.1:exon1:c.865_873del:p.A289_Q291del, | | | |
| | | | CEBPA:NM_004364.5:exon1:c.907_915del:p.A303_Q305del | | | |
| | | CSF3R | CSF3R:NM_000760.4:exon14:c.C1853T:p.T618I, | nonsynonymous | 109.94 | 0.1501057 |
| | | | CSF3R:NM_156039.3:exon14:c.C1853T:p.T618I, | SNV | | |
| | | | CSF3R:NM_172313.3:exon14:c.C1853T:p.T618I | | | |
| | | DNM2 | DNM2:NM_001005362.2:exon15:c.1667_1669del:p.K558del, | nonframeshift | 91.1 | 0.1237525 |
| | | | DNM2:NM_004945.3:exon15:c.1667_1669del:p.K558del, | deletion | | |
| | | | DNM2:NM_001005360.3:exon16:c.1679_1681del:p.K562del, | | | |
| | | | DNM2:NM_001005361.3:exon16:c.1679_1681del:p.K562del, | | | |
| | | | DNM2:NM_001190716.2:exon16:c.1679_1681del:p.K562del | | | |
| | | GATA2 | GATA2:NM_001145662.1:exon4:c.C953T:p.A318V, | nonsynonymous | 54.71 | 0.0696203 |
| | | | GATA2:NM_032638.5:exon4:c.C953T:p.A318V, | SNV | | |
| | | | GATA2:NM_001145661.2:exon5:c.C953T:p.A318V | | | |
| | | KRAS | KRAS:NM_001369786.1:exon2:c.G38A:p.G13D, | nonsynonymous | 34.74 | 0.0734463 |
| | | | KRAS:NM_001369787.1:exon2:c.G38A:p.G13D, | SNV | | |
| | | | KRAS:NM_004985.5:exon2:c.G38A:p.G13D, | | | |
| | | | KRAS:NM_033360.4:exon2:c.G38A:p.G13D | | | |
| D021 | BM | DNMT1 | DNMT1:NM_001130823.3:exon3:c.G151A:p.E51K, | nonsynonymous | 200 | 0.4966102 |
| | | | DNMT1:NM_001318730.2:exon3:c.G151A:p.E51K, | SNV | | |
| | | | DNMT1:NM_001379.4:exon3:c.G151A:p.E51K | | | |
| | | KIT | KIT:NM_000222.3:exon17:c.A2447T:p.D816V, | nonsynonymous | 47.42 | 0.2061069 |
| | | | KIT:NM_001093772.2:exon17:c.A2435T:p.D812V, | SNV | | |
| | | | KIT:NM_001385284.1:exon17:c.A2450T:p.D817V, | | | |
| | | | KIT:NM_001385285.1:exon17:c.A2444T:p.D815V, | | | |
| | | | KIT:NM_001385286.1:exon17:c.A2432T:p.D811V, | | | |
| | | | KIT:NM_001385288.1:exon17:c.A2438T:p.D813V, | | | |
| | | | KIT:NM_001385290.1:exon17:c.A2447T:p.D816V, | | | |
| | | | KIT:NM_001385292.1:exon17:c.A2435T:p.D812V | | | |
| | | TET2 | TET2:NM_001127208.3:exon3:c.1165_1166insT:p.K389Ifs*54, | frameshift | 200 | 0.9219858 |
| | | | TET2:NM_017628.4:exon3:c.1165_1166insT:p.K389Ifs*54 | insertion | | |
| D022 | BM | MLH1 | MLH1:NM_001167619.2:exon9:c.G71A:p.R24H, | nonsynonymous | 153.69 | 0.4534161 |
| | | | MLH1:NM_001258273.1:exon9:c.G71A:p.R24H, | SNV | | |
| | | | MLH1:NM_001354615.1:exon9:c.G71A:p.R24H, | | | |
| | | | MLH1:NM_001354616.1:exon9:c.G71A:p.R24H, | | | |
| | | | MLH1:NM_001354629.1:exon9:c.G695A:p.R232H, | | | |
| | | | MLH1:NM_000249.4:exon10:c.G794A:p.R265H, | | | |
| | | | MLH1:NM_001167617.2:exon10:c.G500A:p.R167H, | | | |
| | | | MLH1:NM_001167618.2:exon10:c.G71A:p.R24H, | | | |
| | | | MLH1:NM_001258271.1:exon10:c.G794A:p.R265H, | | | |
| | | | MLH1:NM_001354617.1:exon10:c.G71A:p.R24H, | | | |
| | | | MLH1:NM_001354618.1:exon10:c.G71A:p.R24H, | | | |
| | | | MLH1:NM_001354620.1:exon10:c.G500A:p.R167H, | | | |
| | | | MLH1:NM_001354628.1:exon10:c.G794A:p.R265H, | | | |
| | | | MLH1:NM_001354630.1:exon10:c.G794A:p.R265H, | | | |
| | | | MLH1:NM_001258274.2:exon11:c.G71A:p.R24H, | | | |
| | | | MLH1:NM_001354619.1:exon11:c.G71A:p.R24H | | | |
| | | NRAS | NRAS:NM_002524.5:exon2:c.G34A:p.G12S | nonsynonymous | 194.01 | 0.3726236 |
| | | | | SNV | | |
| D023 | BM | NRAS | NRAS:NM_002524.5:exon2:c.G34A:p.G12S | nonsynonymous | 33.51 | 0.0503472 |
| | | | | SNV | | |
| | | NRAS | NRAS:NM_002524.5:exon3:c.A182G:p.Q61R | nonsynonymous | 170.1 | 0.2884013 |
| | | | | SNV | | |
| D024 | BM | CREBBP | CREBBP:NM_001079846.1:exon20:c.3717delC:p.E1240Nfs*35, | frameshift | 121.28 | 0.4296296 |
| | | | CREBBP:NM_004380.3:exon21:c.3831delC:p.E1278Nfs*35 | deletion | | |
| | | IKZF1 | IKZF1:NM_001220767.2:exon3:c.A278C:p.Y93S, | nonsynonymous | 200 | 0.453125 |
| | | | IKZF1:NM_001220770.2:exon3:c.A278C:p.Y93S, | SNV | | |
| | | | IKZF1:NM_001220768.2:exon4:c.A539C:p.Y180S, | | | |
| | | | IKZF1:NM_001291838.2:exon4:c.A278C:p.Y93S, | | | |
| | | | IKZF1:NM_001291839.2:exon4:c.A278C:p.Y93S, | | | |
| | | | IKZF1:NM_001220765.3:exon5:c.A539C:p.Y180S, | | | |
| | | | IKZF1:NM_001291837.2:exon5:c.A539C:p.Y180S, | | | |
| | | | IKZF1:NM_006060.6:exon5:c.A539C:p.Y180S | | | |
| | | NRAS | NRAS:NM_002524.5:exon3:c.C181A:p.Q61K | nonsynonymous | 154.4 | 0.5503876 |
| | | | | SNV | | |
| D025 | BM | KRAS | KRAS:NM_001369786.1:exon2:c.G35A:p.G12D, | nonsynonymous | 72.39 | 0.2657343 |
| | | | KRAS:NM_001369787.1:exon2:c.G35A:p.G12D, | SNV | | |
| | | | KRAS:NM_004985.5:exon2:c.G35A:p.G12D, | | | |
| | | | KRAS:NM_033360.4:exon2:c.G35A:p.G12D | | | |
| D026 | BM | CEBPA | CEBPA:NM_001287424.2:exon1:c.184dupA:p.S62Kfs*81, | frameshift | 126.76 | 0.3188406 |
| | | | CEBPA:NM_001287435.1:exon1:c.37dupA:p.S13Kfs*81, | insertion | | |
| | | | CEBPA:NM_004364.5:exon1:c.79dupA:p.S27Kfs*81 | | | |
| | | CEBPA | CEBPA:NM_001285829.1:exon1:c.571_576del:p.T191_Q192del, | nonframeshift | 200 | 0.5644172 |
| | | | CEBPA:NM_001287424.2:exon1:c.1033_1038del:p.T345_Q346del, | deletion | | |
| | | | CEBPA:NM_001287435.1:exon1:c.886_891del:p.T296_Q297del, | | | |
| | | | CEBPA:NM_004364.5:exon1:c.928_933del:p.T310_Q311del | | | |
| | | NSD1 | NSD1:NM_022455.5:exon18:c.C5854T:p.R1952W, | nonsynonymous | 23.11 | 0.0761905 |
| | | | NSD1:NM_001365684.1:exon19:c.C5047T:p.R1683W, | SNV | | |
| | | | NSD1:NM_172349.2:exon19:c.C5047T:p.R1683W | | | |
| | | SF1 | SF1:NM_001346409.2:exon9:c.C848T:p.P283L, | nonsynonymous | 200 | 0.4779116 |
| | | | SF1:NM_001346410.2:exon9:c.C848T:p.P283L, | SNV | | |
| | | | SF1:NM_001178030.2:exon10:c.C1568T:p.P523L, | | | |
| | | | SF1:NM_001178031.3:exon10:c.C1115T:p.P372L, | | | |
| | | | SF1:NM_001346363.2:exon10:c.C1193T:p.P398L, | | | |
| | | | SF1:NM_001346364.2:exon10:c.C1193T:p.P398L, | | | |
| | | | SF1:NM_001378956.1:exon10:c.C1568T:p.P523L, | | | |
| | | | SF1:NM_001378957.1:exon10:c.C1568T:p.P523L, | | | |
| | | | SF1:NM_004630.4:exon10:c.C1193T:p.P398L, | | | |
| | | | SF1:NM_201995.3:exon10:c.C1193T:p.P398L, | | | |
| | | | SF1:NM_201997.3:exon10:c.C1193T:p.P398L, | | | |
| | | | SF1:NM_201998.3:exon10:c.C1193T:p.P398L | | | |
| D027 | BM | NPM1 | NPM1:NM_001355010.1:exon7:c.480_481insTGCA:p.W161Cfs*12, | frameshift | 90.4 | 0.4174757 |
| | | | NPM1:NM_001355007.1:exon10:c.669_670insTGCA:p.W224Cfs*12, | insertion | | |
| | | | NPM1:NM_199185.3:exon10:c.774_775insTGCA:p.W259Cfs*12, | | | |
| | | | NPM1:NM_002520.7:exon11:c.861_862insTGCA:p.W288Cfs*12, | | | |
| | | | NPM1:NM_001355006.1:exon12:c.861_862insTGCA:p.W288Cfs*12 | | | |
| | | RUNX1 | RUNX1:NM_001001890.3:exon2:c.286dupG:p.D96Gfs*15, | frameshift | 160.24 | 0.4578313 |
| | | | RUNX1:NM_001122607.2:exon2:c.286dupG:p.D96Gfs*15, | insertion | | |
| | | | RUNX1:NM_001754.5:exon5:c.367dupG:p.D123Gfs*15 | | | |
| D028 | BM | IDH2 | IDH2:NM_001290114.2:exon2:c.G125A:p.R42K, | nonsynonymous | 200 | 0.4401114 |
| | | | IDH2:NM_001289910.1:exon4:c.G359A:p.R120K, | SNV | | |
| | | | IDH2:NM_002168.4:exon4:c.G515A:p.R172K | | | |
| | | NRAS | NRAS:NM_002524.5:exon2:c.G34A:p.G12S | nonsynonymous | 20.57 | 0.1670702 |
| | | | | SNV | | |
| | | STAG2 | STAG2:NM_001375375.1:exon17:c.1702delG:p.A568Pfs*8, | frameshift | 200 | 0.4456522 |
| | | | STAG2:NM_006603.5:exon17:c.1702delG:p.A568Pfs*8, | deletion | | |
| | | | STAG2:NM_001042749.2:exon18:c.1702delG:p.A568Pfs*8, | | | |
| | | | STAG2:NM_001042750.2:exon18:c.1702delG:p.A568Pfs*8, | | | |
| | | | STAG2:NM_001042751.2:exon18:c.1702delG:p.A568Pfs*8, | | | |
| | | | STAG2:NM_001282418.2:exon18:c.1702delG:p.A568Pfs*8 | | | |
| D029 | BM | ETV6 | ETV6:NM_001987.5:exon6:c.T1073G:p.I358S | nonsynonymous | 162.12 | 0.2235023 |
| | | | | SNV | | |
| | | IKZF1 | IKZF1:NM_001220767.2:exon3:c.A215G:p.N72S, | nonsynonymous | 200 | 0.5241636 |
| | | | IKZF1:NM_001220770.2:exon3:c.A215G:p.N72S, | SNV | | |
| | | | IKZF1:NM_001220768.2:exon4:c.A476G:p.N159S, | | | |
| | | | IKZF1:NM_001291838.2:exon4:c.A215G:p.N72S, | | | |
| | | | IKZF1:NM_001291839.2:exon4:c.A215G:p.N72S, | | | |
| | | | IKZF1:NM_001220765.3:exon5:c.A476G:p.N159S, | | | |
| | | | IKZF1:NM_001291837.2:exon5:c.A476G:p.N159S, | | | |
| | | | IKZF1:NM_006060.6:exon5:c.A476G:p.N159S | | | |
| | | KRAS | KRAS:NM_001369786.1:exon4:c.A351C:p.K117N, | nonsynonymous | 43.8 | 0.1386139 |
| | | | KRAS:NM_001369787.1:exon4:c.A351C:p.K117N, | SNV | | |
| | | | KRAS:NM_004985.5:exon4:c.A351C:p.K117N, | | | |
| | | | KRAS:NM_033360.4:exon4:c.A351C:p.K117N | | | |
| | | TAL1 | TAL1:NM_001290406.2:exon3:c.G109A:p.E37K, | nonsynonymous | 104.8 | 0.1987179 |
| | | | TAL1:NM_001287347.2:exon5:c.G586A:p.E196K, | SNV | | |
| | | | TAL1:NM_001290403.1:exon5:c.G586A:p.E196K, | | | |
| | | | TAL1:NM_001290405.1:exon5:c.G586A:p.E196K, | | | |
| | | | TAL1:NM_001290404.1:exon6:c.G586A:p.E196K, | | | |
| | | | TAL1:NM_003189.5:exon6:c.G586A:p.E196K | | | |
| D030 | BM | NPM1 | NPM1:NM_001355010.1:exon7:c.478_479insTCTG:p.W161Cfs*12, | frameshift | 36.45 | 0.4102564 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | TET2 | TET2:NM_001127208.3:exon11:c.G5738A:p.G1913D | nonsynonymous | 122.83 | 0.4041096 |
| | | | | SNV | | |
| | | TET2 | TET2:NM_001127208.3:exon6:c.C3781T:p.R1261C | nonsynonymous | 184.77 | 0.4554974 |
| | | | | SNV | | |
| D031 | BM | KRAS | KRAS:NM_001369786.1:exon2:c.G38A:p.G13D, | nonsynonymous | 149.47 | 0.3085938 |
| | | | KRAS:NM_001369787.1:exon2:c.G38A:p.G13D, | SNV | | |
| | | | KRAS:NM_004985.5:exon2:c.G38A:p.G13D, | | | |
| | | | KRAS:NM_033360.4:exon2:c.G38A:p.G13D | | | |
| D032 | BM | ATM | ATM:NM_000051.4:exon54:c.T7934G:p.I2645R, | nonsynonymous | 185.1 | 0.5408805 |
| | | | ATM:NM_001351834.2:exon55:c.T7934G:p.I2645R | SNV | | |
| | | KIT | KIT:NM_000222.3:exon8:c.1248delG:p.T417Lfs*6, | frameshift | 97.92 | 0.25 |
| | | | KIT:NM_001093772.2:exon8:c.1248de1G:p.T417Lfs*6, | deletion | | |
| | | | KIT:NM_001385284.1:exon8:c.1251delG:p.T418Lfs*6, | | | |
| | | | KIT:NM_001385285.1:exon8:c.1248delG:p.T417Lfs*6, | | | |
| | | | KIT:NM_001385286.1:exon8:c.1248delG:p.T417Lfs*6, | | | |
| | | | KIT:NM_001385288.1:exon8:c.1251delG:p.T418Lfs*6, | | | |
| | | | KIT:NM_001385290.1:exon8:c.1251delG:p.T418Lfs*6, | | | |
| | | | KIT:NM_001385292.1:exon8:c.1251delG:p.T418Lfs*6 | | | |
| D033 | BM | DNMT3A | DNMT3A:NM_001320893.1:exon18:c.G2189A:p.R730H, | nonsynonymous | 200 | 0.4381847 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | EP300 | EP300:NM_001362843.2:exon13:c.C2461T:p.P821S, | nonsynonymous | 200 | 0.4705882 |
| | | | EP300:NM_001429.4:exon14:c.C2539T:p.P847S | SNV | | |
| | | PDGFRA | PDGFRA:NM_001347827.2:exon4:c.C370A:p.P124T, | nonsynonymous | 200 | 0.4761905 |
| | | | PDGFRA:NM_001347829.2:exon4:c.C370A:p.P124T, | SNV | | |
| | | | PDGFRA:NM_001347830.1:exon4:c.C409A:p.P137T, | | | |
| | | | PDGFRA:NM_006206.6:exon4:c.C370A:p.P124T, | | | |
| | | | PDGFRA:NM_001347828.2:exon5:c.C445A:p.P149T | | | |
| D034 | BM | CEBPA | CEBPA:NM_001287424.2:exon1:c.423dupT:p.D142*, | stopgain | 18.54 | 0.3076923 |
| | | | CEBPA:NM_001287435.1:exon1:c.276dupT:p.D93*, | | | |
| | | | CEBPA:NM_004364.5:exon1:c.318dupT:p.D107* | | | |
| | | CEBPA | CEBPA:NM_001285829.1:exon1:c.562_{—} | nonframeshift | 67.63 | 0.3809524 |
| | | | 563insCGGGCCGCA:p.R187_N188insTGR, | insertion | | |
| | | | CEBPA:NM_001287424.2:exon1:c.1024_{—} | | | |
| | | | 1025insCGGGCCGCA:p.R341_N342insTGR, CEBPA:NM_{—} | | | |
| | | | 001287435.1:exon1:c.877_878insCGGGCCGCA:p.R292_{—} | | | |
| | | | N293insTGR, CEBPA:NM_004364.5:exon1:c.919_{—} | | | |
| | | | 920insCGGGCCGCA:p.R306_N307insTGR | | | |
| | | EED | EED:NM_001330334.1:exon10:c.C1066T:p.R356C, | nonsynonymous | 170.13 | 0.5202703 |
| | | | EED:NM_003797.5:exon12:c.C1306T:p.R436C, | SNV | | |
| | | | EED:NM_001308007.1:exon13:c.C1381T:p.R461C | | | |
| | | WT1 | WT1:NM_000378.6:exon6:c.A1064G:p.D355G, | nonsynonymous | 15.27 | 0.4 |
| | | | WT1:NM_001198552.2:exon6:c.A413G:p.D138G, | SNV | | |
| | | | WT1:NM_001198551.1:exon7:c.A464G:p.D155G, | | | |
| | | | WT1:NM_024424.5:exon7:c.A1115G:p.D372G, | | | |
| | | | WT1:NM_024426.6:exon7:c.A1115G:p.D372G | | | |
| D035 | BM | DNMT3A | DNMT3A:NM_001320893.1:exon18:c.C2188T:p.R730C, | nonsynonymous | 200 | 0.4413681 |
| | | | DNMT3A:NM_001375819.1:exon18:c.C1975T:p.R659C, | SNV | | |
| | | | DNMT3A:NM_153759.3:exon19:c.C2077T:p.R693C, | | | |
| | | | DNMT3A:NM_022552.5:exon23:c.C2644T:p.R882C, | | | |
| | | | DNMT3A:NM_175629.2:exon23:c.C2644T:p.R882C | | | |
| | | IDH2 | IDH2:NM_001290114.2:exon2:c.G125A:p.R42K, | nonsynonymous | 200 | 0.4163701 |
| | | | IDH2:NM_001289910.1:exon4:c.G359A:p.R120K, | SNV | | |
| | | | IDH2:NM_002168.4:exon4:c.G515A:p.R172K | | | |
| D036 | BM | BCOR | BCOR:NM_001123384.2:exon6:c.3185_3189del:p.C1062Lfs*2, | frameshift | 200 | 0.5543478 |
| | | | BCOR:NM_001123383.1:exon7:c.3239_3243del:p.C1080Lfs*2, | deletion | | |
| | | | BCOR:NM_001123385.2:exon7:c.3239_3243del:p.C1080Lfs*2, | | | |
| | | | BCOR:NM_017745.6:exon7:c.3239_3243del:p.C1080Lfs*2 | | | |
| | | BCORL1 | BCORL1:NM_001379450.1:exon8:c.C4258T:p.R1420X, | stopgain | 200 | 0.5704918 |
| | | | BCORL1:NM_001379451.1:exon8:c.C4258T:p.R1420X, | | | |
| | | | BCORL1:NM_021946.5:exon8:c.C4258T:p.R1420X, | | | |
| | | | BCORL1:NM_001184772.3:exon9:c.C4258T:p.R1420X | | | |
| | | KRAS | KRAS:NM_001369786.1:exon2:c.G35A:p.G12D, | nonsynonymous | 131.79 | 0.1882353 |
| | | | KRAS:NM_001369787.1:exon2:c.G35A:p.G12D, | SNV | | |
| | | | KRAS:NM_004985.5:exon2:c.G35A:p.G12D, | | | |
| | | | KRAS:NM_033360.4:exon2:c.G35A:p.G12D | | | |
| | | RUNX1 | RUNX1:NM_001001890.3:exon2:c.T406G:p.F136V, | nonsynonymous | 200 | 0.3026316 |
| | | | RUNX1:NM_001122607.2:exon2:c.T406G:p.F136V, | SNV | | |
| | | | RUNX1:NM_001754.5:exon5:c.T487G:p.F163V | | | |
| | | RUNX1 | RUNX1:NM_001001890.3:exon3:c.G521A:p.R174Q, | nonsynonymous | 200 | 0.3324808 |
| | | | RUNX1:NM_001122607.2:exon3:c.G521A:p.R174Q, | SNV | | |
| | | | RUNX1:NM_001754.5:exon6:c.G602A:p.R201Q | | | |
| D037 | BM | PMS2 | PMS2:NM_000535.7:exon4:c.T343C:p.C115R, | nonsynonymous | 200 | 0.4606299 |
| | | | PMS2:NM_001322006.2:exon4:c.T343C:p.C115R, | SNV | | |
| | | | PMS2:NM_001322014.2:exon4:c.T343C:p.C115R | | | |
| | | SUZ12 | SUZ12:NM_001321207.2:exon12:c.C1424T:p.S475F, | nonsynonymous | 200 | 0.4786096 |
| | | | SUZ12:NM_015355.4:exon13:c.C1493T:p.S498F | SNV | | |
| | | TET2 | TET2:NM_001127208.3:exon6:c.T3633A:p.C1211X | stopgain | 200 | 0.4825581 |
| D038 | BM | FLT3 | FLT3:NM_004119.3:exon14:c.1826_{—} | nonframeshift | 44.98 | 0.2674419 |
| | | | 1827insATGGGAGTTTCCAAGAGAAAA:p.E608_{—} | insertion | | |
| | | | N609insKWEFPRE | | | |
| | | IDH2 | IDH2:NM_001290114.2:exon2:c.G29A:p.R10Q, | nonsynonymous | 160.61 | 0.483871 |
| | | | IDH2:NM_001289910.1:exon4:c.G263A:p.R88Q, | SNV | | |
| | | | IDH2:NM_002168.4:exon4:c.G419A:p.R140Q | | | |
| | | NPM1 | NPM1:NM_001355010.1:exon7:c.478_479insTCTG:p.W161Cfs*12, | frameshift | 61.89 | 0.4590164 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D039 | BM | KRAS | KRAS:NM_001369786.1:exon3:c.A183C:p.Q61H, | nonsynonymous | 200 | 0.3677758 |
| | | | KRAS:NM_001369787.1:exon3:c.A183C:p.Q61H, | SNV | | |
| | | | KRAS:NM_004985.5:exon3:c.A183C:p.Q61H, | | | |
| | | | KRAS:NM_033360.4:exon3:c.A183C:p.Q61H | | | |
| D040 | BM | DNMT3A | DNMT3A:NM_001320893.1:exon17:c.C2089T:p.P697S, | nonsynonymous | 200 | 0.9815385 |
| | | | DNMT3A:NM_001375819.1:exon17:c.C1876T:p.P626S, | SNV | | |
| | | | DNMT3A:NM_153759.3:exon18:c.C1978T:p.P660S, | | | |
| | | | DNMT3A:NM_022552.5:exon22:c.C2545T:p.P849S, | | | |
| | | | DNMT3A:NM_175629.2:exon22:c.C2545T:p.P849S | | | |
| | | ZRSR2 | ZRSR2:NM_005089.4:exon11:c.C1228T:p.R410C | nonsynonymous | 200 | 0.5617978 |
| | | | | SNV | | |
| D041 | BM | DNMT3A | DNMT3A:NM_001320893.1:exon18:c.C2188T:p.R730C, | nonsynonymous | 200 | 0.4375 |
| | | | DNMT3A:NM_001375819.1:exon18:c.C1975T:p.R659C, | SNV | | |
| | | | DNMT3A:NM_153759.3:exon19:c.C2077T:p.R693C, | | | |
| | | | DNMT3A:NM_022552.5:exon23:c.C2644T:p.R882C, | | | |
| | | | DNMT3A:NM_175629.2:exon23:c.C2644T:p.R882C | | | |
| D042 | BM | BCOR | NM_001123383.1:exon12:c.4639 + 1G > A; | splice site | 58.61 | 0.4642857 |
| | | | NM_001123385.2:exon12:c.4741 + 1G > A; | mutation | | |
| | | | NM_001123384.2:exon11:c.4585 + 1G > A; | | | |
| | | | NM_017745.6:exon12:c.4639 + 1G > A | | | |
| | | KMT2A | KMT2A:NM_001197104.2:exon2:c.G449C:p.G150A, | nonsynonymous | 111.7 | 0.3934426 |
| | | | KMT2A:NM_005933.4:exon2:c.G449C:p.G150A | SNV | | |
| | | NPM1 | NPM1:NM_001355010.1:exon7:c.478_479insTCTG:p.W161Cfs*12, | frameshift | 24.73 | 0.3235294 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D043 | PB | IDH2 | IDH2:NM_001290114.2:exon2:c.G29A:p.R10Q, | nonsynonymous | 200 | 0.4738806 |
| | | | IDH2:NM_001289910.1:exon4:c.G263A:p.R88Q, | SNV | | |
| | | | IDH2:NM_002168.4:exon4:c.G419A:p.R140Q | | | |
| | | NPM1 | NPM1:NM_001355010.1:exon7:c.478_479insTCTG:p.W161Cfs*12, | frameshift | 69.33 | 0.3181818 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D044 | BM | DNMT3A | DNMT3A:NM_001320893.1:exon18:c.C2188T:p.R730C, | nonsynonymous | 200 | 0.4451613 |
| | | | DNMT3A:NM_001375819.1:exon18:c.C1975T:p.R659C, | SNV | | |
| | | | DNMT3A:NM_153759.3:exon19:c.C2077T:p.R693C, | | | |
| | | | DNMT3A:NM_022552.5:exon23:c.C2644T:p.R882C, | | | |
| | | | DNMT3A:NM_175629.2:exon23:c.C2644T:p.R882C | | | |
| | | RUNX1 | RUNX1:NM_001001890.3:exon3:c.G521A:p.R174Q, | nonsynonymous | 200 | 0.5240642 |
| | | | RUNX1:NM_001122607.2:exon3:c.G521A:p.R174Q, | SNV | | |
| | | | RUNX1:NM_001754.5:exon6:c.G602A:p.R201Q | | | |
| | | U2AF1; | U2AF1:NM_001025203.1:exon2:c.C101T:p.S34F, | nonsynonymous | 156.94 | 0.5948276 |
| | | U2AF1L5 | U2AF1L5:NM_001320646.2:exon2:c.C101T:p.S34F, | SNV | | |
| | | | U2AF1L5:NM_001320648.2:exon2:c.C101T:p.S34F, | | | |
| | | | U2AF1L5:NM_001320650.2:exon2:c.C16T:p.L6F, | | | |
| | | | U2AF1:NM_006758.3:exon2:c.C101T:p.S34F | | | |
| D045 | BM | CBLC | CBLC:NM_001130852.1:exon4:c.G749A:p.R250H, | nonsynonymous | 200 | 0.3646724 |
| | | | CBLC:NM_012116.4:exon4:c.G749A:pR250H | SNV | | |
| | | NRAS | NRAS:NM_002524.5:exon3:c.A182G:p.Q61R | nonsynonymous | 19.26 | 0.1823529 |
| | | | | SNV | | |
| | | NRAS | NRAS:NM_002524.5:exon3:c.C181A:p.Q61K | nonsynonymous | 44.63 | 0.0996885 |
| | | | | SNV | | |
| D046 | BM | SMC1A | SMC1A:NM_006306.4:exon22:c.G3391A:p.G1131R, | nonsynonymous | 200 | 0.4917127 |
| | | | SMC1A:NM_001281463.1:exon23:c.G3325A:p.G1109R | SNV | | |
| D047 | PB | KIT | KIT:NM_000222.3:exon17:c.A2447T:p.D816V, | nonsynonymous | 33.18 | 0.0872727 |
| | | | KIT:NM_001093772.2:exon17:c.A2435T:p.D812V, | SNV | | |
| | | | KIT:NM_001385284.1:exon17:c.A2450T:p.D817V, | | | |
| | | | KIT:NM_001385285.1:exon17:c.A2444T:p.D815V, | | | |
| | | | KIT:NM_001385286.1:exon17:c.A2432T:p.D811V, | | | |
| | | | KIT:NM_001385288.1:exon17:c.A2438T:p.D813V, | | | |
| | | | KIT:NM_001385290.1:exon17:c.A2447T:p.D816V, | | | |
| | | | KIT:NM_001385292.1:exon17:c.A2435T:p.D812V | | | |
| | | TET2 | TET2:NM_001127208.3:exon3:c.822delC:p.N275Ifs*18, | frameshift | 175.61 | 0.3647541 |
| | | | TET2:NM_017628.4:exon3:c.822delC:p.N275Ifs*18 | deletion | | |
| D048 | BM | IDH1 | IDH1:NM_001282386.1:exon4:c.C394A:p.R132S, | nonsynonymous | 200 | 0.4412811 |
| | | | IDH1:NM_001282387.1:exon4:c.C394A:p.R132S, | SNV | | |
| | | | IDH1:NM_005896.4:exon4:c.C394A:p.R132S | | | |
| | | NPM1 | NPM1:NM_001355010.1:exon7:c.480_481insTGCT:p.W161Cfs*12, | frameshift | 146.2 | 0.4857143 |
| | | | NPM1:NM_001355007.1:exon10:c.669_670insTGCT:p.W224Cfs*12, | insertion | | |
| | | | NPM1:NM_199185.3:exon10:c.774_775insTGCT:p.W259Cfs*12, | | | |
| | | | NPM1:NM_002520.7:exon11:c.861_862insTGCT:p.W288Cfs*12, | | | |
| | | | NPM1:NM_001355006.1:exon12:c.861_862insTGCT:p.W288Cfs*12 | | | |
| D049 | BM | CBL | CBL:NM_005188.4:exon16:c.A2708G:p.H903R | nonsynonymous | 200 | 0.4900398 |
| | | | | SNV | | |
| | | IDH2 | IDH2:NM_001290114.2:exon2:c.G29A:p.R10Q, | nonsynonymous | 164.22 | 0.4364641 |
| | | | IDH2:NM_001289910.1:exon4:c.G263A:p.R88Q, | SNV | | |
| | | | IDH2:NM_002168.4:exon4:c.G419A:p.R140Q | | | |
| | | STAG2 | STAG2:NM_001375375.1:exon13:c.1218_1227del:p.A407Vfs*15, | frameshift | 200 | 0.5113636 |
| | | | STAG2:NM_006603.5:exon13:c.1218_1227del:p.A407Vfs*15, | deletion | | |
| | | | STAG2:NM_001042749.2:exon14:c.1218_1227del:p.A407Vfs*15, | | | |
| | | | STAG2:NM_001042750.2:exon14:c.1218_1227del:p.A407Vfs*15, | | | |
| | | | STAG2:NM_001042751.2:exon14:c.1218_1227del:p.A407Vfs*15, | | | |
| | | | STAG2:NM_001282418.2:exon14:c.1218_1227del:p.A407Vfs*15 | | | |
| D050 | BM | BCORL1 | BCORL1:NM_021946.5:exon13:c.5058_5059del:p.V1687Gfs*57, | frameshift | 200 | 0.8969072 |
| | | | BCORL1:NM_001379450.1:exon14:c.5280_5281del:p.V1761Gfs*57, | deletion | | |
| | | | BCORL1:NM_001379451.1:exon14:c.5280_5281del:p.V1761Gfs*57, | | | |
| | | | BCORL1:NM_001184772.3:exon15:c.5280_5281del:p.V1761Gfs*57 | | | |
| | | CEBPA | CEBPA:NM_001287424.2:exon1:c.330_331insCC:p.E111Pfs*85, | frameshift | 160.28 | 0.4375 |
| | | | CEBPA:NM_001287435.1:exon1:c.183_184insCC:p.E62Pfs*85, | insertion | | |
| | | | CEBPA:NM_004364.5:exon1:c.225_226insCC:p.E76Pfs*85 | | | |
| | | DNMT3A | DNMT3A:NM_001320893.1:exon18:c.G2189A:p.R730H, | nonsynonymous | 200 | 0.9402697 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| D051 | BM | DNMT3A | DNMT3A:NM_001320893.1:exon18:c.G2189A:p.R730H, | nonsynonymous | 200 | 0.457265 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | NPM1 | NPM1:NM_001355010.1:exon7:c.482_483insTAGA:p.W161Cfs*12, | frameshift | 62.48 | 0.5192308 |
| | | | NPM1:NM_001355007.1:exon10:c.671_672insTAGA:p.W224Cfs*12, | insertion | | |
| | | | NPM1:NM_199185.3:exon10:c.776_777insTAGA:p.W259Cfs*12, | | | |
| | | | NPM1:NM_002520.7:exon11:c.863_864insTAGA:p.W288Cfs*12, | | | |
| | | | NPM1:NM_001355006.1:exon12:c.863_864insTAGA:p.W288Cfs*12 | | | |
| | | TET2 | TET2:NM_001127208.3:exon6:c.C3640T:p.R1214W | nonsynonymous | 184.39 | 0.4913295 |
| | | | | SNV | | |
| | | WRN | WRN:NM_000553.6:exon19:c.C2108G:p.T703S | nonsynonymous | 114.49 | 0.4016393 |
| | | | | SNV | | |
| D052 | BM | CSF1R | CSF1R:NM_001288705.3:exon11:c.1647_1655delinsAAC:p.W550_{—} | nonframeshift | 24.36 | 0.1284404 |
| | | | 1552delinsT, CSF1R:NM_001375321.1:exon11:c.1203_{—} | substitution | | |
| | | | 1211delinsAAC:p.W402_1404delinsT, CSF1R:NM_{—} | | | |
| | | | 005211.3:exon12:c.1647_1655delinsAAC:p.W550_I552delinsT, | | | |
| | | | CSF1R:NM_001349736.1:exon13:c.1647_1655delinsAAC:p.W550_{—} | | | |
| | | | 1552delinsT, CSF1R:NM_001375320.1:exon13:c.1647_{—} | | | |
| | | | 1655delinsAAC:p.W550_1552delinsT | | | |
| | | IKZF1 | IKZF1:NM_001220767.2:exon3:c.A215G:p.N72S, | nonsynonymous | 70.16 | 0.3928571 |
| | | | IKZF1:NM_001220770.2:exon3:c.A215G:p.N72S, | SNV | | |
| | | | IKZF1:NM_001220768.2:exon4:c.A476G:p.N159S, | | | |
| | | | IKZF1:NM_001291838.2:exon4:c.A215G:p.N72S, | | | |
| | | | IKZF1:NM_001291839.2:exon4:c.A215G:p.N72S, | | | |
| | | | IKZF1:NM_001220765.3:exon5:c.A476G:p.N159S, | | | |
| | | | IKZF1:NM_001291837.2:exon5:c.A476G:p.N159S, | | | |
| | | | IKZF1:NM_006060.6:exon5:c.A476G:p.N159S | | | |
| | | RUNX1 | RUNX1:NM_001001890.3:exon3:c.G521A:p.R174Q, | nonsynonymous | 170.43 | 0.6293103 |
| | | | RUNX1:NM_001122607.2:exon3:c.G521A:p.R174Q, | SNV | | |
| | | | RUNX1:NM_001754.5:exon6:c.G602A:p.R201Q | | | |
| | | SETBP1 | SETBP1:NM_001379141.1:exon6:c.A4187T:p.K1396M, | nonsynonymous | 97.87 | 0.4705882 |
| | | | SETBP1:NM_001379142.1:exon6:c.A4187T:p.K1396M, | SNV | | |
| | | | SETBP1:NM_015559.3:exon6:c.A4187T:p.K1396M | | | |
| | | SRSF2 | SRSF2:NM_001195427.2:exon1:c.C284G:p.P95R, | nonsynonymous | 34.89 | 0.2133333 |
| | | | SRSF2:NM_003016.4:exon1:c.C284G:p.P95R | SNV | | |
| D053 | BM | DNMT3A | DNMT3A:NM_001320893.1:exon9:c.G1171T:p.G391C, | nonsynonymous | 200 | 0.4772727 |
| | | | DNMT3A:NM_001375819.1:exon9:c.G958T:p.G320C, | SNV | | |
| | | | DNMT3A:NM_153759.3:exon10:c.G1060T:p.G354C, | | | |
| | | | DNMT3A:NM_022552.5:exon14:c.G1627T:p.G543C, | | | |
| | | | DNMT3A:NM_175629.2:exon14:c.G1627T:p.G543C | | | |
| | | FLT3 | FLT3:NM_004119.3:exon20:c.2508_2510del:p.I836del | nonframeshift | 36.38 | 0.094697 |
| | | | | deletion | | |
| | | KAT6A | KAT6A:NM_006766.5:exon17:c.G3937A:p.D1313N | nonsynonymous | 200 | 0.5178998 |
| | | | | SNV | | |
| | | NPM1 | NPM1:NM_001355010.1:exon7:c.478_479insTCTG:p.W161Cfs*12, | frameshift | 90.17 | 0.4134615 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D054 | BM | ASXL1 | ASXL1:NM_001363734.1:exon11:c.C1603T:p.R535W, | nonsynonymous | 200 | 0.4945848 |
| | | | ASXL1:NM_015338.6:exon12:c.C1786T:p.R596W | SNV | | |
| | | DNMT3A | DNMT3A:NM_001320893.1:exon14:c.1854dupG:p.R619Afs*11, | frameshift | 151.09 | 0.3865979 |
| | | | DNMT3A:NM_001375819.1:exon14:c.1641dupG:p.R548Afs*11, | insertion | | |
| | | | DNMT3A:NM_153759.3:exon15:c.1743dupG:p.R582Afs*11, | | | |
| | | | DNMT3A:NM_022552.5:exon19:c.2310dupG:p.R771Afs*11, | | | |
| | | | DNMT3A:NM_175629.2:exon19:c.2310dupG:p.R771Afs*11 | | | |
| | | IDH2 | IDH2:NM_001290114.2:exon2:c.G125A:p.R42K, | nonsynonymous | 200 | 0.4021352 |
| | | | IDH2:NM_001289910.1:exon4:c.G359A:p.R120K, | SNV | | |
| | | | IDH2:NM_002168.4:exon4:c.G515A:p.R172K | | | |
| D055 | BM | CEBPA | CEBPA:NM_001285829.1:exon1:c.589_590insTGG:p.L196_{—} | nonframeshift | 74.5 | 0.3789474 |
| | | | E197insV, CEBPA:NM_001287424.2:exon1:c.1051_{—} | insertion | | |
| | | | 1052insTGG:p.L350_E351insV, CEBPA:NM_{—} | | | |
| | | | 001287435.1:exon1:c.904_905insTGG:p.L301_E302insV, | | | |
| | | | CEBPA:NM_004364.5:exon1:c.946_947insTGG:p.L315_E316insV | | | |
| | | CEBPA | CEBPA:NM_001287424.2:exon1:c.222delC:p.A75Rfs*120, | frameshift | 165.33 | 0.4278075 |
| | | | CEBPA:NM_001287435.1:exon1:c.75delC:p.A26Rfs*120, | deletion | | |
| | | | CEBPA:NM_004364.5:exon1:c.117delC:p.A40Rfs*120 | | | |
| | | KMT2A | KMT2A:NM_001197104.2:exon27:c.10698_10700del:p.S3568del, | nonframeshift | 183.12 | 0.4804469 |
| | | | KMT2A:NM_005933.4:exon27:c.10689_10691del:p.S3565del | deletion | | |
| | | WT1 | WT1:NM_000378.6:exon6:c.1091_1092insCC:p.T365Rfs*73, | frameshift | 128.55 | 0.4452555 |
| | | | WT1:NM_001198552.2:exon6:c.440_441insCC:p.T148Rfs*73, | insertion | | |
| | | | WT1:NM_001198551.1:exon7:c.491_492insCC:p.T165Rfs*73, | | | |
| | | | WT1:NM_024424.5:exon7:c.1142_1143insCC:p.T382Rfs*73, | | | |
| | | | WT1:NM_024426.6:exon7:c.1142_1143insCC:p.T382Rfs*73 | | | |
| | | WT1 | WT1:NM_001367854.1:exon5:c.G217A:p.D73N, | nonsynonymous | 200 | 0.5431755 |
| | | | WT1:NM_000378.6:exon8:c.G1354A:p.D452N, | SNV | | |
| | | | WT1:NM_001198552.2:exon8:c.G703A:p.D235N, | | | |
| | | | WT1:NM_001198551.1:exon9:c.G754A:p.D252N, | | | |
| | | | WT1:NM_024424.5:exon9:c.G1405A:p.D469N, | | | |
| | | | WT1:NM_024426.6:exon9:c.G1405A:p.D469N | | | |
| D057 | BM | MYC | MYC:NM_001354870.1:exon2:c.C218T:p.P73L, | nonsynonymous | 101.26 | 0.1859756 |
| | | | MYC:NM_002467.6:exon2:c.C221T:p.P74L | SNV | | |
| D058 | BM | DNMT3A | DNMT3A:NM_001320893.1:exon12:c.G1520A:p.R507H, | nonsynonymous | 200 | 0.4383886 |
| | | | DNMT3A:NM_001375819.1:exon12:c.G1307A:p.R436H, | SNV | | |
| | | | DNMT3A:NM_153759.3:exon13:c.G1409A:p.R470H, | | | |
| | | | DNMT3A:NM_022552.5:exon17:c.G1976A:p.R659H, | | | |
| | | | DNMT3A:NM_175629.2:exon17:c.G1976A:p.R659H | | | |
| | | NPM1 | NPM1:NM_001355010.1:exon7:c.480_481insTGCA:p.W161Cfs*12, | frameshift | 200 | 0.4513514 |
| | | | NPM1:NM_001355007.1:exon10:c.669_670insTGCA:p.W224Cfs*12, | insertion | | |
| | | | NPM1:NM_199185.3:exon10:c.774_775insTGCA:p.W259Cfs*12, | | | |
| | | | NPM1:NM_002520.7:exon11:c.861_862insTGCA:p.W288Cfs*12, | | | |
| | | | NPM1:NM_001355006.1:exon12:c.861_862insTGCA:p.W288Cfs*12 | | | |
| | | RAD21 | RAD21:NM_006265.3:exon2:c.103_112del:p.C35Rfs*12 | frameshift | 187.89 | 0.5054348 |
| | | | | deletion | | |
| | | SRP72 | SRP72:NM_001267722.2:exon8:c.G788T:p.R263L, | nonsynonymous | 200 | 0.5328467 |
| | | | SRP72:NM_006947.4:exon10:c.G971T:p.R324L | SNV | | |
| D059 | BM | CEBPA | CEBPA:NM_001285829.1:exon1:c.563_{—} | nonframeshift | 104.66 | 0.3661972 |
| | | | 564insGGCCAAGCAGCGCAA:p.R187_N188insKAKQR, | insertion | | |
| | | | CEBPA:NM_001287424.2:exon1:c.1025_{—} | | | |
| | | | 1026insGGCCAAGCAGCGCAA:p.R341_N342insKAKQR, | | | |
| | | | CEBPA:NM_001287435.1:exon1:c.878_{—} | | | |
| | | | 879insGGCCAAGCAGCGCAA:p.R292_{—} | | | |
| | | | N293insKAKQR, CEBPA:NM_004364.5:exon1:c.920_{—} | | | |
| | | | 921insGGCCAAGCAGCGCAA:p.R306_N307insKAKQR | | | |
| | | CEBPA | CEBPA:NM_001287424.2:exon1:c.173dupC:p.H59Afs*84, | frameshift | 143.84 | 0.373057 |
| | | | CEBPA:NM_001287435.1:exon1:c.26dupC:p.H10Afs*84, | insertion | | |
| | | | CEBPA:NM_004364.5:exon1:c.68dupC:p.H24Afs*84 | | | |
| | | GATA2 | GATA2:NM_001145662.1:exon4:c.C910G:p.P304A, | nonsynonymous | 25.99 | 0.0756972 |
| | | | GATA2:NM_032638.5:exon4:c.C910G:p.P304A, | SNV | | |
| | | | GATA2:NM_001145661.2:exon5:c.C910G:p.P304A | | | |
| | | JAK3 | JAK3:NM_000215.4:exon15:c.C1969T:p.R657W | nonsynonymous | 38.77 | 0.0592593 |
| | | | | SNV | | |
| | | KDM6A | KDM6A:NM_001291415.2:exon9:c.710_711del:p.S238Cfs*24, | frameshift | 43.46 | 0.0703934 |
| | | | KDM6A:NM_001291416.1:exon9:c.710_711del:p.S238Cfs*24, | deletion | | |
| | | | KDM6A:NM_001291417.1:exon9:c.710_711del:p.S238Cfs*24, | | | |
| | | | KDM6A:NM_001291418.1:exon9:c.710_711del:p.S238Cfs*24, | | | |
| | | | KDM6A:NM_021140.3:exon9:c.710_711del:p.S238Cfs*24 | | | |
| | | WT1 | WT1:NM_000378.6:exon6:c.1108_1109insTCGG:p.A370Vfs*4, | frameshift | 22.79 | 0.0727273 |
| | | | WT1:NM_001198552.2:exon6:c.457_458insTCGG:p.A153Vfs*4, | insertion | | |
| | | | WT1:NM_001198551.1:exon7:c.508_509insTCGG:p.A170Vfs*4, | | | |
| | | | WT1:NM_024424.5:exon7:c.1159_1160insTCGG:p.A387Vfs*4, | | | |
| | | | WT1:NM_024426.6:exon7:c.1159_1160insTCGG:p.A387Vfs*4 | | | |
| D060 | BM | CSF3R | CSF3R:NM_000760.4:exon17:c.C2221T:p.Q741X, | stopgain | 92.93 | 0.375 |
| | | | CSF3R:NM_156039.3:exon17:c.C2302T:p.Q768X, | | | |
| | | | CSF3R:NM_172313.3:exon17:c.C2221T:p.Q741X | | | |
| | | DNMT3A | DNMT3A:NM_001320893.1:exon17:c.C2090A:p.P697H, | nonsynonymous | 82.01 | 0.3804348 |
| | | | DNMT3A:NM_001375819.1:exon17:c.C1877A:p.P626H, | SNV | | |
| | | | DNMT3A:NM_153759.3:exon18:c.C1979A:p.P660H, | | | |
| | | | DNMT3A:NM_022552.5:exon22:c.C2546A:p.P849H, | | | |
| | | | DNMT3A:NM_175629.2:exon22:c.C2546A:p.P849H | | | |
| D061 | BM | PAX5 | PAX5:NM_001280551.2:exon7:c.C628A:p.P210T, | nonsynonymous | 124.55 | 0.5263158 |
| | | | PAX5:NM_001280552.2:exon8:c.C952A:p.P318T, | SNV | | |
| | | | PAX5:NM_001280553.2:exon8:c.C925A:p.P309T, | | | |
| | | | PAX5:NM_001280555.2:exon8:c.C841A:p.P281T, | | | |
| | | | PAX5:NM_001280547.2:exon9:c.C1039A:p.P347T, | | | |
| | | | PAX5:NM_001280548.2:exon9:c.C1054A:p.P352T, | | | |
| | | | PAX5:NM_001280554.2:exon9:c.C1012A:p.P338T, | | | |
| | | | PAX5:NM_001280556.2:exon9:c.C817A:p.P273T, | | | |
| | | | PAX5:NM_016734.3:exon10:c.C1141A:p.P381T | | | |
| | | STAG2 | STAG2:NM_001375375.1:exon29:c.C3224A:p.S1075X, | stopgain | 189.39 | 0.9285714 |
| | | | STAG2:NM_006603.5:exon29:c.C3224A:p.S1075X, | | | |
| | | | STAG2:NM_001042749.2:exon30:c.C3224A:p.S1075X, | | | |
| | | | STAG2:NM_001042750.2:exon30:c.C3224A:p.S1075X, | | | |
| | | | STAG2:NM_001042751.2:exon30:c.C3224A:p.S1075X, | | | |
| | | | STAG2:NM_001282418.2:exon30:c.C3224A:p.S1075X | | | |
| D062 | BM | ASXL1 | ASXL1:NM_001363734.1:exon11:c.C1684T:p.Q562X, | stopgain | 28.49 | 0.0505263 |
| | | | ASXL1:NM_015338.6:exon12:c.C1867T:p.Q623X | | | |
| | | CBL | CBL:NM_005188.4:exon8:c.T1111A:p.Y371N | nonsynonymous | 200 | 0.322314 |
| | | | | SNV | | |
| | | IDH2 | IDH2:NM_001290114.2:exon2:c.G29A:p.R10Q, | nonsynonymous | 200 | 0.4723926 |
| | | | IDH2:NM_001289910.1:exon4:c.G263A:p.R88Q, | SNV | | |
| | | | IDH2:NM_002168.4:exon4:c.G419A:p.R140Q | | | |
| | | NPM1 | NPM1:NM_001355010.1:exon7:c.478_{—} | frameshift | 124.65 | 0.4754098 |
| | | | 479insTCTG:p.W161Cfs*12, NPM1:NM_{—} | insertion | | |
| | | | 001355007.1:exon10:c.667_668insTCTG:p.W224Cfs*12, | | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | RUNX1 | RUNX1:NM_001001890.3:exon5:c.849_850insGCATG:p.T284Afs*2, | stopgain | 148.21 | 0.177264 |
| | | | RUNX1:NM_001754.5:exon8:c.930_931insGCATG:p.T311Afs*2 | | | |
| | | SRSF2 | SRSF2:NM_001195427.2:exon1:c.284_307de1:p.P95_R102del, | nonframeshift | 161.21 | 0.2776025 |
| | | | SRSF2:NM_003016.4:exon1:c.284_307del:p.P95_R102del | deletion | | |
| D063 | PB | CBL | CBL:NM_005188.4:exon9:c.T1246A:p.C416S | nonsynonymous | 200 | 0.8275862 |
| | | | | SNV | | |
| | | DNMT3A | DNMT3A:NM_001320893.1:exon11:c.1424delC:p.P475Qfs*24, | frameshift | 200 | 0.4155844 |
| | | | DNMT3A:NM_001375819.1:exon11:c.1211delC:p.P404Qfs*24, | deletion | | |
| | | | DNMT3A:NM_153759.3:exon12:c.1313delC:p.P438Qfs*24, | | | |
| | | | DNMT3A:NM_022552.5:exon16:c.188OdelC:p.P627Qfs*24, | | | |
| | | | DNMT3A:NM_175629.2:exon16:c.1880delC:p.P627Qfs*24 | | | |
| | | NPM1 | NPM1:NM_001355010.1:exon7:c.480_481insTGCA:p.W161Cfs*12, | frameshift | 47.81 | 0.4 |
| | | | NPM1:NM_001355007.1:exon10:c.669_670insTGCA:p.W224Cfs*12, | insertion | | |
| | | | NPM1:NM_199185.3:exon10:c.774_775insTGCA:p.W259Cfs*12, | | | |
| | | | NPM1:NM_002520.7:exon11:c.861_862insTGCA:p.W288Cfs*12, | | | |
| | | | NPM1:NM_001355006.1:exon12:c.861_862insTGCA:p.W288Cfs*12 | | | |
| D064 | BM | DNMT3A | DNMT3A:NM_001320893.1:exon18:c.G2189A:p.R730H, | nonsynonymous | 200 | 0.3890877 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| D065 | PB | CEBPA | CEBPA:NM_001285829.1:exon1:c.670delC:p.R224Afs*79, | frameshift | 200 | 0.4617564 |
| | | | CEBPA:NM_001287424.2:exon1:c.1132delC:p.R378Afs*79, | deletion | | |
| | | | CEBPA:NM_001287435.1:exon1:c.985delC:p.R329Afs*79, | | | |
| | | | CEBPA:NM_004364.5:exon1:c.1027delC:p.R343Afs*79 | | | |
| | | RUNX1 | RUNX1:NM_001001890.3:exon6:c.902_903insT:p.A302Sfs*271, | frameshift | 200 | 0.5253731 |
| | | | RUNX1:NM_001754.5:exon9:c.983_984insT:p.A329Sfs*271 | insertion | | |
| | | TET2 | TET2:NM_001127208.3:exon3:c.1859dupA:p.Y620*, | stopgain | 148.73 | 0.4666667 |
| | | | TET2:NM_017628.4:exon3:c.1859dupA:p.Y620* | | | |
| | | TET2 | TET2:NM_001127208.3:exon10:c.C4210T:p.R1404X | stopgain | 173.78 | 0.5128205 |
| | | ASXL1 | ASXL1:NM_001363734.1:exon11:c.2140delT:p.L714*, | stopgain | 200 | 0.5426009 |
| | | | ASXL1:NM_015338.6:exon12:c.2323delT:p.L775* | | | |
| | | PHF6 | PHF6:NM_001015877.2:exon9:c.900delT:p.Y301Tfs*50, | frameshift | 200 | 0.4381625 |
| | | | PHF6:NM_032458.3:exon9:c.900delT:p.Y301Tfs*50 | deletion | | |
| D066 | BM | ASXL2 | ASXL2:NM_001369346.1:exon4:c.C242G:p.S81C, | nonsynonymous | 152.27 | 0.4507042 |
| | | | ASXL2:NM_018263.6:exon5:c.C416G:p.S139C | SNV | | |
| | | CEBPA | CEBPA:NM_001287424.2:exon1:c.173_174insCC:p.H59Rfs*137, | frameshift | 27.15 | 0.1971831 |
| | | | CEBPA:NM_001287435.1:exon1:c.26_27insCC:p.H10Rfs*137, | insertion | | |
| | | | CEBPA:NM_004364.5:exon1:c.68_69insCC:p.H24Rfs*137 | | | |
| | | CEBPA | CEBPA:NM_001285829.1:exon1:c.568_573del:p.E190_T191del, | nonframeshift | 49.07 | 0.359375 |
| | | | CEBPA:NM_001287424.2:exon1:c.1030_1035del:p.E344_T345del, | deletion | | |
| | | | CEBPA:NM_001287435.1:exon1:c.883_888del:p.E295_T296del, | | | |
| | | | CEBPA:NM_004364.5:exon1:c.925_930del:p.E309_T310del | | | |
| | | GATA2 | GATA2:NM_001145662.1:exon4:c.G952A:p.A318T, | nonsynonymous | 137.63 | 0.4362416 |
| | | | GATA2:NM_032638.5:exon4:c.G952A:p.A318T, | SNV | | |
| | | | GATA2:NM_001145661.2:exon5:c.G952A:p.A318T | | | |
| | | NRAS | NRAS:NM_002524.5:exon2:c.G38A:p.G13D | nonsynonymous | 174.38 | 0.5197368 |
| | | | | SNV | | |
| | | TNFRSF13B | TNFRSF13B:NM_012452.3:exon5:c.853_854insGC:p.P285Rfs*40 | frameshift | 114.55 | 0.490566 |
| | | | | insertion | | |
| D067 | BM | CEBPA | CEBPA:NM_001287424.2:exon1:c.173dupC:p.H59Afs*84, | frameshift | 167.47 | 0.3766816 |
| | | | CEBPA:NM_001287435.1:exon1:c.26dupC:p.H10Afs*84, | insertion | | |
| | | | CEBPA:NM_004364.5:exon1:c.68dupC:p.H24Afs*84 | | | |
| | | EED | EED:NM_001308007.1:exon9:c.C937T:p.R313X, | stopgain | 200 | 0.3212435 |
| | | | EED:NM_003797.5:exon9:c.C937T:p.R313X | | | |
| | | EZH2 | EZH2:NM_001203249.2:exon16:c.C1787T:p.S596F, | nonsynonymous | 45.28 | 0.0941176 |
| | | | EZH2:NM_152998.3:exon16:c.C1823T:p.S608F, | SNV | | |
| | | | EZH2:NM_001203247.2:exon17:c.C1940T:p.S647F, | | | |
| | | | EZH2:NM_001203248.2:exon17:c.C1913T:p.S638F, | | | |
| | | | EZH2:NM_004456.5:exon17:c.C1955T:p.S652F | | | |
| | | KMT2A | KMT2A:NM_001197104.2:exon27:c.T9566C:p.13189T, | nonsynonymous | 200 | 0.5432099 |
| | | | KMT2A:NM_005933.4:exon27:c.T9557C:p.I3186T | SNV | | |
| D068 | BM | BCOR | BCOR:NM_001123384.2:exon12:c.4644_4645del:p.F1548Lfs*7, | frameshift | 79.3 | 0.3958333 |
| | | | BCOR:NM_001123383.1:exon13:c.4698_4699del:p.F1566Lfs*7, | deletion | | |
| | | | BCOR:NM_001123385.2:exon13:c.4800_4801del:p.F1600Lfs*7, | | | |
| | | | BCOR:NM_017745.6:exon13:c.4698_4699del:p.F1566Lfs*7 | | | |
| | | KRAS | KRAS:NM_001369786.1:exon2:c.G35T:p.G12V, | nonsynonymous | 200 | 0.7230047 |
| | | | KRAS:NM_001369787.1:exon2:c.G35T:p.G12V, | SNV | | |
| | | | KRAS:NM_004985.5:exon2:c.G35T:p.G12V, | | | |
| | | | KRAS:NM_033360.4:exon2:c.G35T:p.G12V | | | |
| | | SF3B1 | SF3B1:NM_012433.4:exon14:c.C1873T:p.R625C | nonsynonymous | 187.99 | 0.45 |
| | | | | SNV | | |
| D069 | BM | DNMT3A | DNMT3A:NM_001320893.1:exon17:c.A2069G:p.Q690R, | nonsynonymous | 73.79 | 0.5961538 |
| | | | DNMT3A:NM_001375819.1:exon17:c.A1856G:p.Q619R, | SNV | | |
| | | | DNMT3A:NM_153759.3:exon18:c.A1958G:p.Q653R, | | | |
| | | | DNMT3A:NM_022552.5:exon22:c.A2525G:p.Q842R, | | | |
| | | | DNMT3A:NM_175629.2:exon22:c.A2525G:p.Q842R | | | |
| | | FLT3 | FLT3:NM_004119.3:exon14:c.1782_{—} | nonframeshift | 16.97 | 0.0952381 |
| | | | 1783insGATAATGAGTACTTCTACGTTGATTTC:p.F594_{—} | insertion | | |
| | | | R595insDNEYFYVDF | | | |
| | | PTPN11 | PTPN11:NM_001330437.2:exon13:c.G1520A:p.G507E, | nonsynonymous | 15.06 | 0.1230769 |
| | | | PTPN11:NM_001374625.1:exon13:c.G1505A:p.G502E, | SNV | | |
| | | | PTPN11:NM_002834.5:exon13:c.G1508A:p.G503E | | | |
| | | STAG2 | STAG2:NM_001375375.1:exon13:c.G1279A:p.A427T, | nonsynonymous | 80.53 | 0.5964912 |
| | | | STAG2:NM_006603.5:exon13:c.G1279A:p.A427T, | SNV | | |
| | | | STAG2:NM_001042749.2:exon14:c.G1279A:p.A427T, | | | |
| | | | STAG2:NM_001042750.2:exon14:c.G1279A:p.A427T, | | | |
| | | | STAG2:NM_001042751.2:exon14:c.G1279A:p.A427T, | | | |
| | | | STAG2:NM_001282418.2:exon14:c.G1279A:p.A427T | | | |
| D070 | BM | NRAS | NRAS:NM_002524.5:exon3:c.C181A:p.Q61K | nonsynonymous | 103.39 | 0.3581081 |
| | | | | SNV | | |
| | | PTPN11 | PTPN11:NM_001330437.2:exon3:c.A227G:p.E76G, | nonsynonymous | 27.31 | 0.0621302 |
| | | | PTPN11:NM_001374625.1:exon3:c.A224G:p.E75G, | SNV | | |
| | | | PTPN11:NM_002834.5:exon3:c.A227G:p.E76G, | | | |
| | | | PTPN11:NM_080601.3:exon3:c.A227G:p.E76G | | | |
| D071 | BM | KDM6A | KDM6A:NM_001291418.1:exon21:c.C3194A:p.P1065Q, | nonsynonymous | 43.73 | 0.2923077 |
| | | | KDM6A:NM_001291421.1:exon21:c.C2543A:p.P848Q, | SNV | | |
| | | | KDM6A:NM_001291417.1:exon22:c.C3296A:p.P1099Q, | | | |
| | | | KDM6A:NM_001291416.1:exon23:c.C3452A:p.P1151Q, | | | |
| | | | KDM6A:NM_021140.3:exon23:c.C3431A:p.P1144Q, | | | |
| | | | KDM6A:NM_001291415.2:exon24:c.C3587A:p.P1196Q | | | |
| D072 | BM | SMC3 | SMC3:NM_005445.4:exon27:c.A3449G:p.D1150G | nonsynonymous | 155.46 | 0.4302326 |
| | | | | SNV | | |
| D073 | BM | NPM1 | NPM1:NM_001355010.1:exon7:c.480_481insTGCA:p.W161Cfs*12, | frameshift | 26.07 | 0.4074074 |
| | | | NPM1:NM_001355007.1:exon10:c.669_670insTGCA:p.W224Cfs*12, | insertion | | |
| | | | NPM1:NM_199185.3:exon10:c.774_775insTGCA:p.W259Cfs*12, | | | |
| | | | NPM1:NM_002520.7:exon11:c.861_862insTGCA:p.W288Cfs*12, | | | |
| | | | NPM1:NM_001355006.1:exon12:c.861_862insTGCA:p.W288Cfs*12 | | | |
| | | WT1 | WT1:NM_000378.6:exon6:c.1108_1109insTCGG:p.A370Vfs*4, | frameshift | 59.41 | 0.3733333 |
| | | | WT1:NM_001198552.2:exon6:c.457_458insTCGG:p.A153Vfs*4, | insertion | | |
| | | | WT1:NM_001198551.1:exon7:c.508_509insTCGG:p.A170Vfs*4, | | | |
| | | | WT1:NM_024424.5:exon7:c.1159_1160insTCGG:p.A387Vfs*4, | | | |
| | | | WT1:NM_024426.6:exon7:c.1159_1160insTCGG:p.A387Vfs*4 | | | |
| D074 | BM | RELN | RELN:NM_005045.4:exon45:c.T6938C:p.12313T, | nonsynonymous | 159.19 | 0.4932432 |
| | | | RELN:NM_173054.2:exon45:c.T6938C:p.I2313T | SNV | | |
| | | RUNX1 | RUNX1:NM_001001890.3:exon4:c.654dupC:p.T219Hfs*15, | frameshift | 39.27 | 0.36 |
| | | | RUNX1:NM_001122607.2:exon4:c.654dupC:p.T219Hfs*15, | insertion | | |
| | | | RUNX1:NM_001754.5:exon7:c.735dupC:p.T246Hfs*15 | | | |
| D075 | BM | DNMT3A | DNMT3A:NM_001320893.1:exon18:c.G2189A:p.R730H, | nonsynonymous | 200 | 0.4508929 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | NPM1 | NPM1:NM_001355010.1:exon7:c.478_{—} | frameshift | 43.71 | 0.3773585 |
| | | | 479insTCTG:p.W161Cfs*12, NPM1:NM_{—} | insertion | | |
| | | | 001355007.1:exon10:c.667_668insTCTG:p.W224Cfs*12, | | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D076 | BM | IDH1 | IDH1:NM_001282386.1:exon4:c.C394T:p.R132C, | nonsynonymous | 101.97 | 0.392 |
| | | | IDH1:NM_001282387.1:exon4:c.C394T:p.R132C, | SNV | | |
| | | | IDH1:NM_005896.4:exon4:c.C394T:p.R132C | | | |
| D077 | BM | FLT3 | FLT3:NM_004119.3:exon20:c.G2503T:p.D835Y | nonsynonymous | 48.85 | 0.2190476 |
| | | | | SNV | | |
| | | KDR | KDR:NM_002253.3:exon22:c.A3011T:p.H1004L | nonsynonymous | 55.27 | 0.2688172 |
| | | | | SNV | | |
| | | NF1 | NF1:NM_000267.3:exon9:c.T1058C:p.L353P, | nonsynonymous | 85.1 | 0.5441176 |
| | | | NF1:NM_001042492.3:exon9:c.T1058C:p.L353P, | SNV | | |
| | | | NF1:NM_001128147.3:exon9:c.T1058C:p.L353P | | | |
| | | NF1 | NF1:NM_000267.3:exon32:c.T4340A:p.V1447E, | nonsynonymous | 87.35 | 0.3978495 |
| | | | NF1:NM_001042492.3:exon33:c.T4403A:p.V1468E | SNV | | |
| | | TET2 | TET2:NM_001127208.3:exon10:c.C4240T:p.Q1414X | stopgain | 95.09 | 0.525 |
| | | TET2 | TET2:NM_001127208.3:exon3:c.C2725T:p.Q909X, | stopgain | 98.89 | 0.5584416 |
| | | | TET2:NM_017628.4:exon3:c.C2725T:p.Q909X | | | |
| D078 | BM | DNMT3A | DNMT3A:NM_001320893.1:exon12:c.G1594T:p.V532F, | nonsynonymous | 152.73 | 0.4596273 |
| | | | DNMT3A:NM_001375819.1:exon12:c.G1381T:p.V461F, | SNV | | |
| | | | DNMT3A:NM_153759.3:exon13:c.G1483T:p.V495F, | | | |
| | | | DNMT3A:NM_022552.5:exon17:c.G2050T:p.V684F, | | | |
| | | | DNMT3A:NM_175629.2:exon17:c.G2050T:p.V684F | | | |
| | | NPM1 | NPM1:NM_001355010.1:exon7:c.480_481insTGCA:p.W161Cfs*12, | frameshift | 59.27 | 0.442623 |
| | | | NPM1:NM_001355007.1:exon10:c.669_670insTGCA:p.W224Cfs*12, | insertion | | |
| | | | NPM1:NM_199185.3:exon10:c.774_775insTGCA:p.W259Cfs*12, | | | |
| | | | NPM1:NM_002520.7:exon11:c.861_862insTGCA:p.W288Cfs*12, | | | |
| | | | NPM1:NM_001355006.1:exon12:c.861_862insTGCA:p.W288Cfs*12 | | | |
| | | NRAS | NRAS:NM_002524.5:exon2:c.G38A:p.G13D | nonsynonymous | 17.62 | 0.0693642 |
| | | | | SNV | | |
| | | PAX5 | PAX5:NM_001280551.2:exon7:c.G640A:p.A214T, | nonsynonymous | 108.04 | 0.4126984 |
| | | | PAX5:NM_001280552.2:exon8:c.G964A:p.A322T, | SNV | | |
| | | | PAX5:NM_001280553.2:exon8:c.G937A:p.A313T, | | | |
| | | | PAX5:NM_001280555.2:exon8:c.G853A:p.A285T, | | | |
| | | | PAX5:NM_001280547.2:exon9:c.G1051A:p.A351T, | | | |
| | | | PAX5:NM_001280548.2:exon9:c.G1066A:p.A356T, | | | |
| | | | PAX5:NM_001280554.2:exon9:c.G1024A:p.A342T, | | | |
| | | | PAX5:NM_001280556.2:exon9:c.G829A:p.A277T, | | | |
| | | | PAX5:NM_016734.3:exon10:c.G1153A:p.A385T | | | |
| | | PTPN11 | PTPN11:NM_001330437.2:exon13:c.C1532A:p.T511K, | nonsynonymous | 35.75 | 0.1256831 |
| | | | PTPN11:NM_001374625.1:exon13:c.C1517A:p.T506K, | SNV | | |
| | | | PTPN11:NM_002834.5:exon13:c.C1520A:p.T507K | | | |
| D079 | BM | GATA2 | GATA2:NM_001145662.1:exon5:c.G1072A:p.A358T, | nonsynonymous | 200 | 0.4871795 |
| | | | GATA2:NM_032638.5:exon5:c.G1114A:p.A372T, | SNV | | |
| | | | GATA2:NM_001145661.2:exon6:c.G1114A:p.A372T | | | |
| | | KDM6A | KDM6A:NM_001291418.1:exon21:c.3100_3 | nonframeshift | 200 | 0.4326531 |
| | | | 101insGCTACA:p.V1034delinsGYI, | insertion | | |
| | | | KDM6A:NM_001291421.1:exon21:c.2449_{—} | | | |
| | | | 2450insGCTACA:p.V817delinsGYI, KDM6A:NM_{—} | | | |
| | | | 001291417.1:exon22:c.3202_{—} | | | |
| | | | 3203insGCTACA:p.V1068delinsGYI, KDM6A:NM_{—} | | | |
| | | | 001291416.1:exon23:c.3358_3359insGCTACA:p.V1120delinsGYI, | | | |
| | | | KDM6A:NM_021140.3:exon23:c.3337_{—} | | | |
| | | | 3338insGCTACA:p.V1113delinsGYI, KDM6A:NM_{—} | | | |
| | | | 001291415.2:exon24:c.3493_3494insGCTACA:p.V1165delinsGYI | | | |
| | | NOTCH1 | NOTCH1:NM_017617.5:exon25:c.T4420G:p.W1474G | nonsynonymous | 26.79 | 0.0575916 |
| | | | | SNV | | |
| | | NPM1 | NPM1:NM_001355010.1:exon7:c.478_{—} | frameshift | 75.39 | 0.3592233 |
| | | | 479insTCTG:p.W161Cfs*12, NPM1:NM_{—} | insertion | | |
| | | | 001355007.1:exon10:c.667_668insTCTG:p.W224Cfs*12, | | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | NRAS | NRAS:NM_002524.5:exon2:c.G35A:p.G12D | nonsynonymous | 200 | 0.4923077 |
| | | | | SNV | | |
| D080 | BM | CDKN2A | CDKN2A:NM_058197.5:exon1:c.254delA:p.K85Rfs*44 | frameshift | 54.76 | 0.4098361 |
| | | | | deletion | | |
| | | IDH2 | IDH2:NM_001290114.2:exon2:c.G29A:p.R10Q, | nonsynonymous | 51.18 | 0.3636364 |
| | | | IDH2:NM_001289910.1:exon4:c.G263A:p.R88Q, | SNV | | |
| | | | IDH2:NM_002168.4:exon4:c.G419A:p.R140Q | | | |
| | | KMT2C | KMT2C:NM_170606.3:exon49:c.C12433T:p.R4145C | nonsynonymous | 93.88 | 0.4230769 |
| | | | | SNV | | |
| | | NPM1 | NPM1:NM_001355010.1:exon7:c.478_{—} | frameshift | 69.15 | 0.6666667 |
| | | | 479insTCTG:p.W161Cfs*12, NPM1:NM_{—} | insertion | | |
| | | | 001355007.1:exon10:c.667_668insTCTG:p.W224Cfs*12, | | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | SUZ12 | SUZ12:NM_001321207.2:exon12:c.G1444C:p.D482H, | nonsynonymous | 127.31 | 0.4568966 |
| | | | SUZ12:NM_015355.4:exon13:c.G1513C:p.D505H | SNV | | |
| | | TET2 | TET2:NM_001127208.3:exon4:c.G3492A:p.M1164I | nonsynonymous | 96.08 | 0.3931624 |
| | | | | SNV | | |
| D081 | BM | CEBPA | CEBPA:NM_001285829.1:exon1:c.571_572insAGA:p.E190_{—} | nonframeshift | 96.14 | 0.4631579 |
| | | | T191insK, CEBPA:NM_001287424.2:exon1:c.1033_{—} | insertion | | |
| | | | 1034insAGA:p.E344T345insK, CEBPA:NM_{—} | | | |
| | | | 001287435.1:exon1:c.886_887insAGA:p.E295_T296insK, | | | |
| | | | CEBPA:NM_004364.5:exon1:c.928_929insAGA:p.E309_T310insK | | | |
| | | CEBPA | CEBPA:NM_001285829.1:exon1:c.670delC:p.R224Afs*79, | frameshift | 124.82 | 0.4566929 |
| | | | CEBPA:NM_001287424.2:exon1:c.1132delC:p.R378Afs*79, | deletion | | |
| | | | CEBPA:NM_001287435.1:exon1:c.985delC:p.R329Afs*79, | | | |
| | | | CEBPA:NM_004364.5:exon1:c.1027delC:p.R343Afs*79 | | | |
| | | GATA2 | GATA2:NM_001145662.1:exon4:c.A950T:p.N317I, | nonsynonymous | 196.2 | 0.5228758 |
| | | | GATA2:NM_032638.5:exon4:c.A950T:p.N317I, | SNV | | |
| | | | GATA2:NM_001145661.2:exon5:c.A950T:p.N317I | | | |
| D082 | BM | ASXL1 | ASXL1:NM_001363734.1:exon11:c.1705_1727del:p.E574Rfs*15, | frameshift | 37.96 | 0.2567568 |
| | | | ASXL1:NM_015338.6:exon12:c.1888_1910del:p.E635Rfs*15 | deletion | | |
| | | FLT3 | FLT3:NM_004119.3:exon20:c.G2503T:p.D835Y | nonsynonymous | 85.5 | 0.3627451 |
| | | | | SNV | | |
| | | NPAT | NPAT:NM_001321307.1:exon13:c.C1328T:p.T443I, | nonsynonymous | 171.75 | 0.5384615 |
| | | | NPAT:NM_002519.3:exon13:c.C1328T:p.T443I | SNV | | |
| | | PDGFRA | PDGFRA:NM_001347827.2:exon3:c.C275T:p.A92V, | nonsynonymous | 96.77 | 0.56 |
| | | | PDGFRA:NM_001347829.2:exon3:c.C275T:p.A92V, | SNV | | |
| | | | PDGFRA:NM_001347830.1:exon3:c.C314T:p.A105V, | | | |
| | | | PDGFRA:NM_006206.6:exon3:c.C275T:p.A92V, | | | |
| | | | PDGFRA:NM_001347828.2:exon4:c.C350T:p.A117V | | | |
| D083 | BM | NRAS | NRAS:NM_002524.5:exon2:c.G35A:p.G12D | nonsynonymous | 58.94 | 0.1585903 |
| | | | | SNV | | |
| | | RUNX1 | RUNX1:NM_001001890.3:exon1:c.C172A:p.H58N, | nonsynonymous | 200 | 0.443038 |
| | | | RUNX1:NM_001122607.2:exon1:c.C172A:p.H58N, | SNV | | |
| | | | RUNX1:NM_001754.5:exon4:c.C253A:p.H85N | | | |
| D084 | BM | NRAS | NRAS:NM_002524.5:exon3:c.A182G:p.Q61R | nonsynonymous | 61.02 | 0.3009709 |
| | | | | SNV | | |
| | | WRN | WRN:NM_000553.6:exon6:c.C650T:p.A217V | nonsynonymous | 105.08 | 0.4948454 |
| | | | | SNV | | |
| D085 | BM | DNMT3A | DNMT3A:NM_001320893.1:exon3:c.G483A:p.W161X, | stopgain | 200 | 0.3820513 |
| | | | DNMT3A:NM_001375819.1:exon3:c.G270A:p.W90X, | | | |
| | | | DNMT3A:NM_153759.3:exon4:c.G372A:p.W124X, | | | |
| | | | DNMT3A:NM_022552.5:exon8:c.G939A:p.W313X, | | | |
| | | | DNMT3A:NM_175629.2:exon8:c.G939A:p.W313X | | | |
| | | FLT3 | FLT3:NM_004119.3:exon20:c.G2503T:p.D835Y | nonsynonymous | 199.23 | 0.2053422 |
| | | | | SNV | | |
| | | KRAS | KRAS:NM_001369786.1:exon2:c.G35A:p.G12D, | nonsynonymous | 37.27 | 0.0823171 |
| | | | KRAS:NM_001369787.1:exon2:c.G35A:p.G12D, | SNV | | |
| | | | KRAS:NM_004985.5:exon2:c.G35A:p.G12D, | | | |
| | | | KRAS:NM_033360.4:exon2:c.G35A:p.G12D | | | |
| | | NPM1 | NPM1:NM_001355010.1:exon7:c.479_480insCTGC:p.W161Cfs*12, | frameshift | 120.21 | 0.3680982 |
| | | | NPM1:NM_001355007.1:exon10:c.668_669insCTGC:p.W224Cfs*12, | insertion | | |
| | | | NPM1:NM_199185.3:exon10:c.773_774insCTGC:p.W259Cfs*12, | | | |
| | | | NPM1:NM_002520.7:exon11:c.860_861insCTGC:p.W288Cfs*12, | | | |
| | | | NPM1:NM_001355006.1:exon12:c.860_861insCTGC:p.W288Cfs*12 | | | |
| | | RAD21 | RAD21:NM_006265.3:exon5:c.C394T:p.Q132X | stopgain | 108.59 | 0.3673469 |
| D086 | BM | DNMT3A | DNMT3A:NM_001320893.1:exon18:c.G2189A:p.R730H, | nonsynonymous | 200 | 0.4666667 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | EGFR | EGFR:NM_001346941.2:exon19:c.G2239A:p.D747N, | nonsynonymous | 150.09 | 0.4382716 |
| | | | EGFR:NM_001346897.2:exon24:c.G2905A:p.D969N, | SNV | | |
| | | | EGFR:NM_001346899.1:exon24:c.G2905A:p.D969N, | | | |
| | | | EGFR:NM_001346898.2:exon25:c.G3040A:p.D1014N, | | | |
| | | | EGFR:NM_001346900.2:exon25:c.G2881A:p.D961N, | | | |
| | | | EGFR:NM_005228.5:exon25:c.G3040A:p.D1014N | | | |
| | | NPM1 | NPM1:NM_001355010.1:exon7:c.478_479insTCTG:p.W161Cfs*12, | frameshift | 37.88 | 0.3103448 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | TET2 | TET2:NM_001127208.3:exon3:c.2117delA:p.Q706Rfs*45, | frameshift | 200 | 0.7886179 |
| | | | TET2:NM_017628.4:exon3:c.2117delA:p.Q706Rfs*45 | deletion | | |
| D087 | BM | CEBPA | CEBPA:NM_001285829.1:exon1:c.594_595insCTG:p.L198_{—} | nonframeshift | 127.49 | 0.4580153 |
| | | | T199insL, CEBPA:NM_001287424.2:exon1:c.1056_{—} | insertion | | |
| | | | 1057insCTG:p.L352_T353insL, CEBPA:NM_{—} | | | |
| | | | 001287435.1:exon1:c.909_910insCTG:p.L303_T304insL, | | | |
| | | | CEBPA:NM_004364.5:exon1:c.951_952insCTG:p.L317_T318insL | | | |
| | | CEBPA | CEBPA:NM_001287424.2:exon1:c.361dupC:p.R121Pfs*22, | frameshift | 175.58 | 0.4880952 |
| | | | CEBPA:NM_001287435.1:exon1:c.214dupC:p.R72Pfs*22, | insertion | | |
| | | | CEBPA:NM_004364.5:exon1:c.256dupC:p.R86Pfs*22 | | | |
| | | NRAS | NRAS:NM_002524.5:exon3:c.A183T:p.Q61H | nonsynonymous | 19.46 | 0.08125 |
| | | | | SNV | | |
| | | NRAS | NRAS:NM_002524.5:exon2:c.G38A:p.G13D | nonsynonymous | 133.24 | 0.28125 |
| | | | | SNV | | |
| D088 | PB | CEBPA | CEBPA:NM_001287424.2:exon1:c.297dupC:p.S100Qfs*43, | frameshift | 200 | 0.5133136 |
| | | | CEBPA:NM_001287435.1:exon1:c.150dupC:p.S51Qfs*43, | insertion | | |
| | | | CEBPA:NM_004364.5:exon1:c.192dupC:p.S65Qfs*43 | | | |
| | | CEBPA | CEBPA:NM_001285829.1:exon1:c.T647C:p.L216P, | nonsynonymous | 200 | 0.476087 |
| | | | CEBPA:NM_001287424.2:exon1:c.T1109C:p.L370P, | SNV | | |
| | | | CEBPA:NM_001287435.1:exon1:c.T962C:p.L321P, | | | |
| | | | CEBPA:NM_004364.5:exon1:c.T1004C:p.L335P | | | |
| | | IDH2 | IDH2:NM_001290114.2:exon2:c.G29A:p.R10Q, | nonsynonymous | 200 | 0.4636678 |
| | | | IDH2:NM_001289910.1:exon4:c.G263A:p.R88Q, | SNV | | |
| | | | IDH2:NM_002168.4:exon4:c.G419A:p.R140Q | | | |
| | | KIT | KIT:NM_000222.3:exon17:c.A2447T:p.D816V, | nonsynonymous | 108.52 | 0.3313953 |
| | | | KIT:NM_001093772.2:exon17:c.A2435T:p.D812V, | SNV | | |
| | | | KIT:NM_001385284.1:exon17:c.A2450T:p.D817V, | | | |
| | | | KIT:NM_001385285.1:exon17:c.A2444T:p.D815V, | | | |
| | | | KIT:NM_001385286.1:exon17:c.A2432T:p.D811V, | | | |
| | | | KIT:NM_001385288.1:exon17:c.A2438T:p.D813V, | | | |
| | | | KIT:NM_001385290.1:exon17:c.A2447T:p.D816V, | | | |
| | | | KIT:NM_001385292.1:exon17:c.A2435T:p.D812V | | | |
| | | WT1 | WT1:NM_001367854.1:exon5:c.G185A:p.C62Y, | nonsynonymous | 103.96 | 0.2040134 |
| | | | WT1:NM_000378.6:exon8:c.G1322A:p.C441Y, | SNV | | |
| | | | WT1:NM_001198552.2:exon8:c.G671A:p.C224Y, | | | |
| | | | WT1:NM_001198551.1:exon9:c.G722A:p.C241Y, | | | |
| | | | WT1:NM_024424.5:exon9:c.G1373A:p.C458Y, | | | |
| | | | WT1:NM_024426.6:exon9:c.G1373A:p.C458Y | | | |
| D089 | BM | KIT | KIT:NM_000222.3:exon17:c.A2447T:p.D816V, | nonsynonymous | 200 | 0.4078624 |
| | | | KIT:NM_001093772.2:exon17:c.A2435T:p.D812V, | SNV | | |
| | | | KIT:NM_001385284.1:exon17:c.A2450T:p.D817V, | | | |
| | | | KIT:NM_001385285.1:exon17:c.A2444T:p.D815V, | | | |
| | | | KIT:NM_001385286.1:exon17:c.A2432T:p.D811V, | | | |
| | | | KIT:NM_001385288.1:exon17:c.A2438T:p.D813V, | | | |
| | | | KIT:NM_001385290.1:exon17:c.A2447T:p.D816V, | | | |
| | | | KIT:NM_001385292.1:exon17:c.A2435T:p.D812V | | | |
| | | SMC3 | SMC3:NM_005445.4:exon25:c.C3007T:p.R1003C | nonsynonymous | 94.13 | 0.1309771 |
| | | | | SNV | | |
| | | SRSF2 | SRSF2:NM_001195427.2:exon1:c.C284T:p.P95L, | nonsynonymous | 18.47 | 0.0534351 |
| | | | SRSF2:NM_003016.4:exon1:c.C284T:p.P95L | SNV | | |
| D090 | BM | IDH1 | IDH1:NM_001282386.1:exon4:c.G395A:p.R132H, | nonsynonymous | 93.05 | 0.3137255 |
| | | | IDH1:NM_001282387.1:exon4:c.G395A:p.R132H, | SNV | | |
| | | | IDH1:NM_005896.4:exon4:c.G395A:p.R132H | | | |
| | | STAG2 | STAG2:NM_001375375.1:exon30:c.T3395G:p.L1132X, | stopgain | 200 | 0.4661017 |
| | | | STAG2:NM_006603.5:exon30:c.T3395G:p.L1132X, | | | |
| | | | STAG2:NM_001042749.2:exon31:c.T3395G:p.L1132X, | | | |
| | | | STAG2:NM_001042750.2:exon31:c.T3395G:p.L1132X, | | | |
| | | | STAG2:NM_001042751.2:exon31:c.T3395G:p.L1132X, | | | |
| | | | STAG2:NM_001282418.2:exon31:c.T3395G:p.L1132X | | | |
| D091 | BM | DNMT3A | DNMT3A:NM_001320893.1:exon18:c.G2189A:p.R730H, | nonsynonymous | 184.51 | 0.453125 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | NPM1 | NPM1:NM_001355010.1:exon7:c.478_479insTCTG:p.W161Cfs*12, | frameshift | 35.79 | 0.483871 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | PRPF8 | PRPF8:NM_006445.4:exon37:c.T5963C:p.L1988S | nonsynonymous | 102.07 | 0.4946237 |
| | | | | SNV | | |
| | | PTPN11 | PTPN11:NM_001330437.2:exon3:c.G205A:p.E69K, | nonsynonymous | 23 | 0.0842697 |
| | | | PTPN11:NM_001374625.1:exon3:c.G202A:p.E68K, | SNV | | |
| | | | PTPN11:NM_002834.5:exon3:c.G205A:p.E69K, | | | |
| | | | PTPN11:NM_080601.3:exon3:c.G205A:p.E69K | | | |
| D092 | BM | CEBPA | CEBPA:NM_001285829.1:exon1:c.571_572insAGA:p.E190_{—} | nonframeshift | 186.9 | 0.584507 |
| | | | T191insK, CEBPA:NM_001287424.2:exon1:c.1033_{—} | insertion | | |
| | | | 1034insAGA:p.E344_T345insK, CEBPA:NM_{—} | | | |
| | | | 001287435.1:exon1:c.886_887insAGA:p.E295_T296insK, | | | |
| | | | CEBPA:NM_004364.5:exon1:c.928_929insAGA:p.E309_T310insK | | | |
| D093 | BM | NRAS | NRAS:NM_002524.5:exon3:c.C181A:p.Q61K | nonsynonymous | 56.26 | 0.2906977 |
| | | | | SNV | | |
| | | WT1 | WT1:NM_000378.6:exon6:c.1099_1100insACTCTTG:p.V367Dfs*8, | frameshift | 15.95 | 0.1046512 |
| | | | WT1:NM_001198552.2:exon6:c.448_449insACTCTTG:p.V150Dfs*8, | insertion | | |
| | | | WT1:NM_001198551.1:exon7:c.499_500insACTCTTG:p.V167Dfs*8, | | | |
| | | | WT1:NM_024424.5:exon7:c.1150_1151insACTCTTG:p.V384Dfs*8, | | | |
| | | | WT1:NM_024426.6:exon7:c.1150_115linsACTCTTG:p.V384Dfs*8 | | | |
| D094 | BM | DNMT3A | DNMT3A:NM_001320893.1:exon18:c.C2188T:p.R730C, | nonsynonymous | 200 | 0.4708171 |
| | | | DNMT3A:NM_001375819.1:exon18:c.C1975T:p.R659C, | SNV | | |
| | | | DNMT3A:NM_153759.3:exon19:c.C2077T:p.R693C, | | | |
| | | | DNMT3A:NM_022552.5:exon23:c.C2644T:p.R882C, | | | |
| | | | DNMT3A:NM_175629.2:exon23:c.C2644T:p.R882C | | | |
| | | FLT3 | FLT3:NM_004119.3:exon20:c.G2503T:p.D835Y | nonsynonymous | 41.19 | 0.1818182 |
| | | | | SNV | | |
| | | FLT3 | FLT3:NM_004119.3:exon14:c.1778_{—} | nonframeshift | 44.01 | 0.1206897 |
| | | | 1779insGGATAATGAGTACTTCTACGTTGA:p.V592_{—} | insertion | | |
| | | | D593insEDNEYFYV | | | |
| | | NPM1 | NPM1:NM_001355010.1:exon7:c.478_479insTCTG:p.W161Cfs*12, | frameshift | 21.11 | 0.2325581 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | RB1 | RB1:NM_000321.3:exon7:c.T643C:p.S215P | nonsynonymous | 84.75 | 0.4871795 |
| | | | | SNV | | |
| D095 | BM | IDH2 | IDH2:NM_001290114.2:exon2:c.G125A:p.R42K, | nonsynonymous | 76.16 | 0.3557692 |
| | | | IDH2:NM_001289910.1:exon4:c.G359A:p.R120K, | SNV | | |
| | | | IDH2:NM_002168.4:exon4:c.G515A:p.R172K | | | |
| | | JAK1 | JAK1:NM_001321852.2:exon7:c.A938G:p.Y313C, | nonsynonymous | 105.41 | 0.5164835 |
| | | | JAK1:NM_001321856.1:exon7:c.A938G:p.Y313C, | SNV | | |
| | | | JAK1:NM_001321857.2:exon7:c.A938G:p.Y313C, | | | |
| | | | JAK1:NM_002227.4:exon7:c.A938G:p.Y313C, | | | |
| | | | JAK1:NM_001320923.1:exon8:c.A938G:p.Y313C, | | | |
| | | | JAK1:NM_001321854.2:exon8:c.A938G:p.Y313C, | | | |
| | | | JAK1:NM_001321855.2:exon8:c.A938G:p.Y313C, | | | |
| | | | JAK1:NM_001321853.2:exon9:c.A938G:p.Y313C | | | |
| D096 | BM | CEBPA | CEBPA:NM_001285829.1:exon1:c.570_571insGAG:p.E190_{—} | nonframeshift | 74.61 | 0.4473684 |
| | | | T191insE, CEBPA:NM_001287424.2:exon1:c.1032_{—} | insertion | | |
| | | | 1033insGAG:p.E344_T345insE, CEBPA:NM_{—} | | | |
| | | | 001287435.1:exon1:c.885_886insGAG:p.E295_T296insE, | | | |
| | | | CEBPA:NM_004364.5:exon1:c.927_928insGAG:p.E309_T310insE | | | |
| | | CEBPA | CEBPA:NM_001287424.2:exon1:c.208delC:p.R70Gfs*125, | frameshift | 104.73 | 0.4032258 |
| | | | CEBPA:NM_001287435.1:exon1:c.61delC:p.R21Gfs*125, | deletion | | |
| | | | CEBPA:NM_004364.5:exon1:c.103delC:p.R35Gfs*125 | | | |
| | | WT1 | WT1:NM_001367854.1:exon5:c.C211T:p.R71W, | nonsynonymous | 198.32 | 0.447619 |
| | | | WT1:NM_000378.6:exon8:c.C1348T:p.R450W, | SNV | | |
| | | | WT1:NM_001198552.2:exon8:c.C697T:p.R233W, | | | |
| | | | WT1:NM_001198551.1:exon9:c.C748T:p.R250W, | | | |
| | | | WT1:NM_024424.5:exon9:c.C1399T:p.R467W, | | | |
| | | | WT1:NM_024426.6:exon9:c.C1399T:p.R467W | | | |
| D097 | BM | WT1 | WT1:NM_000378.6:exon6:c.1102delinsGG:p.R368Gfs*5, | frameshift | 200 | 0.4040816 |
| | | | WT1:NM_001198552.2:exon6:c.451delinsGG:p.R151Gfs*5, | substitution | | |
| | | | WT1:NM_001198551.1:exon7:c.502delinsGG:p.R168Gfs*5, | | | |
| | | | WT1:NM_024424.5:exon7:c.1153delinsGG:p.R385Gfs*5, | | | |
| | | | WT1:NM_024426.6:exon7:c.1153delinsGG:p.R385Gfs*5 | | | |
| D098 | BM | CREBBP | CREBBP:NM_001079846.1:exon12:c.A2305C:p.S769R, | nonsynonymous | 200 | 0.4367816 |
| | | | CREBBP:NM_004380.3:exon13:cA2419C:p.S807R | SNV | | |
| | | KIT | KIT:NM_000222.3:exon17:c.G2446C:p.D816H, | nonsynonymous | 113.96 | 0.3511905 |
| | | | KIT:NM_001093772.2:exon17:c.G2434C:p.D812H, | SNV | | |
| | | | KIT:NM_001385284.1:exon17:c.G2449C:p.D817H, | | | |
| | | | KIT:NM_001385285.1:exon17:c.G2443C:p.D815H, | | | |
| | | | KIT:NM_001385286.1:exon17:c.G2431C:p.D811H, | | | |
| | | | KIT:NM_001385288.1:exon17:c.G2437C:p.D813H, | | | |
| | | | KIT:NM_001385290.1:exon17:c.G2446C:p.D816H, | | | |
| | | | KIT:NM_001385292.1:exon17:c.G2434C:p.D812H | | | |
| D099 | BM | BCORL1 | BCORL1:NM_021946.5:exon12:c.C4827G:p.Y1609X, | stopgain | 100.96 | 0.8863636 |
| | | | BCORL1:NM_001379450.1:exon13:c.C5049G:p.Y1683X, | | | |
| | | | BCORL1:NM_001379451.1:exon13:c.C5049G:p.Y1683X, | | | |
| | | | BCORL1:NM_001184772.3:exon14:c.C5049G:p.Y1683X | | | |
| | | DNMT3A | DNMT3A:NM_001320893.1:exon18:c.C2188T:p.R730C, | nonsynonymous | 200 | 0.4605809 |
| | | | DNMT3A:NM_001375819.1:exon18:c.C1975T:p.R659C, | SNV | | |
| | | | DNMT3A:NM_153759.3:exon19:c.C2077T:p.R693C, | | | |
| | | | DNMT3A:NM_022552.5:exon23:c.C2644T:p.R882C, | | | |
| | | | DNMT3A:NM_175629.2:exon23:c.C2644T:p.R882C | | | |
| | | IDH2 | IDH2:NM_001290114.2:exon2:c.G29A:p.R10Q, | nonsynonymous | 122.02 | 0.4393939 |
| | | | IDH2:NM_001289910.1:exon4:c.G263A:p.R88Q, | SNV | | |
| | | | IDH2:NM_002168.4:exon4:c.G419A:p.R140Q | | | |
| | | RAD21 | RAD21:NM_006265.3:exon10:c.A1216T:p.K406X | stopgain | 75.46 | 0.3584906 |
| | | RUNX1 | RUNX1:NM_001001890.3:exon6:c.955dupC:p.R319Pfs*254, | frameshift | 74.14 | 0.4117647 |
| | | | RUNX1:NM_001754.5:exon9:c.1036dupC:p.R346Pfs*254 | insertion | | |
| D100 | BM | HNRNPK | HNRNPK:NM_001318186.1:exon4:c.68delC:p.P23Lfs*35, | frameshift | 100.14 | 0.3571429 |
| | | | HNRNPK:NM_001318187.1:exon4:c.68delC:p.P23Lfs*35, | deletion | | |
| | | | HNRNPK:NM_001318188.1:exon4:c.68delC:p.P23Lfs*35, | | | |
| | | | HNRNPK:NM_002140.4:exon4:c.68delC:p.P23Lfs*35, | | | |
| | | | HNRNPK:NM_031262.3:exon4:c.68delC:p.P23Lfs*35, | | | |
| | | | HNRNPK:NM_031263.4:exon4:c.68delC:p.P23Lfs*35 | | | |
| D101 | BM | IDH2 | IDH2:NM_001290114.2:exon2:c.G29A:p.R10Q, | nonsynonymous | 71.27 | 0.4177215 |
| | | | IDH2:NM_001289910.1:exon4:c.G263A:p.R88Q, | SNV | | |
| | | | IDH2:NM_002168.4:exon4:c.G419A:p.R140Q | | | |
| | | NPM1 | NPM1:NM_001355010.1:exon7:c.478_479insTCTG:p.W161Cfs*12, | frameshift | 29.98 | 0.3939394 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D102 | BM | ETNK1 | ETNK1:NM_018638.5:exon3:c.A451T:p.I151F | nonsynonymous | 28.75 | 0.2063492 |
| | | | | SNV | | |
| | | IDH2 | IDH2:NM_001290114.2:exon2:c.G125A:p.R42K, | nonsynonymous | 80.09 | 0.2727273 |
| | | | IDH2:NM_001289910.1:exon4:c.G359A:p.R120K, | SNV | | |
| | | | IDH2:NM_002168.4:exon4:c.G515A:p.R172K | | | |
| D103 | BM | DNMT3A | DNMT3A:NM_001320893.1:exon18:c.G2189A:p.R730H, | nonsynonymous | 200 | 0.4276094 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | KRAS | KRAS:NM_001369786.1:exon2:c.G35C:p.G12A, | nonsynonymous | 61.67 | 0.3714286 |
| | | | KRAS:NM_001369787.1:exon2:c.G35C:p.G12A, | SNV | | |
| | | | KRAS:NM_004985.5:exon2:c.G35C:p.G12A, | | | |
| | | | KRAS:NM_033360.4:exon2:c.G35C:p.G12A | | | |
| | | NRAS | NRAS:NM_002524.5:exon2:c.G35A:p.G12D | nonsynonymous | 25.97 | 0.1066667 |
| | | | | SNV | | |
| | | RUNX1 | RUNX1:NM_001001890.3:exon2:c.398_412del:p.D133_V137del, | nonframeshift | 75.24 | 0.3394495 |
| | | | RUNX1:NM_001122607.2:exon2:c.398_412del:p.D133_V137del, | deletion | | |
| | | | RUNX1:NM_001754.5:exon5:c.479_493del:p.D160_V164del | | | |
| D104 | BM | BRINP3 | BRINP3:NM_001317188.1:exon7:c.C1655T:p.P552L, | nonsynonymous | 200 | 0.5560748 |
| | | | BRINP3:NM_199051.3:exon8:c.C1961T:p.P654L | SNV | | |
| | | IDH1 | IDH1:NM_001282386.1:exon4:c.C394T:p.R132C, | nonsynonymous | 77.65 | 0.3653846 |
| | | | IDH1:NM_001282387.1:exon4:c.C394T:p.R132C, | SNV | | |
| | | | IDH1:NM_005896.4:exon4:c.C394T:p.R132C | | | |
| | | NPM1 | NPM1:NM_001355010.1:exon7:c.481_482insGCCA:p.W161Cfs*12, | frameshift | 76.43 | 0.5689655 |
| | | | NPM1:NM_001355007.1:exon10:c.670_671insGCCA:p.W224Cfs*12, | insertion | | |
| | | | NPM1:NM_199185.3:exon10:c.775_776insGCCA:p.W259Cfs*12, | | | |
| | | | NPM1:NM_002520.7:exon11:c.862_863insGCCA:p.W288Cfs*12, | | | |
| | | | NPM1:NM_001355006.1:exon12:c.862_863insGCCA:p.W288Cfs*12 | | | |
| D105 | BM | CEBPA | CEBPA:NM_001287424.2:exon1:c.423dupT:p.D142*, | stopgain | 16.89 | 0.2285714 |
| | | | CEBPA:NM_001287435.1:exon1:c.276dupT:p.D93*, | | | |
| | | | CEBPA:NM_004364.5:exon1:c.318dupT:p.D107* | | | |
| | | NRAS | NRAS:NM_002524.5:exon3:c.A183C:p.Q61H | nonsynonymous | 102.15 | 0.3661972 |
| | | | | SNV | | |
| D106 | BM | DNMT3A | DNMT3A:NM_001320893.1:exon18:c.G2189A:p.R730H, | nonsynonymous | 200 | 0.4419552 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | NPM1 | NPM1:NM_001355010.1:exon7:c.478_479insTCTG:p.W161Cfs*12, | frameshift | 64.52 | 0.373494 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | PTPN11 | PTPN11:NM_001330437.2:exon3:c.G181C:p.D61H, | nonsynonymous | 28.87 | 0.0578313 |
| | | | PTPN11:NM_001374625.1:exon3:c.G178C:p.D60H, | SNV | | |
| | | | PTPN11:NM_002834.5:exon3:c.G181C:p.D61H, | | | |
| | | | PTPN11:NM_080601.3:exon3:c.G181C:p.D61H | | | |
| | | RUNX1 | RUNX1:NM_001001890.3:exon4:c.C656T:p.T219M, | nonsynonymous | 150.55 | 0.4640523 |
| | | | RUNX1:NM_001122607.2:exon4:c.C656T:p.T219M, | SNV | | |
| | | | RUNX1:NM_001754.5:exon7:c.C737T:p.T246M | | | |
| D107 | BM | NOTCH1 | NOTCH1:NM_017617.5:exon25:c.T4420G:p.W1474G | nonsynonymous | 29.62 | 0.0621762 |
| | | | | SNV | | |
| | | TP53 | TP53:NM_001126115.1:exon4:c.C447A:p.D149E, | nonsynonymous | 200 | 0.9124236 |
| | | | TP53:NM_001126116.1:exon4:c.C447A:p.D149E, | SNV | | |
| | | | TP53:NM_001126117.1:exon4:c.C447A:p.D149E, | | | |
| | | | TP53:NM_001276697.2:exon4:c.C366A:p.D122E, | | | |
| | | | TP53:NM_001276698.2:exon4:c.C366A:p.D122E, | | | |
| | | | TP53:NM_0012766992:exon4:c.C366A:p.D122E, | | | |
| | | | TP53:NM_001126118.1:exon7:c.C726A:p.D242E, | | | |
| | | | TP53:NM_000546.6:exon8:c.C843A:p.D281E, | | | |
| | | | TP53:NM_001126112.2:exon8:c.C843A:p.D281E, | | | |
| | | | TP53:NM_001126113.2:exon8:c.C843A:p.D281E, | | | |
| | | | TP53:NM_001126114.2:exon8:c.C843A:p.D281E, | | | |
| | | | TP53:NM_001276695.2:exon8:c.C726A:p.D242E, | | | |
| | | | TP53:NM_001276696.2:exon8:c.C726A:p.D242E, | | | |
| | | | TP53:NM_001276760.2:exon8:c.C726A:p.D242E, | | | |
| | | | TP53:NM_001276761.2:exon8:c.C726A:p.D242E | | | |
| D108 | BM | ATRX | ATRX:NM_138270.4:exon19:c.G5114T:p.R1705M, | nonsynonymous | 179.3 | 0.4467005 |
| | | | ATRX:NM_000489.6:exon20:c.G5228T:p.R1743M | SNV | | |
| | | CTCF | CTCF:NM_001363916.1:exon3:c.604dupA:p.T204Nfs*26, | frameshift | 112.7 | 0.2310469 |
| | | | CTCF:NM_006565.4:exon3:c.604dupA:p.T204Nfs*26 | insertion | | |
| | | FLT3 | FLT3:NM_004119.3:exon20:c.2508_2510del:p.I836del | nonframeshift | 67.01 | 0.1921182 |
| | | | | deletion | | |
| | | IDH2 | IDH2:NM_001290114.2:exon2:c.C28T:p.R10W, | nonsynonymous | 53.02 | 0.16 |
| | | | IDH2:NM_001289910.1:exon4:c.C262T:p.R88W, | SNV | | |
| | | | IDH2:NM_002168.4:exon4:c.C418T:pR140W | | | |
| | | NRAS | NRAS:NM_002524.5:exon3:c.A182G:p.Q61R | nonsynonymous | 68.16 | 0.2450331 |
| | | | | SNV | | |
| | | RAD21 | RAD21:NM_006265.3:exon12:c.1537_1538insATCT:p.C513Yfs*25 | frameshift | 33.45 | 0.12 |
| | | | | insertion | | |
| | | U2AF1; | U2AF1:NM_001025203.1:exon2:c.G104A:p.R35Q, | nonsynonymous | 70.26 | 0.25 |
| | | U2AF1L5 | U2AF1L5:NM_001320646.2:exon2:c.G104A:p.R35Q, | SNV | | |
| | | | U2AF1L5:NM_001320648.2:exon2:c.G104A:p.R35Q, | | | |
| | | | U2AF1L5:NM_001320650.2:exon2:c.G19A:p.G7S, | | | |
| | | | U2AF1:NM_006758.3:exon2:c.G104A:p.R35Q | | | |
| | | WT1 | WT1:NM_000378.6:exon6:c.1108_1109insTCGG:p.A370Vfs*4, | frameshift | 43.58 | 0.123348 |
| | | | WT1:NM_001198552.2:exon6:c.457_458insTCGG:p.A153Vfs*4, | insertion | | |
| | | | WT1:NM_001198551.1:exon7:c.508_509insTCGG:p.A170Vfs*4, | | | |
| | | | WT1:NM_024424.5:exon7:c.1159_1160insTCGG:p.A387Vfs*4, | | | |
| | | | WT1:NM_024426.6:exon7:c.1159_1160insTCGG:p.A387Vfs*4 | | | |
| D109 | BM | DNMT3A | DNMT3A:NM_001320893.1:exon18:c.G2189A:p.R730H, | nonsynonymous | 200 | 0.5122898 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | NPM1 | NPM1:NM_001355010.1:exon7:c.478_479insTCTG:p.W161Cfs*12, | frameshift | 68.29 | 0.3333333 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D110 | BM | ATM | ATM:NM_000051.4:exon18:c.C2770T:p.R924W, | nonsynonymous | 200 | 0.5571956 |
| | | | ATM:NM_001351834.2:exon19:c.C2770T:p.R924W | SNV | | |
| | | DNMT3A | DNMT3A:NM_001320893.1:exon18:c.G2189A:p.R730H, | nonsynonymous | 200 | 0.4364723 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | NPM1 | NPM1:NM_001355010.1:exon7:c.478_479insTCTG:p.W161Cfs*12, | frameshift | 55.84 | 0.3076923 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D111 | BM | BCORL1 | BCORL1:NM_001379450.1:exon4:c.C1468T:p.L490F, | nonsynonymous | 200 | 0.4982206 |
| | | | BCORL1:NM_001379451.1:exon4:c.C1468T:p.L490F, | SNV | | |
| | | | BCORL1:NM_021946.5:exon4:c.C1468T:p.L490F, | | | |
| | | | BCORL1:NM_001184772.3:exon5:c.C1468T:p.L490F | | | |
| | | DNMT3A | DNMT3A:NM_001320893.1:exon18:c.G2189A:p.R730H, | nonsynonymous | 200 | 0.4310051 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | NPM1 | NPM1:NM_001355010.1:exon7:c.478_479insTCTG:p.W161Cfs*12, | frameshift | 100.86 | 0.4247788 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D112 | BM | BCORL1 | BCORL1:NM_001379450.1:exon4:c.2524_2528del:p.L842Hfs*43, | frameshift | 193.24 | 0.2971429 |
| | | | BCORL1:NM_001379451.1:exon4:c.2524_2528del:p.L842Hfs*43, | deletion | | |
| | | | BCORL1:NM_021946.5:exon4:c.2524_2528del:p.L842Hfs*43, | | | |
| | | | BCORL1:NM_001184772.3:exon5:c.2524_2528del:p.L842Hfs*43 | | | |
| | | BRCA1 | NM_007300.4:exon13:c.4423 + 1G > T | splice site | 200 | 0.4972826 |
| | | | | mutation | | |
| | | DNMT3A | DNMT3A:NM_001320893.1:exon18:c.G2189A:p.R730H, | nonsynonymous | 200 | 0.3338843 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | RUNX1 | RUNX1:NM_001001890.3:exon6:c.1129dupC:p.H377Pfs*196, | frameshift | 73.35 | 0.2453988 |
| | | | RUNX1:NM_001754.5:exon9:c.1210dupC:p.H404Pfs*196 | insertion | | |
| D113 | BM | DNMT3A | DNMT3A:NM_001320893.1:exon18:c.G2189A:p.R730H, | nonsynonymous | 200 | 0.5243243 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | KIT | KIT:NM_000222.3:exon17:c.A2447T:p.D816V, | nonsynonymous | 200 | 0.754386 |
| | | | KIT:NM_001093772.2:exon17:c.A2435T:p.D812V, | SNV | | |
| | | | KIT:NM_001385284.1:exon17:c.A2450T:p.D817V, | | | |
| | | | KIT:NM_001385285.1:exon17:c.A2444T:p.D815V, | | | |
| | | | KIT:NM_001385286.1:exon17:c.A2432T:p.D811V, | | | |
| | | | KIT:NM_001385288.1:exon17:c.A2438T:p.D813V, | | | |
| | | | KIT:NM_001385290.1:exon17:c.A2447T:p.D816V, | | | |
| | | | KIT:NM_001385292.1:exon17:c.A2435T:p.D812V | | | |
| | | NPM1 | NPM1:NM_001355010.1:exon7:c.478_479insTCTG:p.W161Cfs*12, | frameshift | 39.53 | 0.5714286 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D114 | BM | BCORL1 | BCORL1:NM_001379450.1:exon9:c.C4336T:p.R1446X, | stopgain | 200 | 0.3037383 |
| | | | BCORL1:NM_001379451.1:exon9:c.C4336T:p.R1446X, | | | |
| | | | BCORL1:NM_001184772.3:exon10:c.C4336T:p.R1446X | | | |
| | | WRN | WRN:NM_000553.6:exon7:c.G655T:p.A219S | nonsynonymous | 200 | 0.464876 |
| | | | | SNV | | |
| D115 | BM | DNMT3A | DNMT3A:NM_001320893.1:exon18:c.G2189A:p.R730H, | nonsynonymous | 200 | 0.3886097 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | MSH2 | MSH2:NM_000251.3:exon2:c.G232A:p.V78I, | nonsynonymous | 200 | 0.5426357 |
| | | | MSH2:NM_001258281.1:exon3:c.G34A:p.V12I | SNV | | |
| | | NPM1 | NPM1:NM_001355010.1:exon7:c.478_479insTCTG:p.W161Cfs*12, | frameshift | 69.09 | 0.3882353 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | PTPN11 | PTPN11:NM_001330437.2:exon13:c.G1520C:p.G507A, | nonsynonymous | 200 | 0.3746835 |
| | | | PTPN11:NM_001374625.1:exon13:c.G1505C:p.G502A, | SNV | | |
| | | | PTPN11:NM_002834.5:exon13:c.G1508C:p.G503A | | | |
| | | SMC1A | SMC1A:NM_006306.4:exon11:c.C1756T:p.R586W, | nonsynonymous | 200 | 0.3987138 |
| | | | SMC1A:NM_001281463.1:exon12:c.C1690T:p.R564W | SNV | | |
| D116 | BM | SH2B3 | SH2B3:NM_001291424.1:exon6:c.C667T:p.R223C, | nonsynonymous | 200 | 0.5290859 |
| | | | SH2B3:NM_005475.3:exon7:c.C1273T:p.R425C | SNV | | |
| D118 | BM | DNMT3A | DNMT3A:NM_001320893.1:exon18:c.G2189A:p.R730H, | nonsynonymous | 200 | 0.4785714 |
| | | | DNMT3A:NM_001375819.1:exon18:c.G1976A:p.R659H, | SNV | | |
| | | | DNMT3A:NM_153759.3:exon19:c.G2078A:p.R693H, | | | |
| | | | DNMT3A:NM_022552.5:exon23:c.G2645A:p.R882H, | | | |
| | | | DNMT3A:NM_175629.2:exon23:c.G2645A:p.R882H | | | |
| | | NPM1 | NPM1:NM_001355010.1:exon7:c.478_479insTCTG:p.W161Cfs*12, | frameshift | 55.76 | 0.5333333 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D119 | BM | KRAS | KRAS:NM_001369786.1:exon2:c.35_36delinsCA:p.G12A, | nonframeshift | 78.96 | 0.3253968 |
| | | | KRAS:NM_001369787.1:exon2:c.35_36delinsCA:p.G12A, | substitution | | |
| | | | KRAS:NM_004985.5:exon2:c.35_36delinsCA:p.G12A, | | | |
| | | | KRAS:NM_033360.4:exon2:c.35_36delinsCA:p.G12A | | | |
| | | RUNX1 | RUNX1:NM_001001890.3:exon2:c.301_302insGGCA:p.T101Rfs*11, | frameshift | 58.69 | 0.2912621 |
| | | | RUNX1:NM_001122607.2:exon2:c.301_302insGGCA:p.T101Rfs*11, | insertion | | |
| | | | RUNX1:NM_001754.5:exon5:c.382_383insGGCA:p.T128Rfs*11 | | | |
| | | STAG2 | STAG2:NM_001375375.1:exon19:c.1908delinsTAA:p.H637Nfs*15, | frameshift | 152.74 | 0.7654321 |
| | | | STAG2:NM_006603.5:exon19:c.1908delinsTAA:p.H637Nfs*15, | substitution | | |
| | | | STAG2:NM_001042749.2:exon20:c.1908delinsTAA:p.H637Nfs*15, | | | |
| | | | STAG2:NM_001042750.2:exon20:c.1908delinsTAA:p.H637Nfs*15, | | | |
| | | | STAG2:NM_001042751.2:exon20:c.1908delinsTAA:p.H637Nfs*15, | | | |
| | | | STAG2:NM_001282418.2:exon20:c.1908delinsTAA:p.H637Nfs*15 | | | |
| D121 | BM | NPM1 | NPM1:NM_001355010.1:exon7:c.478_479insTCTG:p.W161Cfs*12, | frameshift | 54.83 | 0.5 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D122 | BM | BRAF | BRAF:NM_001378470.1:exon7:c.C937T:p.R313X, | stopgain | 199.25 | 0.552795 |
| | | | BRAF:NM_001378475.1:exon7:c.C775T:p.R259X, | | | |
| | | | BRAF:NM_001354609.2:exon8:c.C1039T:p.R347X, | | | |
| | | | BRAF:NM_001374244.1:exon8:c.C1039T:p.R347X, | | | |
| | | | BRAF:NM_001374258.1:exon8:c.C1039T:p.R347X, | | | |
| | | | BRAF:NM_001378467.1:exon8:c.C1048T:p.R350X, | | | |
| | | | BRAF:NM_001378468.1:exon8:c.C1039T:p.R347X, | | | |
| | | | BRAF:NM_001378469.1:exon8:c.C1039T:p.R347X, | | | |
| | | | BRAF:NM_001378471.1:exon8:c.C1039T:p.R347X, | | | |
| | | | BRAF:NM_001378472.1:exon8:c.C883T:p.R295X, | | | |
| | | | BRAF:NM_001378473.1:exon8:c.C883T:p.R295X, | | | |
| | | | BRAF:NM_001378474.1:exon8:c.C1039T:p.R347X, | | | |
| | | | BRAF:NM_004333.6:exon8:c.C1039T:p.R347X | | | |
| | | DNMT3A | DNMT3A:NM_001320893.1:exon18:c.C2188T:p.R730C, | nonsynonymous | 183.53 | 0.3775934 |
| | | | DNMT3A:NM_001375819.1:exon18:c.C1975T:p.R659C, | SNV | | |
| | | | DNMT3A:NM_153759.3:exon19:c.C2077T:p.R693C, | | | |
| | | | DNMT3A:NM_022552.5:exon23:c.C2644T:p.R882C, | | | |
| | | | DNMT3A:NM_175629.2:exon23:c.C2644T:p.R882C | | | |
| | | MLH1 | MLH1:NM_001167619.2:exon8:c.T53C:p.L18S, | nonsynonymous | 200 | 0.510101 |
| | | | MLH1:NM_001258273.1:exon8:c.T53C:p.L18S, | SNV | | |
| | | | MLH1:NM_001354615.1:exon8:c.T53C:p.L18S, | | | |
| | | | MLH1:NM_001354616.1:exon8:c.T53C:p.L18S, | | | |
| | | | MLH1:NM_001354629.1:exon8:c.T677C:p.L226S, | | | |
| | | | MLH1:NM_000249.4:exon9:c.T776C:p.L259S, | | | |
| | | | MLH1:NM_001167617.2:exon9:c.T482C:p.L161S, | | | |
| | | | MLH1:NM_001167618.2:exon9:c.T53C:p.L18S, | | | |
| | | | MLH1:NM_001258271.1:exon9:c.T776C:p.L259S, | | | |
| | | | MLH1:NM_001354617.1:exon9:c.T53C:p.L18S, | | | |
| | | | MLH1:NM_001354618.1:exon9:c.T53C:p.L18S, | | | |
| | | | MLH1:NM_001354620.1:exon9:c.T482C:p.L161S, | | | |
| | | | MLH1:NM_001354628.1:exon9:c.T776C:p.L259S, | | | |
| | | | MLH1:NM_001354630.1:exon9:c.T776C:p.L259S, | | | |
| | | | MLH1:NM_001258274.2:exon10:c.T53C:p.L18S, | | | |
| | | | MLH1:NM_001354619.1:exon10:c.T53C:p.L18S | | | |
| | | NRAS | NRAS:NM_002524.5:exon3:c.C181A:p.Q61K | nonsynonymous | 40.33 | 0.2535211 |
| | | | | SNV | | |
| | | RUNX1 | RUNX1:NM_001001890.3:exon3:c.G511A:p.D171N, | nonsynonymous | 101.99 | 0.4563107 |
| | | | RUNX1:NM_001122607.2:exon3:c.G511A:p.D171N, | SNV | | |
| | | | RUNX1:NM_001754.5:exon6:c.G592A:p.D198N | | | |
| | | TET2 | TET2:NM_001127208.3:exon9:c.C4075A:p.R1359S | nonsynonymous | 109.44 | 0.46875 |
| | | | | SNV | | |
| D123 | BM | CEBPA | CEBPA:NM_001285829.1:exon1:c.582_583insAAG:p.K194_{—} | nonframeshift | 114.21 | 0.3653846 |
| | | | V195insK, CEBPA:NM_001287424.2:exon1:c.1044_{—} | insertion | | |
| | | | 1045insAAG:p.K348_V349insK, CEBPA:NM_{—} | | | |
| | | | 001287435.1:exon1:c.897_898insAAG:p.K299_V300insK, | | | |
| | | | CEBPA:NM_004364.5:exon1:c.939_940insAAG:p.K313_V314insK | | | |
| | | CEBPA | CEBPA:NM_001287424.2:exon1:c.352delC:p.Q118Sfs*77, | frameshift | 161.47 | 0.4934211 |
| | | | CEBPA:NM_001287435.1:exon1:c.205delC:p.Q69Sfs*77, | deletion | | |
| | | | CEBPA:NM_004364.5:exon1:c.247delC:p.Q83Sfs*77 | | | |
| | | CREBBP | CREBBP:NM_001079846.1:exon7:c.G1646A:p.G549D, | nonsynonymous | 200 | 0.5649351 |
| | | | CREBBP:NM_004380.3:exon8:c.G1760A:p.G587D | SNV | | |
| | | NRAS | NRAS:NM_002524.5:exon2:c.G38T:p.G13V | nonsynonymous | 31.48 | 0.0830325 |
| | | | | SNV | | |
| | | NRAS | NRAS:NM_002524.5:exon3:c.A182G:p.Q61R | nonsynonymous | 58.62 | 0.1878453 |
| | | | | SNV | | |
| D124 | BM | STAG2 | STAG2:NM_001375375.1:exon15:c.1511delA:p.E505Sfs*9, | frameshift | 200 | 0.8099174 |
| | | | STAG2:NM_006603.5:exon15:c.1511delA:p.E505Sfs*9, | deletion | | |
| | | | STAG2:NM_001042749.2:exon16:c.1511delA:p.E505Sfs*9, | | | |
| | | | STAG2:NM_001042750.2:exon16:c.1511delA:p.E505Sfs*9, | | | |
| | | | STAG2:NM_001042751.2:exon16:c.1511delA:p.E505Sfs*9, | | | |
| | | | STAG2:NM_001282418.2:exon16:c.1511delA:p.E505Sfs*9 | | | |
| D125 | BM | ASXL2 | ASXL2:NM_001369347.1:exon9:c.484delA:p.I162*, | stopgain | 96.97 | 0.4411765 |
| | | | ASXL2:NM_001369346.1:exon10:c.1090delA:p.I364*, | | | |
| | | | ASXL2:NM_018263.6:exon11:c.1264delA:p.I422* | | | |
| | | FLT3 | FLT3:NM_004119.3:exon11:c.1333_1334delinsTT:p.A445L | nonframeshift | 155.46 | 0.4098361 |
| | | | | substitution | | |
| | | IDH2 | IDH2:NM_001290114.2:exon2:c.G29A:p.R10Q, | nonsynonymous | 107.04 | 0.4711538 |
| | | | IDH2:NM_001289910.1:exon4:c.G263A:p.R88Q, | SNV | | |
| | | | IDH2:NM_002168.4:exon4:c.G419A:p.R140Q | | | |
| | | SRSF2 | SRSF2:NM_001195427.2:exon1:c.284_307de1:p.P95_R102del, | nonframeshift | 38.04 | 0.3157895 |
| | | | SRSF2:NM_003016.4:exon1:c.284_307del:p.P95_R102del | deletion | | |
| D126 | BM | DNMT3A | DNMT3A:NM_001320893.1:exon18:c.C2188T:p.R730C, | nonsynonymous | 200 | 0.424183 |
| | | | DNMT3A:NM_001375819.1:exon18:c.C1975T:p.R659C, | SNV | | |
| | | | DNMT3A:NM_153759.3:exon19:c.C2077T:p.R693C, | | | |
| | | | DNMT3A:NM_022552.5:exon23:c.C2644T:p.R882C, | | | |
| | | | DNMT3A:NM_175629.2:exon23:c.C2644T:p.R882C | | | |
| | | NPM1 | NPM1:NM_001355010.1:exon7:c.478_479insTCTG:p.W161Cfs*12, | frameshift | 161 | 0.3384615 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D127 | BM | ASXL2 | ASXL2:NM_001369347.1:exon10:c.1224dupA:p.A409Sfs*38, | frameshift | 200 | 0.369637 |
| | | | ASXL2:NM_001369346.1:exon11:c.1830dupA:p.A611Sfs*38, | insertion | | |
| | | | ASXL2:NM_018263.6:exon12:c.2004dupA:p.A669Sfs*38 | | | |
| | | DNMT3A | DNMT3A:NM_001320893.1:exon10:c.C1320G:p.Y440X, | stopgain | 200 | 0.4383838 |
| | | | DNMT3A:NM_001375819.1:exon10:c.C1107G:p.Y369X, | | | |
| | | | DNMT3A:NM_153759.3:exon11:c.C1209G:p.Y403X, | | | |
| | | | DNMT3A:NM_022552.5:exon15:c.C1776G:p.Y592X, | | | |
| | | | DNMT3A:NM_175629.2:exon15:c.C1776G:p.Y592X | | | |
| | | DNMT3A | DNMT3A:NM_001320893.1:exon10:c.1318delT:p.Y440Tfs*59, | frameshift | 200 | 0.3765957 |
| | | | DNMT3A:NM_001375819.1:exon10:c.1105delT:p.Y369Tfs*59, | deletion | | |
| | | | DNMT3A:NM_153759.3:exon11:c.1207delT:p.Y403Tfs*59, | | | |
| | | | DNMT3A:NM_022552.5:exon15:c.1774delT:p.Y592Tfs*59, | | | |
| | | | DNMT3A:NM_175629.2:exon15:c.1774delT:p.Y592Tfs*59 | | | |
| | | NPM1 | NPM1:NM_001355010.1:exon7:c.478_479insTCTG:p.W161Cfs*12, | frameshift | 119.2 | 0.4027778 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | TET2 | TET2:NM_001127208.3:exon6:c.T3731C:p.L1244P | nonsynonymous | 200 | 0.2403698 |
| | | | | SNV | | |
| D128 | BM | CEBPA | CEBPA:NM_001287424.2:exon1:c.173dupC:p.H59Afs*84, | frameshift | 36.47 | 0.0954198 |
| | | | CEBPA:NM_001287435.1:exon1:c.26dupC:p.H10Afs*84, | insertion | | |
| | | | CEBPA:NM_004364.5:exon1:c.68dupC:p.H24Afs*84 | | | |
| | | NPM1 | NPM1:NM_001355010.1:exon7:c.478_479insTCTG:p.W161Cfs*12, | frameshift | 124.28 | 0.5 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | TET2 | TET2:NM_001127208.3:exon3:c.1334delT:p.L446*, | stopgain | 200 | 0.4375 |
| | | | TET2:NM_017628.4:exon3:c.1334delT:p.L446* | | | |
| | | TET2 | TET2:NM_001127208.3:exon6:c.T3697G:p.W1233G | nonsynonymous | 200 | 0.4811321 |
| | | | | SNV | | |
| D129 | BM | JAK2 | JAK2:NM_001322204.1:exon19:c.C2511G:p.N837K, | nonsynonymous | 200 | 0.5 |
| | | | JAK2:NM_001322195.1:exon21:c.C2958G:p.N986K, | SNV | | |
| | | | JAK2:NM_001322196.1:exon21:c.C2958G:p.N986K, | | | |
| | | | JAK2:NM_001322194.1:exon22:c.C2958G:p.N986K, | | | |
| | | | JAK2:NM_001322198.1:exon22:c.C1743G:p.N581K, | | | |
| | | | JAK2:NM_001322199.1:exon22:c.C1743G:p.N581K, | | | |
| | | | JAK2:NM_004972.4:exon22:c.C2958G:p.N986K | | | |
| | | NPM1 | NPM1:NM_001355010.1:exon7:c.478_479insTCTG:p.W161Cfs*12, | frameshift | 38.6 | 0.4146341 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | SRSF2 | SRSF2:NM_001195427.2:exon1:c.C284A:p.P95H, | nonsynonymous | 43.56 | 0.36 |
| | | | SRSF2:NM_003016.4:exon1:c.C284A:p.P95H | SNV | | |
| D130 | BM | ABL1 | ABL1:NM_005157.6:exon11:c.C2290T:p.R764W, | nonsynonymous | 59.44 | 0.490566 |
| | | | ABL1:NM_007313.2:exon11:c.C2347T:p.R783W | SNV | | |
| | | IDH1 | IDH1:NM_001282386.1:exon4:c.C394T:p.R132C, | nonsynonymous | 38.54 | 0.516129 |
| | | | IDH1:NM_001282387.1:exon4:c.C394T:p.R132C, | SNV | | |
| | | | IDH1:NM_005896.4:exon4:c.C394T:p.R132C | | | |
| D131 | BM | CSF1R | CSF1R:NM_001288705.3:exon10:c.T1564C:p.C522R, | nonsynonymous | 167.12 | 0.225058 |
| | | | CSF1R:NM_001375321.1:exon10:c.T1120C:p.C374R, | SNV | | |
| | | | CSF1R:NM_005211.3:exon11:c.T1564C:p.C522R, | | | |
| | | | CSF1R:NM_001349736.1:exon12:c.T1564C:p.C522R, | | | |
| | | | CSF1R:NM_001375320.1:exon12:c.T1564C:p.C522R | | | |
| | | EZH2 | EZH2:NM_001203249.2:exon15:c.G1708A:p.V570M, | nonsynonymous | 197.35 | 0.3690037 |
| | | | EZH2:NM_152998.3:exon15:c.G1744A:p.V582M, | SNV | | |
| | | | EZH2:NM_001203247.2:exon16:c.G1861A:p.V621M, | | | |
| | | | EZH2:NM_001203248.2:exon16:c.G1834A:p.V612M, | | | |
| | | | EZH2:NM_004456.5:exon16:c.G1876A:p.V626M | | | |
| | | KRAS | KRAS:NM_001369786.1:exon2:c.G35A:p.G12D, | nonsynonymous | 18.78 | 0.0562249 |
| | | | KRAS:NM_001369787.1:exon2:c.G35A:p.G12D, | SNV | | |
| | | | KRAS:NM_004985.5:exon2:c.G35A:p.G12D, | | | |
| | | | KRAS:NM_033360.4:exon2:c.G35A:p.G12D | | | |
| | | NPM1 | NPM1:NM_001355010.1:exon5:c.374_386del:p.A126Kfs*16, | frameshift | 31.64 | 0.1607143 |
| | | | NPM1:NM_001355007.1:exon8:c.563_575del:p.A189Kfs*16, | deletion | | |
| | | | NPM1:NM_001355009.2:exon8:c.668_680del:p.A224Kfs*13, | | | |
| | | | NPM1:NM_199185.3:exon8:c.668_680del:p.A224Kfs*16, | | | |
| | | | NPM1:NM_001037738.3:exon9:c.755_767del:p.A253Kfs*13, | | | |
| | | | NPM1:NM_002520.7:exon9:c.755_767del:p.A253Kfs*16, | | | |
| | | | NPM1:NM_001355006.1:exon10:c.755_767del:p.A253Kfs*16 | | | |
| | | ZRSR2 | ZRSR2:NM_005089.4:exon8:c.C684G:p.S228R | nonsynonymous | 38.29 | 0.1597222 |
| | | | | SNV | | |
| D132 | BM | FBXW7 | FBXW7:NM_001013415.2:exon11:c.C1666T:p.R556W, | nonsynonymous | 19.77 | 0.1264368 |
| | | | FBXW7:NM_018315.5:exon11:c.C1780T:p.R594W, | SNV | | |
| | | | FBXW7:NM_033632.3:exon12:c.C2020T:p.R674W, | | | |
| | | | FBXW7:NM_001349798.2:exon14:c.C2020T:p.R674W | | | |
| | | IDH2 | IDH2:NM_001290114.2:exon2:c.G29A:p.R10Q, | nonsynonymous | 78.12 | 0.4337349 |
| | | | IDH2:NM_001289910.1:exon4:c.G263A:p.R88Q, | SNV | | |
| | | | IDH2:NM_002168.4:exon4:c.G419A:p.R140Q | | | |
| | | NPM1 | NPM1:NM_001355010.1:exon7:c.478_479insTCTG:p.W161Cfs*12, | frameshift | 45.26 | 0.4347826 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D133 | BM | TET2 | TET2:NM_001127208.3:exon6:c.C3646T:p.R1216X | stopgain | 113.17 | 0.4090909 |
| D134 | BM | GATA2 | GATA2:NM_001145662.1:exon5:c.G1072A:p.A358T, | nonsynonymous | 38.17 | 0.1842105 |
| | | | GATA2:NM_032638.5:exon5:c.G1114A:p.A372T, | SNV | | |
| | | | GATA2:NM_001145661.2:exon6:c.G1114A:p.A372T | | | |
| | | KRAS | KRAS:NM_001369786.1:exon2:c.G38A:p.G13D, | nonsynonymous | 29.57 | 0.173913 |
| | | | KRAS:NM_001369787.1:exon2:c.G38A:p.G13D, | SNV | | |
| | | | KRAS:NM_004985.5:exon2:c.G38A:p.G13D, | | | |
| | | | KRAS:NM_033360.4:exon2:c.G38A:pG13D | | | |
| | | PRPF40B | PRPF40B:NM_001379037.1:exon18:c.C1718T:p.S573L, | nonsynonymous | 106.37 | 0.5340909 |
| | | | PRPF40B:NM_001379035.1:exon19:c.C1883T:p.S628L, | SNV | | |
| | | | PRPF40B:NM_001379036.1:exon19:c.C1883T:p.S628L, | | | |
| | | | PRPF40B:NM_001379031.1:exon20:c.C1994T:p.S665L, | | | |
| | | | PRPF40B:NM_001379032.1:exon20:c.C1994T:p.S665L, | | | |
| | | | PRPF40B:NM_001379033.1:exon20:c.C1964T:p.S655L, | | | |
| | | | PRPF40B:NM_001379034.1:exon20:c.C1964T:p.S655L, | | | |
| | | | PRPF40B:NM_012272.3:exon20:c.C1970T:p.S657L, | | | |
| | | | PRPF40B:NM_001031698.3:exon21:c.C2075T:p.S692L, | | | |
| | | | PRPF40B:NM_001363607.2:exon21:c.C2075T:p.S692L, | | | |
| | | | PRPF40B:NM_001379030.1:exon21:c.C2045T:p.S682L | | | |
| D135 | BM | ASXL1 | ASXL1:NM_001363734.1:exon11:c.4432_4434del:p.V1479del, | nonframeshift | 197.87 | 0.9367089 |
| | | | ASXL1:NM_015338.6:exon12:c.4615_4617del:p.V1540del | deletion | | |
| | | CUX1 | CUX1:NM_001202544.3:exon10:c.851_855del:p.E284Gfs*38, | frameshift | 109.51 | 0.5517241 |
| | | | CUX1:NM_001202545.3:exon10:c.761_765del:p.E254Gfs*38, | deletion | | |
| | | | CUX1:NM_001202546.3:exon10:c.782_786del:p.E261Gfs*38, | | | |
| | | | CUX1:NM_001202543.2:exon11:c.899_903del:p.E300Gfs*38, | | | |
| | | | CUX1:NM_001913.5:exon11:c.899_903del:p.E300Gfs*38, | | | |
| | | | CUX1:NM_181500.4:exon11:c.893_897del:p.E298Gfs*38, | | | |
| | | | CUX1:NM_181552.4:exon11:c.866_870del:p.E289Gfs*38 | | | |
| | | EP300 | EP300:NM_001362843.2:exon2:c.C256T:p.R86X, | stopgain | 82.62 | 0.4868421 |
| | | | EP300:NM_001429.4:exon2:c.C256T:p.R86X | | | |
| | | FLT3 | FLT3:NM_004119.3:exon20:c.T2505G:p.D835E | nonsynonymous | 17.05 | 0.0841121 |
| | | | | SNV | | |
| | | NRAS | NRAS:NM_002524.5:exon2:c.G35T:p.G12V | nonsynonymous | 42.04 | 0.1640625 |
| | | | | SNV | | |
| | | SMC3 | SMC3:NM_005445.4:exon24:c.T2765C:p.L922P | nonsynonymous | 60.02 | 0.3888889 |
| | | | | SNV | | |
| | | SRSF2 | SRSF2:NM_001195427.2:exon1:c.C284A:p.P95H, | nonsynonymous | 76.45 | 0.4050633 |
| | | | SRSF2:NM_003016.4:exon1:c.C284A:p.P95H | SNV | | |
| D136 | BM | DNMT3A | DNMT3A:NM_001320893.1:exon14:c.1799_1801del:p.F600del, | nonframeshift | 200 | 0.489172 |
| | | | DNMT3A:NM_001375819.1:exon14:c.1586_1588del:p.F529del, | deletion | | |
| | | | DNMT3A:NM_153759.3:exon15:c.1688_1690del:p.F563del, | | | |
| | | | DNMT3A:NM_022552.5:exon19:c.2255_2257del:p.F752del, | | | |
| | | | DNMT3A:NM_175629.2:exon19:c.2255_2257del:p.F752del | | | |
| | | NPM1 | NPM1:NM_001355010.1:exon7:c.478_479insTCTG:p.W161Cfs*12, | frameshift | 117.27 | 0.4583333 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | TET2 | TET2:NM_001127208.3:exon11:c.C5681T:p.P1894L | nonsynonymous | 200 | 0.4383202 |
| | | | | SNV | | |
| D137 | BM | DNMT3A | DNMT3A:NM_001320893.1:exon4:c.C654G:p.Y218X, | stopgain | 64.86 | 0.3970588 |
| | | | DNMT3A:NM_001375819.1:exon4:c.C441G:p.Y147X, | | | |
| | | | DNMT3A:NM_153759.3:exon5:c.C543G:p.Y181X, | | | |
| | | | DNMT3A:NM_022552.5:exon9:c.C1110G:p.Y370X, | | | |
| | | | DNMT3A:NM_175629.2:exon9:c.C1110G:p.Y370X | | | |
| | | ETV6 | ETV6:NM_001987.5:exon7:c.T1166G:p.M389R | nonsynonymous | 42.49 | 0.3214286 |
| | | | | SNV | | |
| | | IDH1 | IDH1:NM_001282386.1:exon4:c.C394T:p.R132C, | nonsynonymous | 49.57 | 0.32 |
| | | | IDH1:NM_001282387.1:exon4:c.C394T:p.R132C, | SNV | | |
| | | | IDH1:NM_005896.4:exon4:c.C394T:p.R132C | | | |
| | | RUNX1 | RUNX1:NM_001001890.3:exon6:c.942dupC:p.I315Hfs*258, | frameshift | 15.95 | 0.1568627 |
| | | | RUNX1:NM_001754.5:exon9:c.1023dupC:p.I342Hfs*258 | insertion | | |
| D139 | BM | ATM | ATM:NM_000051.4:exon48:c.T7064A:p.V2355D, | nonsynonymous | 200 | 0.5183486 |
| | | | ATM:NM_001351834.2:exon49:c.T7064A:p.V2355D | SNV | | |
| | | FLT3 | FLT3:NM_004119.3:exon20:c.G2503C:p.D835H | nonsynonymous | 53.26 | 0.2696629 |
| | | | | SNV | | |
| | | PRF1 | PRF1:NM_001083116.3:exon2:c.G305T:p.C102F, | nonsynonymous | 98.81 | 0.5416667 |
| | | | PRF1:NM_005041.5:exon2:c.G305T:p.C102F | SNV | | |
| D141 | BM | EP300 | EP300:NM_001362843.2:exon20:c.3604dupA:p.E1203Rfs*9, | frameshift | 144.06 | 0.3944444 |
| | | | EP300:NM_001429.4:exon21:c.3682dupA:p.E1229Rfs*9 | insertion | | |
| | | TP53 | TP53:NM_001126115.1:exon1:c.G128A:p.R43H, | nonsynonymous | 200 | 0.797235 |
| | | | TP53:NM_001126116.1:exon1:c.G128A:pR43H, | SNV | | |
| | | | TP53:NM_001126117.1:exon1:c.G128A:p.R43H, | | | |
| | | | TP53:NM_001276697.2:exon1:c.G47A:p.R16H, | | | |
| | | | TP53:NM_001276698.2:exon1:c.G47A:p.R16H, | | | |
| | | | TP53:NM_001276699.2:exon1:c.G47A:p.R16H, | | | |
| | | | TP53:NM_001126118.1:exon4:c.G407A:p.R136H, | | | |
| | | | TP53:NM_000546.6:exon5:c.G524A:p.R175H, | | | |
| | | | TP53:NM_001126112.2:exon5:c.G524A:p.R175H, | | | |
| | | | TP53:NM_001126113.2:exon5:c.G524A:p.R175H, | | | |
| | | | TP53:NM_001126114.2:exon5:c.G524A:p.R175H, | | | |
| | | | TP53:NM_001276695.2:exon5:c.G407A:p.R136H, | | | |
| | | | TP53:NM_001276696.2:exon5:c.G407A:p.R136H, | | | |
| | | | TP53:NM_001276760.2:exon5:c.G407A:p.R136H, | | | |
| | | | TP53:NM_001276761.2:exon5:c.G407A:p.R136H | | | |
| D143 | BM | C17orf97 | C17orf97:NM_001013672.5:exon2:c.401_404del:p.Q136Afs*23 | frameshift | 141.58 | 0.3348624 |
| | | | | deletion | | |
| | | CEBPA | CEBPA:NM_001285829.1:exon1:c.592_593insAGC:p.E197_{—} | nonframeshift | 136.93 | 0.4482759 |
| | | | L198insQ, CEBPA:NM_001287424.2:exon1:c.1054_{—} | insertion | | |
| | | | 1055insAGC:p.E351L352insQ, CEBPA:NM_{—} | | | |
| | | | 001287435.1:exon1:c.907_908insAGC:p.E302_L303insQ, | | | |
| | | | CEBPA:NM_004364.5:exon1:c.949_950insAGC:p.E316_L317insQ | | | |
| | | CEBPA | CEBPA:NM_001287424.2:exon1:c.208_215del:p.R70Gfs*70, | frameshift | 169.48 | 0.4108911 |
| | | | CEBPA:NM_001287435.1:exon1:c.61_68del:p.R21Gfs*70, | deletion | | |
| | | | CEBPA:NM_004364.5:exon1:c.103_110del:p.R35Gfs*70 | | | |
| | | WT1 | WT1:NM_000378.6:exon1:c.486_493del:p.S163Hfs*38, | frameshift | 136.57 | 0.4203822 |
| | | | WT1:NM_024424.5:exon1:c.486_493del:p.S163Hfs*38, | deletion | | |
| | | | WT1:NM_024426.6:exon1:c.486_493del:p.S163Hfs*38 | | | |
| | | WT1 | WT1:NM_000378.6:exon1:c.638delG:p.S213Tfs*78, | frameshift | 200 | 0.4575472 |
| | | | WT1:NM_024424.5:exon1:c.638delG:p.S213Tfs*78, | deletion | | |
| | | | WT1:NM_024426.6:exon1:c.638delG:p.S213Tfs*78 | | | |
| D144 | BM | NPM1 | NPM1:NM_001355010.1:exon7:c.478_479insTCTG:p.W161Cfs*12, | frameshift | 22.46 | 0.3333333 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | TET2 | TET2:NM_001127208.3:exon5:c.3593delG:p.V1199Wfs*27 | frameshift | 90.07 | 0.4880952 |
| | | | | deletion | | |
| D145 | BM | ASXL1 | ASXL1:NM_001363734.1:exon11:c.2092_{—} | frameshift | 200 | 0.4814815 |
| | | | 2093insGCCA:p.A700Pfs*14, ASXL1:NM_{—} | insertion | | |
| | | | 015338.6:exon12:c.2275_2276insGCCA:p.A761Pfs*14 | | | |
| | | NPM1 | NPM1:NM_001355010.1:exon7:c.478_479insTCTG:p.W161Cfs*12, | frameshift | 51.7 | 0.3424658 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | SRSF2 | SRSF2:NM_001195427.2:exon1:c.C284T:p.P95L, | nonsynonymous | 164.8 | 0.4842767 |
| | | | SRSF2:NM_003016.4:exon1:c.C284T:p.P95L | SNV | | |
| | | TET2 | TET2:NM_001127208.3:exon3:c.2880_{—} | frameshift | 182.6 | 0.3888889 |
| | | | 2881insGAACAGCAGC:p.Q964Rfs*11, TET2:NM_{—} | | | |
| | | | 017628.4:exon3:c.2880_2881insGAACAGCAGC:p.Q964Rfs*11 | insertion | | |
| | | TET2 | TET2:NM_001127208.3:exon4:c.G3492A:p.M1164I | nonsynonymous | 200 | 0.5050167 |
| | | | | SNV | | |
| D146 | BM | KIT | KIT:NM_000222.3:exon17:c.A2447T:p.D816V, | nonsynonymous | 200 | 0.4264264 |
| | | | KIT:NM_001093772.2:exon17:c.A2435T:p.D812V, | SNV | | |
| | | | KIT:NM_001385284.1:exon17:c.A2450T:p.D817V, | | | |
| | | | KIT:NM_001385285.1:exon17:c.A2444T:p.D815V, | | | |
| | | | KIT:NM_001385286.1:exon17:c.A2432T:p.D811V, | | | |
| | | | KIT:NM_001385288.1:exon17:c.A2438T:p.D813V, | | | |
| | | | KIT:NM_001385290.1:exon17:c.A2447T:p.D816V, | | | |
| | | | KIT:NM_001385292.1:exon17:c.A2435T:p.D812V | | | |
| | | KMT2A | KMT2A:NM_001197104.2:exon27:c.G8432A:p.R2811H, | nonsynonymous | 200 | 0.4820847 |
| | | | KMT2A:NM_005933.4:exon27:c.G8423A:p.R2808H | SNV | | |
| | | KMT2C | KMT2C:NM_170606.3:exon17:c.G2770A:p.V924M | nonsynonymous | 200 | 0.5393258 |
| | | | | SNV | | |
| D147 | BM | ATM | ATM:NM_000051.4:exon50:c.C7311A:p.Y2437X, | stopgain | 98.76 | 0.4607843 |
| | | | ATM:NM_001351834.2:exon51:c.C7311A:p.Y2437X | | | |
| | | ATM | ATM:NM_000051.4:exon4:c.A274G:p.K92E, | nonsynonymous | 200 | 0.516129 |
| | | | ATM:NM_001351835.1:exon4:c.A274G:p.K92E, | SNV | | |
| | | | ATM:NM_001351836.1:exon4:c.A274G:p.K92E, | | | |
| | | | ATM:NM_001351834.2:exon5:c.A274G:p.K92E | | | |
| | | FLT3 | FLT3:NM_004119.3:exon14:c.1800_{—} | nonframeshift | 114.38 | 0.3916084 |
| | | | 1801insTTCAGAGAATATGAATATGAT:p.D600_{—} | insertion | | |
| | | | L601insFREYEYD | | | |
| | | IDH2 | IDH2:NM_001290114.2:exon2:c.G29A:p.R10Q, | nonsynonymous | 116.24 | 0.4435484 |
| | | | IDH2:NM_001289910.1:exon4:c.G263A:p.R88Q, | SNV | | |
| | | | IDH2:NM_002168.4:exon4:c.G419A:p.R140Q | | | |
| | | JAK2 | JAK2:NM_001322204.1:exon11:c.G1402T:p.V468F, | nonsynonymous | 103.66 | 0.5897436 |
| | | | JAK2:NM_001322195.1:exon13:c.G1849T:p.V617F, | SNV | | |
| | | | JAK2:NM_001322196.1:exon13:c.G1849T:p.V617F, | | | |
| | | | JAK2:NM_001322194.1:exon14:c.G1849T:p.V617F, | | | |
| | | | JAK2:NM_001322198.1:exon14:c.G634T:p.V212F, | | | |
| | | | JAK2:NM_001322199.1:exon14:c.G634T:p.V212F, | | | |
| | | | JAK2:NM_004972.4:exon14:c.G1849T:p.V617F | | | |
| | | NPM1 | NPM1:NM_001355010.1:exon7:c.478_479insTCTG:p.W161Cfs*12, | frameshift | 33.11 | 0.2909091 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| D148 | BM | BCORL1 | BCORL1:NM_001379450.1:exon4:c.2490delT:p.V831Lfs*22, | frameshift | 71.67 | 1 1 |
| | | | BCORL1:NM_001379451.1:exon4:c.2490delT:p.V831Lfs*22, | deletion | | |
| | | | BCORL1:NM_021946.5:exon4:c.2490delT:p.V831Lfs*22, | | | |
| | | | BCORL1:NM_001184772.3:exon5:c.2490delT:p.V831Lfs*22 | | | |
| | | FLT3 | FLT3:NM_004119.3:exon20:c.G2503T:p.D835Y | nonsynonymous | 41.74 | 0.4848485 |
| | | | | SNV | | |
| | | IDH1 | IDH1:NM_001282386.1:exon4:c.C394T:p.R132C, | nonsynonymous | 87.26 | 0.6545455 |
| | | | IDH1:NM_001282387.1:exon4:c.C394T:p.R132C, | SNV | | |
| | | | IDH1:NM_005896.4:exon4:c.C394T:p.R132C | | | |
| | | WT1 | WT1:NM_001367854.1:exon5:c.C199T:p.R67X, | stopgain | 92.48 | 0.4343434 |
| | | | WT1:NM_000378.6:exon8:c.C1336T:p.R446X, | | | |
| | | | WT1:NM_001198552.2:exon8:c.C685T:p.R229X, | | | |
| | | | WT1:NM_001198551.1:exon9:c.C736T:p.R246X, | | | |
| | | | WT1:NM_024424.5:exon9:c.C1387T:p.R463X, | | | |
| | | | WT1:NM_024426.6:exon9:c.C1387T:p.R463X | | | |
| D149 | BM | CEBPA | CEBPA:NM_001287424.2:exon1:c.306_307insCTAC:p.I103Lfs*41, | frameshift | 200 | 0.4859335 |
| | | | CEBPA:NM_001287435.1:exon1:c.159_160insCTAC:p.I54Lfs*41, | insertion | | |
| | | | CEBPA:NM_004364.5:exon1:c.201_202insCTAC:p.I68Lfs*41 | | | |
| | | GATA2 | GATA2:NM_001145662.1:exon4:c.C961T:p.L321F, | nonsynonymous | 200 | 0.4965278 |
| | | | GATA2:NM_032638.5:exon4:c.C961T:p.L321F, | SNV | | |
| | | | GATA2:NM_001145661.2:exon5:c.C961T:p.L321F | | | |
| | | SETBP1 | SETBP1:NM_001379141.1:exon6:c.A4187T:p.K1396M, | nonsynonymous | 200 | 0.5505051 |
| | | | SETBP1:NM_001379142.1:exon6:c.A4187T:p.K1396M, | SNV | | |
| | | | SETBP1:NM_015559.3:exon6:c.A4187T:p.K1396M | | | |
| D150 | BM | FLT3 | FLT3:NM_004119.3:exon14:c.T1775C:p.V592A | nonsynonymous | 22.76 | 0.0615942 |
| | | | | SNV | | |
| | | FLT3 | FLT3:NM_004119.3:exon14:c.T1775G:p.V592G | nonsynonymous | 22.76 | 0.2717391 |
| | | | | SNV | | |
| | | MSH6 | MSH6:NM_001281492.1:exon6:c.C3299G:p.A1100G, | nonsynonymous | 200 | 0.440678 |
| | | | MSH6:NM_001281493.1:exon7:c.C2783G:p.A928G, | SNV | | |
| | | | MSH6:NM_000179.3:exon8:c.C3689G:p.A1230G, | | | |
| | | | MSH6:NM_001281494.1:exon8:c.C2783G:p.A928G | | | |
| | | RUNX1 | RUNX1:NM_001001890.3:exon3:c.C520T:p.R174X, | stopgain | 170.46 | 0.5 |
| | | | RUNX1:NM_001122607.2:exon3:c.C520T:p.R174X, | | | |
| | | | RUNX1:NM_001754.5:exon6:c.C601T:p.R201X | | | |
| | | SF1 | SF1:NM_001346409.2:exon9:c.C755A:p.P252H, | nonsynonymous | 142.94 | 0.5114504 |
| | | | SF1:NM_001346410.2:exon9:c.C755A:p.P252H, | SNV | | |
| | | | SF1:NM_001178030.2:exon10:c.C1475A:p.P492H, | | | |
| | | | SF1:NM_001178031.3:exon10:c.C1022A:p.P341H, | | | |
| | | | SF1:NM_001346363.2:exon10:c.C1100A:p.P367H, | | | |
| | | | SF1:NM_001346364.2:exon10:c.C1100A:p.P367H, | | | |
| | | | SF1:NM_001378956.1:exon10:c.C1475A:p.P492H, | | | |
| | | | SF1:NM_001378957.1:exon10:c.C1475A:p.P492H, | | | |
| | | | SF1:NM_004630.4:exon10:c.C1100A:p.P367H, | | | |
| | | | SF1:NM_201995.3:exon10:c.C1100A:p.P367H, | | | |
| | | | SF1:NM_201997.3:exon10:c.C1100A:p.P367H, | | | |
| | | | SF1:NM_201998.3:exon10:c.C1100A:p.P367H | | | |
| | | SRP72 | SRP72:NM_001267722.2:exon8:c.G788T:p.R263L, | nonsynonymous | 177.59 | 0.5503356 |
| | | | SRP72:NM_006947.4:exon10:c.G971T:p.R324L | SNV | | |
| D151 | BM | JAK1 | JAK1:NM_001321852.2:exon6:c.A548G:p.H183R, | nonsynonymous | 200 | 0.4643963 |
| | | | JAK1:NM_001321856.1:exon6:c.A548G:p.H183R, | SNV | | |
| | | | JAK1:NM_001321857.2:exon6:c.A548G:p.H183R, | | | |
| | | | JAK1:NM_002227.4:exon6:c.A548G:p.H183R, | | | |
| | | | JAK1:NM_001320923.1:exon7:c.A548G:p.H183R, | | | |
| | | | JAK1:NM_001321854.2:exon7:c.A548G:p.H183R, | | | |
| | | | JAK1:NM_001321855.2:exon7:c.A548G:p.H183R, | | | |
| | | | JAK1:NM_001321853.2:exon8:c.A548G:p.H183R | | | |
| | | KIT | KIT:NM_000222.3:exon8:c.1253_1255del:p.D419del, | nonframeshift | 71.92 | 0.1256545 |
| | | | KIT:NM_001093772.2:exon8:c.1253_1255del:p.D419del, | deletion | | |
| | | | KIT:NM_001385284.1:exon8:c.1256_1258del:p.D420del, | | | |
| | | | KIT:NM_001385285.1:exon8:c.1253_1255del:p.D419del, | | | |
| | | | KIT:NM_001385286.1:exon8:c.1253_1255del:p.D419del, | | | |
| | | | KIT:NM_001385288.1:exon8:c.1256_1258del:p.D420del, | | | |
| | | | KIT:NM_001385290.1:exon8:c.1256_1258del:p.D420del, | | | |
| | | | KIT:NM_001385292.1:exon8:c.1256_1258del:p.D420del | | | |
| | | SMC3 | SMC3:NM_005445.4:exon16:c.T1645C:p.C549R | nonsynonymous | 96.73 | 0.1588542 |
| | | | | SNV | | |
| | | TET2 | TET2:NM_001127208.3:exon3:c.1915dupA:p.N639Kfs*42, | frameshift | 200 | 0.8450704 |
| | | | TET2:NM_017628.4:exon3:c.1915dupA:p.N639Kfs*42 | insertion | | |
| D152 | BM | IDH2 | IDH2:NM_001290114.2:exon2:c.G29A:p.R10Q, | nonsynonymous | 121.53 | 0.5698925 |
| | | | IDH2:NM_001289910.1:exon4:c.G263A:p.R88Q, | SNV | | |
| | | | IDH2:NM_002168.4:exon4:c.G419A:p.R140Q | | | |
| | | NPM1 | NPM1:NM_001355010.1:exon7:c.478_479insTCTG:p.W161Cfs*12, | frameshift | 27.42 | 0.3636364 |
| | | | NPM1:NM_001355007.1:exon10:c.667_668insTCTG:p.W224Cfs*12, | insertion | | |
| | | | NPM1:NM_199185.3:exon10:c.772_773insTCTG:p.W259Cfs*12, | | | |
| | | | NPM1:NM_002520.7:exon11:c.859_860insTCTG:p.W288Cfs*12, | | | |
| | | | NPM1:NM_001355006.1:exon12:c.859_860insTCTG:p.W288Cfs*12 | | | |
| | | PRPF40B | PRPF40B:NM_001379037.1:exon14:c.T1304G:p.M435R, | nonsynonymous | 107.31 | 0.5675676 |
| | | | PRPF40B:NM_001379035.1:exon15:c.T1469G:p.M490R, | SNV | | |
| | | | PRPF40B:NM_001379036.1:exon15:c.T1469G:p.M490R, | | | |
| | | | PRPF40B:NM_001379031.1:exon16:c.T1580G:p.M527R, | | | |
| | | | PRPF40B:NM_001379032.1:exon16:c.T1580G:p.M527R, | | | |
| | | | PRPF40B:NM_001379033.1:exon16:c.T1550G:p.M517R, | | | |
| | | | PRPF40B:NM_001379034.1:exon16:c.T1550G:p.M517R, | | | |
| | | | PRPF40B:NM_012272.3:exon16:c.T1577G:p.M526R, | | | |
| | | | PRPF40B:NM_001031698.3:exon17:c.T1661G:p.M554R, | | | |
| | | | PRPF40B:NM_001363607.2:exon17:c.T1661G:p.M554R, | | | |
| | | | PRPF40B:NM_001379030.1:exon17:c.T1631G:p.M544R | | | |
| D153 | BM | DNMT3A | DNMT3A:NM_001320893.1:exon9:c.G1171T:p.G391C, | nonsynonymous | 200 | 0.4961538 |
| | | | DNMT3A:NM_001375819.1:exon9:c.G958T:p.G320C, | SNV | | |
| | | | DNMT3A:NM_153759.3:exon10:c.G1060T:p.G354C, | | | |
| | | | DNMT3A:NM_022552.5:exon14:c.G1627T:p.G543C, | | | |
| | | | DNMT3A:NM_175629.2:exon14:c.G1627T:p.G543C | | | |
| | | IDH1 | IDH1:NM_001282386.1:exon4:c.C394T:p.R132C, | nonsynonymous | 43.12 | 0.4318182 |
| | | | IDH1:NM_001282387.1:exon4:c.C394T:p.R132C, | SNV | | |
| | | | IDH1:NM_005896.4:exon4:c.C394T:p.R132C | | | |
| | | PHF6 | PHF6:NM_001015877.2:exon10:c.1000_1003del:p.R335Mfs*15, | frameshift | 34.58 | 0.2142857 |
| | | | PHF6:NM_032458.3:exon10:c.1000_1003del:p.R335Mfs*15 | deletion | | |
| D154 | BM | DNMT3A | DNMT3A:NM_001320893.1:exon14:c.C1855T:p.R619X, | stopgain | 200 | 0.5078534 |
| | | | DNMT3A:NM_001375819.1:exon14:c.C1642T:p.R548X, | | | |
| | | | DNMT3A:NM_153759.3:exon15:c.C1744T:p.R582X, | | | |
| | | | DNMT3A:NM_022552.5:exon19:c.C2311T:p.R771X, | | | |
| | | | DNMT3A:NM_175629.2:exon19:c.C2311T:p.R771X | | | |
| | | DNMT3A | DNMT3A:NM_001320893.1:exon18:c.G2227A:p.V743M, | nonsynonymous | 200 | 0.4917695 |
| | | | DNMT3A:NM_001375819.1:exon18:c.G2014A:p.V672M, | SNV | | |
| | | | DNMT3A:NM_153759.3:exon19:c.G2116A:p.V706M, | | | |
| | | | DNMT3A:NM_022552.5:exon23:c.G2683A:p.V895M, | | | |
| | | | DNMT3A:NM_175629.2:exon23:c.G2683A:p.V895M | | | |
| | | IDH1 | IDH1:NM_001282386.1:exon4:c.C394T:p.R132C, | nonsynonymous | 146.9 | 0.4693878 |
| | | | IDH1:NM_001282387.1:exon4:c.C394T:p.R132C, | SNV | | |
| | | | IDH1:NM_005896.4:exon4:c.C394T:p.R132C | | | |
| D155 | BM | BRCA2 | BRCA2:NM_000059.4:exon19:c.G8356A:p.A2786T | nonsynonymous | 56.38 | 0.5333333 |
| | | | | SNV | | |
| | | NRAS | NRAS:NM_002524.5:exon2:c.G38A:p.G13D | nonsynonymous | 81.87 | 0.3539823 |
| | | | | SNV | | |
| D156 | BM | ASXL1 | NM_015338.6:exon12:c.1720 − 1G > C; | splice site | 48.69 | 0.3134328 |
| | | | NM_001363734.1:exon11:c.1537 − 1G > C | mutation | | |
| | | BCORL1 | BCORL1:NM_001379450.1:exon4:c.2079dupC:p.V694Rfs*48, | frameshift | 39.97 | 0.3166667 |
| | | | BCORL1:NM_001379451.1:exon4:c.2079dupC:p.V694Rfs*48, | insertion | | |
| | | | BCORL1:NM_021946.5:exon4:c.2079dupC:p.V694Rfs*48, | | | |
| | | | BCORL1:NM_001184772.3:exon5:c.2079dupC:p.V694Rfs*48 | | | |
| | | BRCA2 | BRCA2:NM_000059.4:exon25:c.T9299C:p.L3100S | nonsynonymous | 56.91 | 0.36 |
| | | | | SNV | | |
| | | EED | EED:NM_001308007.1:exon9:c.T956A:p.1319K, | nonsynonymous | 25.71 | 0.3703704 |
| | | | EED:NM_003797.5:exon9:c.T956A:p.I319K | SNV | | |
| | | EED | EED:NM_001308007.1:exon9:c.A906C:p.R302S, | nonsynonymous | 56.04 | 0.4035088 |
| | | | EED:NM_003797.5:exon9:c.A906C:p.R302S | SNV | | |
| | | NRAS | NRAS:NM_002524.5:exon2:c.G35A:p.G12D | nonsynonymous | 29.23 | 0.2083333 |
| | | | | SNV | | |
| | | RUNX1 | RUNX1:NM_001001890.3:exon2:c.317_318insCA:p.M106Ifs*13, | frameshift | 40.22 | 0.2258065 |
| | | | RUNX1:NM_001122607.2:exon2:c.317_318insCA:p.M106Ifs*13, | insertion | | |
| | | | RUNX1:NM_001754.5:exon5:c.398_399insCA:p.M133Ifs*13 | | | |
| | | RUNX1 | RUNX1:NM_001001890.3:exon5:c.C774G:p.Y258X, | stopgain | 71.77 | 0.3076923 |
| | | | RUNX1:NM_001754.5:exon8:c.C855G:p.Y285X | | | |
| D157 | BM | TET2 | TET2:NM_001127208.3:exon3:c.2962delA:p.K988Sfs*19, | frameshift | 122.06 | 0.3896104 |
| | | | TET2:NM_017628.4:exon3:c.2962delA:p.K988Sfs*19 | deletion | | |
| | | TP53 | TP53:NM_001126115.1:exon3:c.C326G:p.S109C, | nonsynonymous | 164.32 | 0.7727273 |
| | | | TP53:NM_001126116.1:exon3:c.C326G:p.S109C, | SNV | | |
| | | | TP53:NM_001126117.1:exon3:c.C326G:p.S109C, | | | |
| | | | TP53:NM_001276697.2:exon3:c.C245G:p.S82C, | | | |
| | | | TP53:NM_001276698.2:exon3:c.C245G:p.S82C, | | | |
| | | | TP53:NM_001276699.2:exon3:c.C245G:p.S82C, | | | |
| | | | TP53:NM_001126118.1:exon6:c.C605G:p.S202C, | | | |
| | | | TP53:NM_000546.6:exon7:c.C722G:p.S241C, | | | |
| | | | TP53:NM_001126112.2:exon7:c.C722G:p.S241C, | | | |
| | | | TP53:NM_001126113.2:exon7:c.C722G:p.S241C, | | | |
| | | | TP53:NM_001126114.2:exon7:c.C722G:p.S241C, | | | |
| | | | TP53:NM_001276695.2:exon7:c.C605G:p.S202C, | | | |
| | | | TP53:NM_001276696.2:exon7:c.C605G:p.S202C, | | | |
| | | | TP53:NM_001276760.2:exon7:c.C605G:p.S202C, | | | |
| | | | TP53:NM_001276761.2:exon7:c.C605G:p.S202C | | | |
| D158 | BM | CEBPA | CEBPA:NM_001285829.1:exon1:c.499delC:p.R167Gfs*32, | frameshift | 85.49 | 0.35 |
| | | | CEBPA:NM_001287424.2:exon1:c.961delC:p.R321Gfs*32, | deletion | | |
| | | | CEBPA:NM_001287435.1:exon1:c.814delC:p.R272Gfs*32, | | | |
| | | | CEBPA:NM_004364.5:exon1:c.856delC:p.R286Gfs*32 | | | |
| | | TET2 | TET2:NM_001127208.3:exon3:c.1837dupG:p.L615Afs*23, | frameshift | 57.29 | 0.2521008 |
| | | | TET2:NM_017628.4:exon3:c.1837dupG:p.L615Afs*23 | insertion | | |
| | | TET2 | TET2:NM_001127208.3:exon11:c.G5541A:p.W1847X | stopgain | 87.57 | 0.352459 |
| D159 | BM | KIT | KIT:NM_000222.3:exon9:c.C1463T:p.T488M, | nonsynonymous | 135.18 | 0.5350877 |
| | | | KIT:NM_001093772.2:exon9:c.C1463T:p.T488M, | SNV | | |
| | | | KIT:NM_001385284.1:exon9:c.C1466T:p.T489M, | | | |
| | | | KIT:NM_001385285.1:exon9:c.C1463T:p.T488M, | | | |
| | | | KIT:NM_001385286.1:exon9:c.C1463T:p.T488M, | | | |
| | | | KIT:NM_001385288.1:exon9:c.C1466T:p.T489M, | | | |
| | | | KIT:NM_001385290.1:exon9:c.C1466T:p.T489M, | | | |
| | | | KIT:NM_001385292.1:exon9:c.C1466T:p.T489M | | | |
| | | KMT2C | KMT2C:NM_170606.3:exon52:c.C13522A:p.P4508T | nonsynonymous | 200 | 0.9453552 |
| | | | | SNV | | |
| D160 | BM | DNMT3A | DNMT3A:NM_001320893.1:exon4:c.A643T:p.K215X, | stopgain | 178.8 | 0.462963 |
| | | | DNMT3A:NM_001375819.1:exon4:c.A430T:p.K144X, | | | |
| | | | DNMT3A:NM_153759.3:exon5:c.A532T:p.K178X, | | | |
| | | | DNMT3A:NM_022552.5:exon9:c.A1099T:p.K367X, | | | |
| | | | DNMT3A:NM_175629.2:exon9:c.A1099T:p.K367X | | | |
| | | FLT3 | FLT3:NM_004119.3:exon20:c.T2505G:p.D835E | nonsynonymous | 105.3 | 0.3982301 |
| | | | | SNV | | |
| | | RAD21 | RAD21:NM_006265.3:exon10:c.T1176A:p.C392X | stopgain | 99.13 | 0.407767 |

Abbreviations:
BM, bone marrow;
PB, peripheral blood;
SNV, single nucleotide variant;
VAF, variant allele frequency

1. A method for identifying high-risk Acute Myeloid Leukemia (AML) patients based upon a leukemic stem cell associated gene (LSCAG) known as Serine Protease Inhibitor Kazal type 2 (SPINK2), comprising of:
obtaining specimens from the AML patients;
performing immunohistochemistry (IHC) to detect SPINK2 expression; and,
quantifying the SPINK2 expression to identify the high-risk AML patients and low-risk AML patients by generating a range of IHC scores.

2. The method according to claim 1, wherein step of quantifying the SPINK2 expression to identify the high-risk AML patients and low-risk AML patients by generating the range of the IHC scores further comprising of:
generating the scores ranging from minimum 0 to maximum 16 based on level of the SPINK2 expression; and,
classifying the patients based on the level of the SPINK2 expression as “high-risk” if the patients score more than 3, or “low-risk” if the patients score less than or equal to 3.

3. The method according to claim 1, wherein the SPINK2 expression serves as a biomarker configured to determine level of risks of AML patients.

4. The method as claimed in claim 1, wherein the step of performing the immunohistochemistry (IHC) to detect SPINK2 expression further comprising of:
preparing stained slides with collected specimens and SPINK2 antibody including visualising using an IHC Detection Kit;
assessing the SPINK2-stained slides by employing percentage of stained blasts (P) with values of P: <20%=1, 20-50%=2,50-75%=3, >75%=4 and intensity of staining (I) with values of I: negative-0, mild-1, moderate-2, strong-3, very strong-4; and, calculating a unique IHC-score as “P×1” for each patient to obtain the IHC score.

5. A method for inhibiting proliferation of and inducing death in a leukemic cell comprising of contacting said leukemic cell with a small molecule inhibitor (SMI) wherein said leukemic cell expresses an elevated amount of SPINK2.

6. The method according to claim 5, wherein the SMI is screened via a structure-based virtual screening (SBVS) and selected from a group of bioactive molecules due to its efficient binding affinity based on its idock scores and its capacity to dissolve in at least one solvent selected from a group comprising of dimethyl sulfoxide (DMSO), water, ethanol or dimethylformamide (DMF).

7. A method for treating a patient with high-risk Acute Myeloid Leukemia (AML) identified as a potential candidate for receiving a small molecule inhibitor (SMI) therapy based upon its SPINK2 IHC score, the method comprising of:
administering to a patient an effective amount of the SMI;
wherein, the effective amount of the SMI selectively targets a domain of the SPINK2 in the leukemic cell which expresses SPINK2, and, the SMI reduces SPINK2 expression, consequently alters SPINK2 target gene mRNA expressions, thus inhibiting proliferation of and inducing death in the leukemic cell.

8. The method according to claim 7, wherein the altered SPINK2 target gene mRNA expressions are downregulation of SLC7A11 and upregulation of STEAP 3.

9. The method according to claim 7, wherein the SMI is administered to the patient as a single agent or in a combination with an existing treatment regimen including but not limited to erastin.

10. A pharmaceutical composition for treating Acute Myeloid Leukemia (AML) comprising of an effective amount of a small molecule inhibitor (SMI) or its pharmaceutically acceptable salt.

11. The pharmaceutical composition according to claim 10, wherein the composition is further comprising of an existing treatment regimen including but not limited to erastin.

12. A small molecule inhibitor (SMI) having a chemical structure of 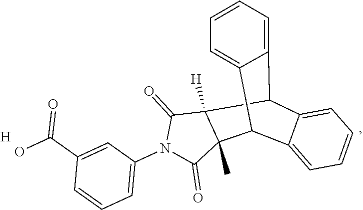
and molecular weight of 409.44 g/m and a chemical name of 3-[(15R,19S)-15-methyl-16,18-dioxo-17-azapentacyclo [6.6.5.02,7.09,14.015,19] nonadeca-2,4,6,9,11,13-hexaen-17-yl]benzoic acid for targeting SPINK2 and reducing its expression in a leukemic cell, or for both inhibiting proliferation and inducing death in the cell.
